(19) United States
(12) Patent Application Publication
(10) Pub. No.: US 2003/0035790 A1
(43) Pub. Date: Feb. 20, 2003

(54) Combination therapy for the prevention or treatment of cancer, inflammatory disorders or infectious diseases in a subject

(76) Inventors: Shu-Hsia Chen, New York, NY (US); Ping-Yan Pan, New York, NY (US); Savio L.C. Woo, New York, NY (US)

Correspondence Address: PENNIE AND EDMONDS, 1155 AVENUE OF THE AMERICAS, NEW YORK, NY, 100362711

(21) Appl. No.: 10/165,643

(22) Filed: Jun. 7, 2002

Related U.S. Application Data
(63) Continuation-in-part of application No. 09/735,296, filed on Jan. 14, 2000.
(60) Provisional application No. 60/115,992, filed on Jan. 15, 1999.

(51) Int. Cl. A61K 48/00; A61K 38/20; A61K 39/395

(52) U.S. Cl. 424/85.2; 424/143.1; 514/44

(57) ABSTRACT

The present invention relates to compositions comprising compounds which augment activated immune cells, such as T-cells, dendritic cells and natural killer (“NK”) cells, and methods for the treatment or prevention of diseases and disorders, including cancer, inflammatory disorders, and infectious diseases, in a subject comprising the administration of said compositions to said subject. In particular, the present invention relates to methods for the treatment or prevention of diseases and disorders, including cancer, inflammatory disorders, and infectious diseases, in a subject comprising administrating to said subject one or more compounds that activate one or more cytokine receptors and one or more compounds that activate one or more co-stimulatory molecules expressed by activated immune cells. The present invention also relates to compositions and kits comprising a compound that activates one or more cytokine receptors and a compound that activates one or more co-stimulatory molecules expressed by activated immune cells.

This application is a continuation-in-part of U.S. application Ser. No. 09/735,296, filed Jan. 14, 2000, which claims priority to U.S. provisional application Serial No. 60/115,992, filed Jan. 15, 1999, the entire contents of each of which is incorporated herein by reference.

### 1. FIELD OF THE INVENTION

The present invention relates to compositions comprising compounds which augment activated immune cells, such as T-cells, dendritic cells, and natural killer (“NK”) cells, and methods for the treatment or prevention of diseases and disorders, including cancer, inflammatory disorders, and infectious diseases, in a subject comprising the administration of said compositions to said subject. In particular, the present invention relates to methods for the treatment or prevention of diseases and disorders, including cancer, inflammatory disorders, and infectious diseases, in a subject comprising administrating to said subject one or more compounds that activate one or more cytokine receptors and one or more compounds that activate one or more co-stimulatory molecules expressed by activated immune cells. The present invention also relates to compositions and kits comprising a compound that activates one or more cytokine receptors and a compound that activates one or more co-stimulatory molecules expressed by activated immune cells.

### 2. BACKGROUND OF THE INVENTION

A neoplasm, or tumor, is a neoplastic mass resulting from abnormal uncontrolled cell growth, which can be benign or malignant. Benign tumors generally remain localized. Malignant tumors are collectively termed cancers. The term “malignant” generally means that the tumor can invade and destroy neighboring body structures and spread to distant sites and cause death (for review, see Robins and Angell, 1976, Basic Pathology, 2d Ed., W. B. Saunders Co., Philadelphia, pp. 68-122). A tumor is said to have metastatized when it has spread from one organ or tissue to another.

Cancer is the second leading cause of deaths in the United States. Carcinoma of the colon and rectum is second only to lung cancer as a major cause of cancer deaths. Prognosis for patients with metastatic disease in the liver and other organs is poor, and with current treatment, the mean survival time is only 3.7 years (Dreben, J. A. and Niederhuber, J. E., 1993, Colon Cancer In: Current Therapy in Oncology. Niederhuber, J. A. ex., B. C. Decker, St. Louis, 426-431; Lebovic, G. S., and Niederhuber, J. E., 1993, Colorectal cancer metastatic to the liver: Hepatic arterial infusion. In: Current Therapy in Oncology. Niederhuber, J.E., ed. B. C. Decker, St. Louis. 389-395; Fortner J. G., 1993, Colorectal cancer metastatic to the liver: Surgical Resection. In:, Current Therapy in Oncology. Niederhuber, J. E., ed. B. C. Decker, St. Louis; Kemeny, N. and Selter, K., 1993, Metastatic Colorectal Cancer: Chemotherapy. In: Current Therapy in Oncology. Niederhuber, J. E., ed. B. C. Decker, St. Louis. pp. 447-456). Therefore, a need exists for the development of alternative treatments for metastatic carcinoma than currently available.

One approach to the treatment of metastatic carcinoma is ex vivo gene therapy. In the ex vivo gene therapy or “cancer vaccine” approach, cancer cells are isolated from patients, transduced with various gene vectors and expanded in vitro. After irradiation, the cells are transplanted autologously to enhance the patient's immune response against the tumor. This strategy is not only laborious but the treatment is also individualized as cancer cells need to be cultured and expanded from each patient for therapeutic purposes. A more attractive strategy is to deliver the cytokine genes in vivo.

Cancer immunotherapy is a potent approach to combat metastatic diseases by stimulating a systemic anti-tumor response against disseminated tumor cells in the host. One reagent that has been shown to possess some anti-tumor activity when administered at the site of some murine tumors is B7-1 (Wu et al., 1995, J. Exp. Med. 182: 1415-1421; Chen et al., 1994, J Exp. Med. 179: 523-532). B7-1 is a ligand expressed on the surface of antigen-presenting cells (APCs) that binds to the CD28 receptor expressed on the surface of resting T-cells. The ability of B7-1 expression to induce an anti-tumor response is dependent on the type of tumor. Thus, B7-1 mediated immunotherapy is limited in its effectiveness in treatment of cancer.

One of the most promising reagents in cancer treatment to date is interleukin-12 (IL-12) due to its multiple regulatory effects. IL-12 is produced by antigen presenting cells (APC) such as macrophages, dendritic cells and B cells following appropriate stimulation. It plays an important role in orchestrating the host immune response by inducing interferon (IFN)-γ expression, promoting Thl cell differentiation, and enhancing T-cell, natural killer (NK) cell, lymphokine-activated killer (LAK), and macrophage mediated cytolytic activity (Banks et al., 1995, Br. J Cancer 71: 655-659; Brunda, M. J., 1994, Interleukin-12. J Leukocyte Biology 55: 280-288; Tsung et al., 1997, J. Immunology 158: 3359-3365; Scott, P., 1993, Science 260: 496-497; Nishmura et al., S., 1995, Immunology Letter 48: 167-174; Takeda et al., 1996, J. Immunology 156: 3366-3373; Cesano et al., 1993, J. Immunology 154: 2943-2957). In particular, IFN-γ induced IL-12 has been shown to enhance APC functions that are critical for IL-12 mediated therapy.

Caruso et al. demonstrated that intratumoral administration of a recombinant adenoviral vector expressing the murine IL-12 (Adv.mIL-12) results in high level expression of IL-12 at the tumor site and induces a strong anti-tumor immune response in a well established orthotopic murine colon carcinoma (MCA26) liver metastases model in syngeneic Balb/c mice (Caruso, M., Pham-Nguyen, K., Kwong, Y. L., Xu, B., Kosai, K. I., Finegold, M., Woo, S. L. C., and Chen, S. H., 1996, Proc. Natl. Acad. Sci. 93: 11302-11306). However, at the high doses of IL-12 gene expression needed to induce the long-term regression of established tumor, vector mediated IL-2 gene expression is toxic in animals (Putzer et al., 1997, Proc. Natl. Acad. Sci., USA 94: 10889-10894). Thus, vector mediated IL-12 gene application within a tumor is not effective in achieving tumor rejection.

Putzer et al. demonstrated that intratumoral administration of murine IL-12 and B7-1, a ligand for the co-stimulatory molecule CD28 which is expressed on resting T-cells, induces the regression of established tumors in a transgenic murine model of metastatic breast cancer and results in protective immunity against a second challenge with tumor cells (Putzer et al., 1997, Proc. Natl. Acad. Sci., USA 94: 10889-10894).

2.1 Co-Stimulatory Molecules

Co-stimulatory molecules such as 4-1BB, signaling lymphocyte activation molecule (SLAM), and OX-40 are expressed only or predominantly on activated T-cells. These co-stimulatory molecules have been suggested to act at different stages of T-cell activation or differentiation than CD28, or to promote the development of different effector functions than CD28 (Vinay et al., 1998, Seminars Immunology 10: 481-489; Aversa et al., 1997, J. Immunology 158: 4036-4044; Weinberg et al., 1998, Seminars Immunology 10: 471-480).

SLAM (or CDw150) is a member of the CD2 subfamily of the immunoglobulin superfamily and is expressed on the surface of activated T- and B-cells. SLAM upregulates IFN-γ and seems to act only on memory cells (Aversa et al., 1997, J. Immunology 158: 4036-4044).

OX-40 (or CD 134) expression is a member of the tumor necrosis factor receptor (TNFR) superfamily that binds to OX-40 ligand (OX-40L) expressed on antigen presenting cells, such as activated B-cells and dendritic cells. OX-40 expression is limited to activated CD4+T-cells. Co-stimulation of T-cells through OX-40 enhances T-cell proliferation and cytokine production. OX-40 has been suggested to play a role in sustaining proliferation of Th1 or Th2 effector cells and promoting the development of a Th2 response (Weinberg et al., 1998, Seminars Immunology 10: 471-480).

4-1BB glycoprotein is a member of the TNFR superfamily that binds to a high affinity ligand (4-1BB ligand) expressed on antigen presenting cells (APCs), such as dendritic cells, macrophages and activated B-cells (Vinay et al., 1998, Seminars Immunology 10: 481-489). 4-1BB is expressed on primed CD4+ and CD8+ T-cells (Goodwin, R. G., et al., 1993, Eur. J. Immunol. 23: 2631-2641; Pollok, K. E., et al., 1993, J. Immunol. 150: 771-781) after antigen or mitogen induction. Its interaction with 4-1BB ligand provides a strong signal for expansion of TCR ligated T-cells. It has been shown that systematic administration of an agonistic monoclonal antibody causes tumor reduction in s.c. tumor bearing animals, and both CD4+ and CD8+ T-cells are involved in the anti-tumor response (Melero et al., 1997, Nature Med. 3: 682-685; Melero et al., 1998, Eur. J. Immunol. 28: 1116-1121). However, anti-4-1BB antibody treatment is not adequate to sustain long term immunity.

Citation or identification of any reference in Section 2, or any section of this application shall not be construed as an admission that such reference is available as prior art to the present invention.

### 3. SUMMARY OF THE INVENTION

The present invention encompasses treatment protocols that provide a better therapeutic effect than currently existing clinical therapies for cancers, inflammatory disorders, and infectious diseases. The present invention provides combination therapies for the treatment or prevention of diseases and disorders, including cancer, inflammatory disorders, infectious diseases (e.g., microbial and viral infections) and diseases of the immune system, in a subject comprising the administration of compounds which augment activated immune cells (e.g., T-cells, dentritic cells, and natural killer (“NK”) cells) to said subject. In particular, the present invention provides combination therapies for the treatment or prevention of diseases and disorders, including cancer, inflammatory diseases or disorders, infectious diseases (e.g., microbial and viral infections) and diseases of the immune system, in a subject, wherein said combination therapies comprise administering to said subject one or more compounds that activate one or more cytokine receptors (i.e., one or more cytokine receptor-activating agents) and one or more compounds that activate one or more co-stimulatory molecules expressed by activated immune cells (i.e., one or more co-stimulatory molecule-activating agents). The present invention also provides combination therapies for the treatment or prevention of cancer, inflammatory disorders, and infectious diseases in a subject comprising administering to said subject one or more compounds that activate one or more cytokine receptors and one or more compounds that selectively activate activated T-cells (e.g., T-cells expressing ICOS, SLAM, CD25, CD30 and/or OX-40).

The combination therapies of the invention have an additive or synergistic therapeutic effect in a subject with cancer, an inflammatory disorder, or an infectious disease relative to the therapeutic effect of either a cytokine receptor-activating agent or a co-stimulatory molecule-activating agent alone. The combination therapies of the invention enable lower dosages and/or less frequent dosing of cytokine receptor-activating agents and/or co-stimulatory molecule-activating agents to be administered to a subject with cancer, an inflammatory disorder, or an infectious disease to achieve a therapeutic effect. The combination therapies of the invention reduce or avoid the adverse or unwanted side effects associated with the administration of cytokine receptor-activating agents and/or co-stimulatory molecule-activating agents.

The present invention provides methods for preventing or treating cancer, an inflammatory disorder, or an infectious disease in a subject, said methods comprising administering to a subject in need thereof an effective amount of one or more cytokine receptor-activating agents and an effective amount of one or more co-stimulatory molecule-activating agents. One or more cytokine receptor-activating agents may be administered to a subject with cancer, an inflammatory disorder or an infectious disease prior to (e.g., 2 minutes, 5 minutes, 10 minutes, 15 minutes, 30 minutes, 45 minutes, 60 minutes, 2 hours, 4 hours, 6 hours, 8 hours, 10 hours, 12 hours, 14 hours, 16 hours, 18 hours, 20 hours, 22 hours, 24 hours, 2 days, 4 days, 5 days, 7 days, 2 weeks, 4 weeks or 6 weeks before), concomitantly with, or subsequent to (e.g., 2 minutes, 5 minutes, 10 minutes, 15 minutes, 30 minutes, 45 minutes, 60 minutes, 2 hours, 4 hours, 6 hours, 8 hours, 10 hours, 12 hours, 14 hours, 16 hours, 18 hours, 20 hours, 22 hours, 24 hours, 2 days, 4 days, 5 days, 7 days, 2 weeks, 4 weeks or 6 weeks after) the administration of one or more co-stimulatory molecule-activating agents. Examples of cytokine receptor-activating agents include, but are not limited to, cytokines, nucleic acid molecules comprising nucleotide sequences encoding cytokines, agonistic antibodies that immunospecifically bind to a cytokine receptor, and nucleic acid molecules comprising nucleotide sequences encoding agonistic antibodies that immunospecifically bind to one or more subunits of a cytokine receptor. Examples of co-stimulatory molecule-activating agents include, but are not limited to, ligands for co-stimulatory molecules expressed by immune cells (preferably, activated immune cells such as activated T-cells), nucleic acid molecules comprising nucleotide sequences encoding ligands for co-stimulatory molecules expressed by immune cells (preferably, activated immune cells such as activated T-cells), agonistic antibodies that immunospecifically bind to a co-stimulatory molecule, nucleic acid molecules comprising nucleotide sequences encoding agonistic antibodies that immunospecifically bind to a co-stimulatory molecule.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or natural killer cells (NK) cells and an effective amount of one or more co-stimulatory molecule-activating agents. Preferably, the cytokine receptor-activating agent shifts the Th1/Th2 balance in a subject, and more preferably, the cytokine receptor-activating agent shifts the Th1/Th2 balance and induces the proliferation and/or differentiation of Th1 cells in a subject. In one embodiment, the present invention provides a method for preventing or treating cancer or an infectious disease in a subject comprising administering to said subject an effective amount one or more compounds that activate the IL-15 receptor and an effective amount of one or more co-stimulatory molecule-activating agents. In another embodiment, the present invention provides a method for preventing or treating cancer or an infectious disease in a subject comprising administering to said subject an effective amount one or more compounds that activate the IL-18 receptor and an effective amount of one or more co-stimulatory molecule-activating agents. In yet another embodiment, the present invention provides a method for preventing or treating cancer or an infectious disease in a subject comprising administering to said subject an effective amount one or more compounds that activate Flt3 and an effective amount of one or more co-stimulatory molecule-activating agents.

The present invention provides methods for preventing or treating cancer or an infectious disease in a subject, said methods comprising administering to a subject in need thereof an effective amount of a compound that activates the IL-12 receptor (e.g., IL-12 or anti-IL-12R antibodies) and an effective amount of a co-stimulatory molecule-activating agent. In one embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies) and an effective amount of one or more compounds that activate 4-1BB (e.g., 4-1BB ligand or anti-4-1BB antibody). In another embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies) and an effective amount of one or more compounds that activate OX40 (e.g., OX40 ligand or anti-OX40 antibody).

In a preferred embodiment, the present invention provides a method for preventing or treating cancer or an infectious disease in a subject, said method comprising administering to said subject an effective amount of a recombinant adenovirus engineered to express IL-12 and an effective amount of an agonistic anti-4-1BB monoclonal antibody or antigen-binding fragment thereof. In another preferred embodiment, the present invention provides a method for preventing or treating cancer or an infectious disease in a subject, said method comprising administering to said subject an effective amount of a recombinant adenovirus engineered to express IL-12 and an effective amount of an agonistic anti-OX40 monoclonal antibody or antigen-binding fragment thereof.

The present invention provides methods for preventing or treating cancer or an infectious disease in a subject, said methods comprising administering to a subject in need thereof an effective amount of one or more compounds that activate the IL-12 receptor (e.g., IL-12 or anti-IL-I 2R antibodies) and an effective amount of two or more co-stimulatory molecule-activating agents. In a preferred embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies), an effective amount of one or more compounds that activate 4-1BB (e.g., 4-1BB ligand or anti-4-1BB antibody), and an effective amount of one or more compounds that activate OX40 (e.g, OX40 ligand or anti-OX40 antibody). In another embodiment, the present invention provides a method for preventing or treating cancer or an infectious disease in a subject, said method comprising administering to said subject an effective amount of one or more compounds that activate the IL-12 receptor, an effective amount of one or more compounds that activate 4-1BB, and an effective amount of one or more compounds that activate SLAM, ICOS, B7RP-1 or CD27. In another embodiment, the present invention provides methods for preventing or treating cancer or an infectious disease in a subject, said method comprising administering to said subject an effective amount of one or more compounds that activate the IL-12 receptor, an effective amount of one or more compounds that activate OX40, and an effective amount of one or more compounds that activate SLAM, ICOS, B7RP-1 or CD27. In yet another embodiment, the present invention provides a method for preventing or treating cancer or an infectious disease in a subject, said method comprising administering to said subject an effective amount of one or more compounds that activate the IL-12 receptor, an effective amount of one or more compounds that activates 4-1BB, an effective amount of one or more compounds that activate OX40, and an effective amount of one or more compounds that activate SLAM, ICOS, B7RP-1 or CD27.

In a preferred embodiment, the present invention provides a method for preventing or treating cancer or an infectious disease in a subject, said method comprising administering to said subject an effective amount of a recombinant adenovirus engineered to express IL-12, an effective amount of an agonistic anti-4-1BB monoclonal antibody or antigen-binding fragment thereof, and an effective amount of an agonistic anti-OX40 monoclonal antibody or antigen-binding fragment thereof.

The present invention provides methods for preventing or treating cancer or an infectious disease in a subject, said methods comprising administering to a subject in need thereof an effective amount of one or more compounds that activate the IL-12 receptor, an effective amount of one or more compounds that activate at least one cytokine receptor other than the IL-12 receptor, and an effective amount of one or more co-stimulatory molecule-activating agents. In one embodiment, the present invention provides a method for preventing or treating cancer or an infectious disease in a subject, said method comprising administering to said subject an effective amount of one or more compounds that activate the IL-12 receptor, an effective amount of one or more compounds that activate at least one cytokine receptor other the IL-12 receptor (e.g., one or more cytokines such as IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, GM-CSF, G-CSF, CSF-1, and M-CSF), and an effective amount of one or more co-stimulatory molecule-activating agents. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-15 receptor, and an effective amount of one or more co-stimulatory molecule-activating agents. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-18 receptor, and an effective amount of one or more co-stimulatory molecule-activating agents. In yet another embodiment, the present invention provides a method for preventing or treating cancer or an infectious disease in a subject, said method comprising administering to said subject an effective amount of one or more compounds that activate the IL-12 receptor, an effective amount of one or more compounds that activate the Flt3, and an effective amount of one or more co-stimulatory molecule-activating agents.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, and an effective amount of one or more co-stimulatory molecule-activating agents which affect the biological activity (e.g., differentiation, proliferation or effector function) of dendritic cells and/or macrophages. In a specific embodiment, the present invention provides a method for preventing or treating cancer, an inflammatory disorder, or an infectious disease in a subject, said method comprising administering to a subject in need thereof an effective amount of one or more compounds that activate the GM-CSF receptor and an effective amount of one or more compounds that activate CD40. In another embodiment, the present invention provides a method for preventing or treating cancer, an inflammatory disorder, or an infectious disease in a subject, said method comprising administering to a subject in need thereof an effective amount of one or more compounds that activate the GM-CSF receptor and an effective amount of one or more compounds that activate 4-1BB.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, an effective amount of one or more cytokine receptor-activating agents which promote the differentiation of myeloid cells into dendritic cells and/or macrophages, and an effective amount of one or more co-stimulatory molecule-activating agents which affect the biological activity (e.g., differentiation, proliferation or effector function) of dendritic cells and/or macrophages. In one embodiment, the present invention provides a method for preventing or treating cancer, an inflammatory disorder, or an infectious disease in a subject, said method comprising administering to a subject in need thereof an effective amount of one or more compounds that activate the IL-12 receptor, an effective amount of one or more compounds that activate the GM-CSF receptor, and an effective amount of one or more compounds that activate CD40.

The present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of myeloid cells into dendritic cells and/or macrophages. Preferably, the cytokine receptor-activating agent which affects the biological activity of Th cells shifts the Th1/Th2 balance in a subject, and more preferably, the cytokine receptor-activating agent which affects the biological activity of Th cells shifts the Th1/Th2 balance and induces the proliferation and/or differentiation of Th1 cells in a subject.

In a preferred embodiment, the present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of Gr-1⁺ myeloid progenitor cells into dendritic cells and/or macrophages. In another preferred embodiment, the present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of Gr-1⁺/CD11b⁺ myeloid progenitor cells into dendritic cells and/or macrophages.

In a specific embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-3 receptor, IL-4 receptor, IL-6 receptor, Flt-3, GM-CSF receptor, M-CSF receptor G-CSF receptor, or CSF receptor, and an effective amount of one or more co-stimulatory molecule-activating agents. In a another embodiment, the present invention provides a method for preventing or treating cancer or an infectious disease in a subject, said method comprising administering to said subject an effective amount of one or more compounds that activate the IL-12 receptor, an effective amount of one or more compounds that activate the GM-CSF receptor, and an effective amount of one or more compounds that activate 4-1BB. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the GM-CSF receptor, and an effective amount of one or more compounds that activate OX40. In yet another embodiment, the present invention provides a method for preventing or treating cancer or an infectious disease in a subject, said method comprising administering to said subject an effective amount of one or more compounds that activate the IL-12 receptor, an effective amount of one or more compounds the activate the GM-CSF receptor, an effective amount of one or more compounds that activate 4-1BB, and an effective amount of one or more compounds that activate OX-40.

In another embodiment, the present invention provides a method for preventing or treating cancer or an infectious disease in a subject, said method comprising administering to said subject an effective amount of one or more compounds that activate the IL-12 receptor, an effective amount of one or more compounds the activate the Flt3, and an effective amount of one or more compounds that activate 4-1BB. In another embodiment, the present invention provides a method for preventing or treating cancer or an infectious disease in a subject, said method comprising administering to said subject an effective amount of one or more compounds that activate the IL-12 receptor, an effective amount of one or more compounds the activate the Flt3, and an effective amount of one or more compounds that activate OX40. In yet another embodiment, the present invention provides a method for preventing or treating cancer or an infectious disease in a subject, said method comprising administering to said subject an effective amount of one or more compounds that activate the IL-12 receptor, an effective amount of one or more compounds the activate the Flt3, an effective amount of one or more compounds that activate 4-1BB, and an effective amount of one or more compounds that activate OX40.

In a preferred embodiment, the present invention provides a method for preventing or treating cancer or an infectious disease in a subject, said method comprising administering to said subject an effective amount of a recombinant adenovirus engineered to express IL-12, an effective amount of a recombinant adenovirus engineered to express GM-CSF, and an effective amount of an agonistic anti-4-1BB monoclonal antibody or antigen-binding fragment thereof. In another preferred embodiment, the present invention provides a method for preventing or treating cancer or an infectious disease in a subject, said method comprising administering to said subject an effective amount of a recombinant adenovirus engineered to express IL-12, an effective amount of a recombinant adenovirus engineered to express GM-CSF, and an effective amount of an agonistic anti-OX40 monoclonal antibody or antigen-binding fragment thereof. In yet another preferred embodiment, the present invention provides a method for preventing or treating cancer or an infectious disease in a subject, said method comprising administering to said subject an effective amount of a recombinant adenovirus engineered to express IL-12, an effective amount of a recombinant adenovirus engineered to express GM-CSF, an effective amount of an agonistic anti-4-1BB monoclonal antibody or an antigen-binding fragment thereof, and an effective amount of an agonistic anti-OX40 monoclonal antibody or an antigen-binding fragment thereof.

The present invention provides methods for preventing or treating cancer, an inflammatory disorder, or an infectious disease in a subject, said methods comprising administering to a subject in need thereof an effective amount of one or more cytokine receptor-activating agents and an effective amount of at least one fusion protein, wherein the fusion protein comprises a co-stimulatory molecule-activating polypeptide fused a heterologous protein, polypeptide or peptide. The present invention also provides methods for preventing or treating cancer, an inflammatory disorder, or an infectious disease in a subject, said methods comprising administering to a subject in need thereof an effective amount of one or more co-stimulatory molecule-activating agents and an effective amount of at least one fusion protein, wherein the fusion protein comprises a cytokine receptor-activating polypeptide fused a heterologous protein, polypeptide or peptide. Nucleic acid molecules encoding fusion proteins may be administered to a subject with cancer, an inflammatory disorder or an infectious disease rather than the fusion proteins themselves.

The present invention also provides methods for preventing or treating cancer, an inflammatory disorder, or an infectious disease in a subject, said methods comprising administering to a subject in need thereof an effective amount of at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. In a specific embodiment, the present invention provides a method for preventing or treating cancer or an infectious disease in a subject, said method comprising administering to said subject an effective amount of at least two fusion proteins, wherein one of the fusion proteins comprises a cytokine receptor-activating polypeptide that activates the IL-12 receptor fused a heterologous protein, polypeptide or peptide, and the other fusion protein comprises a co-stimulatory molecule-activating polypeptide that activates 4-1BB or OX40 fused a heterologous protein, polypeptide or peptide.

The present invention provides methods for preventing or treating cancer in a subject, said methods comprising administering to a subject in need thereof an effective amount of one or more cytokine receptor-activating agents, an effective amount of one or more co-stimulatory molecule-activating agents, and at least one other known cancer therapy. In a specific embodiment, the present invention provides a method for preventing or treating cancer in a subject, said method comprising administering to said subject an effective amount of one or more cytokine receptor-activating agents, an effective amount of one or more co-stimulatory molecule-activating agents, and an effective amount of at least one other anti-cancer agent such as a chemotherapeutic agent or an antibody that immunospecifically binds to a cancer cell antigen. Examples of chemotherapeutic agents include, but are not limited to, cisplatin, ifosfamide, paclitaxol, taxanes, topoisomerase I inhibitors (e.g., CPT-11, topotecan, 9-AC, and GG-211), gemeitabine, vinorelbine, oxaliplatin, 5-fluorouracil (5-FU), leucovorin, vinorelbine, temodal, taxol, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, melphalan, glucocorticoids, procaine, tetracaine, lidocaine, propranolol, puromycin homologs, and cytoxan. Examples of antibodies which can be used in the treatment of cancer include, but are not limited to, Herceptin® (Trastuzumab; Genetech, Calif.) which is a humanized anti-HER2 monoclonal antibody for the treatment of patients with metastatic breast cancer; Retuxan® (rituximab; Genentech) which is a chimeric anti-CD20 monoclonal antibody for the treatment of patients with non-Hodgkin's lymphoma; OvaRex (AltaRex Corporation, MA) which is a murine antibody for the treatment of ovarian cancer; Panorex (Glaxo Wellcome, N.C.) which is a murine IgG2a antibody for the treatment of colorectal cancer; BEC2 (ImClone Systems Inc., NY) which is murine IgG antibody for the treatment of lung cancer; IMC-C225 (Imclone Systems Inc., NY) which is a chimeric IgG antibody for the treatment of head and neck cancer; Vitaxin (MedImmune, Inc., MD) which is a humanized antibody for the treatment of sarcoma; Campath I/H (Leukosite, Mass.) which is a humanized IgG₁ antibody for the treatment of chronic lymphocytic leukemia (CLL); Smart MI95 (Protein Design Labs, Inc., CA) which is a humanized IgG antibody for the treatment of acute myeloid leukemia (AML); LymphoCide (Immunomedics, Inc., NJ) which is a humanized IgG antibody for the treatment of non-Hodgkin's lymphoma; Smart I D 10 (Protein Design Labs, Inc., CA) which is a humanized antibody for the treatment of non-Hodgkin's lymphoma; and Oncolym (Techniclone, Inc., CA) which is a murine antibody for the treatment of non-Hodgkin's lymphoma.

The present invention provides methods for preventing or treating an inflammatory disorder in a subject, said methods comprising administering to a subject in need thereof an effective amount of one or more cytokine receptor-activating agents, an effective amount of one or more co-stimulatory molecule-activating agents, and at least one other known anti-inflammatory agent. Examples of anti-inflammatory agents include, but are not limited to, aspirin, non-steroidal anti-inflammatory agents (e.g., ibuprofen, fenoprofen, indomethacin, and naproxen), Cox-2 inhibitors (e.g., rofecoxib (Vioxx) and celecoxib (Celebrex)), and anti-TNFα agents (e.g., infliximab (Remicade) and etanercept (Enbrel)).

The present invention provides methods for preventing or treating an infectious disease in a subject, said methods comprising administering to a subject in need thereof an effective amount of one or more cytokine receptor-activating agents, an effective amount of one or more co-stimulatory molecule-activating agents, and at least one known anti-viral, anti-microbial agent or anti-fungal agent. Examples of antibodies used as anti-viral or anti-microbial agents for the treatment of viral infection or microbial infection include, but are not limited to, PRO542 (Progenics) which is a CD4 fusion antibody for the treatment of HIV infection; Ostavir (Protein Design Labs, Inc., CA) which is a human antibody for the treatment of hepatitis B virus; Protovir (Protein Design Labs, Inc., CA) which is a humanized IgG₁ antibody for the treatment of cytomegalovirus (CMV); and anti-LPS antibodies. Examples of antibiotics used as anti-microbial agents for the treatment of microbial infections include, but are not limited to, penicillin, amoxicillin, ampicillin, carbenicillin, ticarcillin, piperacillin, cepalospolin, vancomycin, tetracycline, erythromycin, amphotericin B, nystatin, metronidazole, ketoconazole, and pentamidine. Examples of drugs used for the treatment of viral infections include, but are not limited to, inhibitors of reverse transcriptase (e.g., AZT, 3TC, D4T, ddC, ddI, d4T, 3TC, adefovir, efavirenz, delavirdine, nevirapine, abacavir, and other dideoxynucleosides or dideoxyfluoronucleosides); inhibitors, of viral mRNA capping, such as ribavirin; inhibitors of proteases such HIV protease inhibitors (e.g., amprenavir, indinavir, nelfinavir, ritonavir, and saquinavir,); amphotericin B; castanospermine as an inhibitor of glycoprotein processing; inhibitors of neuraminidase such as influenza virus neuraminidase inhibitors (e.g., zanamivir and oseltamivir); topoisomerase I inhibitors (e.g., camptothecins and analogs thereof); amantadine; and rimantadine.

The invention provides therapeutic and pharmaceutical compositions comprising pharmaceutically acceptable carriers, one or more cytokine receptor-activating agents, and one or more co-stimulatory molecule-activating agents. The pharmaceutical compositions of the invention may be used in accordance with the methods of the invention for the treatment of cancer, an inflammatory disorder, or an infectious disease in a subject. Cytokine receptor-activating polypeptides can be supplied by direct administration or indirectly as “pro-drugs” using somatic cell gene therapy. Co-stimulatory molecule-activating polypeptides can also be supplied by direct administration or indirectly as “pro-drugs” using somatic cell gene therapy. The pharmaceutical compositions of the present invention are in suitable formulation to be administered to animals, preferably mammals such as companion animals (e.g., dogs, cats, and horses) and livestock (e.g., cows and pigs), and most preferably humans.

The present invention provides therapeutic or pharmaceutical compositions comprising a pharmaceutical carrier, one or more cytokine receptor-activating agents which affect the biological activity (e.g., differentiation, proliferation or effector function) of T helper (Th) cells and/NK cells, and one or more co-stimulatory molecule-activating agents. In a specific embodiment, a pharmaceutical composition comprises a pharmaceutical carrier, one or more compounds that activate the IL-15 receptor, and one or more co-stimulatory molecule-activating agents. In another embodiment, a pharmaceutical composition comprises a pharmaceutical carrier, one or more compounds that activate the IL-18 receptor, and one or more co-stimulatory molecule-activating agents. In yet another embodiment, a pharmaceutical composition comprises a pharmaceutical carrier, one or more compounds that activate Flt3, and one or more co-stimulatory molecule-activating agents.

The invention provides therapeutic and pharmaceutical compositions comprising pharmaceutically acceptable carriers, one or more compounds that activate the IL-12 receptor, and one or more co-stimulatory molecule-activating agents. In one embodiment, a pharmaceutical composition comprises a pharmaceutically acceptable carrier, one or more compounds that activate the IL-12 receptor, and one or more compounds that activate 4-1BB. In another embodiment, a pharmaceutical composition comprises a pharmaceutical carrier, a recombinant adenovirus expressing IL-12, and an agonistic anti-4-1BB antibody or an antigen-binding fragment thereof. In another embodiment, a pharmaceutical composition comprises a pharmaceutically acceptable carrier, one or more compounds that activate the IL-12 receptor, and an effective amount of one or more compounds that activate OX40. In another embodiment, a pharmaceutical composition comprises a pharmaceutical carrier, a recombinant adenovirus expressing IL-12, and an agonistic anti-OX40 monoclonal antibody or antigen-binding fragment thereof. In a preferred embodiment, a pharmaceutical composition comprises a pharmaceutically acceptable carrier, one or more compounds that activate the IL-12 receptor, one or more compounds that activate 4-1BB, and one or more compounds that activate OX40. In another preferred embodiment, a pharmaceutical composition comprises a pharmaceutical carrier, a recombinant adenovirus expressing IL-12, an agonistic anti-4-1BB monoclonal antibody or antigen-binding fragment thereof, and an agonistic anti-OX40 monoclonal antibody or antigen-binding fragment thereof.

In another embodiment, a pharmaceutical composition comprises a pharmaceutically acceptable carrier, one or more compounds that activate the IL-12 receptor, one or more compounds that activate 4-1BB, and one or more compounds that activate SLAM, ICOS, B7RP-1 or CD27. In another embodiment, a pharmaceutical composition comprises a pharmaceutically acceptable carrier, one or more compounds that activate the IL-12 receptor, one or more compounds that activate OX40, and one or more compounds that activate SLAM, ICOS, B7RP-1 or CD27. In yet another embodiment, a pharmaceutical composition comprises a pharmaceutically acceptable carrier, one or more compounds that activate the IL-12 receptor, one or more compounds that activate 4-1BB, one or more compounds that activate OX40, and one or more compounds that activate SLAM, ICOS, B7RP-1 or CD27.

The invention provides therapeutic and pharmaceutical compositions comprising pharmaceutically acceptable carriers, one or more compounds that activate the IL-12 receptor, one or more compounds that activate at least one cytokine receptor other than the IL-12 receptor, and one or more co-stimulatory molecule-activating agents. In one embodiment, a pharmaceutical composition comprising a pharmaceutical carrier, one or more compounds that activate the IL-12 receptor, one or more compounds that activate at least one cytokine receptor other the IL-12 receptor (e.g., one or more cytokines such as IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, GM-CSF, G-CSF, CSF-1, and M-CSF), and one or more co-stimulatory molecule-activating agents. In another embodiment, a pharmaceutical composition comprises a pharmaceutical carrier, one or more compounds that activate the IL-12 receptor, one or more compounds that activate the IL-15 receptor, and one or more co-stimulatory molecule-activating agents. In another embodiment, a pharmaceutical composition comprises a pharmaceutical carrier, one or more compounds that activate the IL-12 receptor, one or more compounds that activate the IL-18 receptor, and one or more co-stimulatory molecule-activating agents. In yet another embodiment, a pharmaceutical composition comprises a pharmaceutical carrier, one or more compounds that activate the IL-12 receptor, one or more compounds that activate Flt3, and one or more co-stimulatory molecule-activating agents.

The present invention provides therapeutic and pharmaceutical compositions comprising pharmaceutically acceptable carriers, one or more cytokine receptor-activating agents which affect the biological activity (e.g., differentiation, proliferation or effector function) of T helper (Th) cells and/or NK cells, and one or more co-stimulatory molecule-activating agents which affect the biological activity (e.g., differentiation, proliferation or effector function) of dendritic cells and/or macrophages. In a specific embodiment, the present invention provides a pharmaceutical composition comprising a pharmaceutically acceptable carrier, one or more compounds that activate the GM-CSF receptor and one or more compounds that activate CD40. In another embodiment, the present invention provides a pharmaceutical composition comprising a pharmaceutically acceptable carrier, one or more compounds that activate the GM-CSF receptor, and one or more compounds that activate 4-1BB.

The present invention provides therapeutic and pharmaceutical compositions comprising pharmaceutically acceptable carriers, one or more cytokine receptor-activating agents which affect the biological activity (e.g., differentiation, proliferation or effector function) of T helper (Th) cells and/or NK cells, an effective amount of one or more cytokine receptor-activating agents which promote the differentiation of myeloid cells into dendritic cells and/or macrophages, and an effective amount of one or more co-stimulatory molecule-activating agents which affect the biological activity (e.g., differentiation, proliferation or effector function) of dendritic cells and/or macrophages. In one embodiment, the present invention provides a pharmaceutical composition comprising a pharmaceutically acceptable carrier, one or more compounds that activate the IL-12 receptor, an effective amount of one or more compounds that activate the GM-CSF receptor, and one or more compounds that activate CD40.

The present invention provides therapeutic or pharmaceutical compositions comprising a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of myeloid cells into dendritic cells and/or macrophages. In a preferred embodiment, a pharmaceutical composition comprises a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of Gr-1⁺ myeloid progenitor cells into dendritic cells and/or macrophages. In another preferred embodiment, a pharmaceutical composition comprises a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of Gr-1⁺/CD11b⁺ myeloid progenitor cells into dendritic cells and/or macrophages.

In a specific embodiment, a pharmaceutical composition comprises a pharmaceutical carrier, one or more compounds that activate the IL-12 receptor, one or more compounds that activate the IL-3 receptor, IL-4 receptor, IL-6 receptor, Flt3, GM-CSF receptor, M-CSF receptor, G-CSF receptor, or CSF receptor, and one or more co-stimulatory molecule-activating agents. In a preferred embodiment, a pharmaceutical composition comprises a pharmaceutical carrier, one or more compounds that activate the IL-12 receptor, one or more compounds that activate the GM-CSF receptor, and one or more compounds that activate 4-1BB. In another preferred embodiment, a pharmaceutical composition comprises a pharmaceutical carrier, one or more compounds that activate the IL-12 receptor, one or more compounds that activate the GM-CSF receptor, and one or more compounds that activate OX40. In yet another preferred embodiment, a pharmaceutical composition comprises a pharmaceutical carrier, one or more compounds that activate the IL-12 receptor, one or more compounds that activate the GM-CSF receptor, one or more compounds that activate 4-1BB, and one or more compounds that activate OX40.

The present invention provides therapeutic and pharmaceutical compositions comprising a pharmaceutical carrier, one or more cytokine receptor-activating agents, and at least one fusion protein, wherein the fusion protein comprises a co-stimulatory molecule-activating polypeptide fused a heterologous protein, polypeptide or peptide. The present invention provides therapeutic and pharmaceutical compositions comprising a pharmaceutical carrier, one or more co-stimulatory molecule-activating agents, and at least one fusion protein, wherein the fusion protein comprises a cytokine receptor-activating polypeptide fused a heterologous protein, polypeptide or peptide. The present invention further provides therapeutic and pharmaceutical compositions comprising a pharmaceutical carrier and at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. Nucleic acid molecules encoding fusion proteins may be utilized in the therapeutic or pharmaceutical compositions of the invention rather than the fusion proteins themselves.

The invention also provides a pharmaceutical pack or kit comprising one or more containers with one or more of the components of the pharmaceutical compositions of the invention. The kit further comprises instructions for use of the composition. In certain embodiments of the invention, the kit comprises a document providing instructions for the use of the composition of the invention in, e.g., written and/or electronic form. Said instructions provide information relating to, e.g., dosage, methods of administration, and duration of treatment. Optionally included with such container(s) can be a notice in the form prescribed by a governmental agency regulating the manufacture, use or sale of pharmaceuticals or biological products, which notice reflects approval by the agency of manufacture, use or sale for human administration.

In accordance with the invention, any cytokine receptor-activating agent and/or co-stimulatory molecule-activating agent described herein or well-known to one of skill in the art can be incorporated in the kits of the invention. In one embodiment, a kit of the invention comprises a cytokine receptor-activating agent contained in a first vial, a co-stimulatory molecule-activating agent contained in a second vial, and instructions for administering the agents to a subject with cancer, an inflammatory disorder, or an infectious disease. In another embodiment, a kit of the invention comprises a compound that activates the IL-12 receptor contained in a first vial, a compound that activates 4-1BB contained in a second vial, and instructions for administering the compounds to a subject with cancer or an infectious disease. In another embodiment, a kit of the invention comprises a compound that activates the IL-12 receptor contained in a first vial, a compound that activates OX40 contained in a second vial, and instructions for administering the compounds to a subject with cancer or an infectious disease.

In a preferred embodiment, a kit of the invention comprises a compound that activates the IL-12 receptor contained in a first vial, a compound that activates OX40 contained in a second vial, a compound that activates 4-1BB in a third vial, and instructions for administering the compounds to a subject with cancer or an infectious disease. In another preferred embodiment, a kit of the invention comprises a compound that activates the IL-12 receptor contained in a first vial, a compound that activates the GM-CSF receptor contained in a second vial, a compound that activates 4-1BB in a third vial, and instructions for administering the compounds to a subject with cancer or an infectious disease.

3.1. Terminology

Activated immune cells: As used herein, the term “activated immune cells” refers to activated lymphoid cells (e.g., T-cells, natural killer (NK) cells, B-cells), activated myeloid cells (e.g., macrophages, monocytes, eosinophils, neutrophils, basophils, mast cells, granulocytes and platelets), activated dendritic cells, and activated antigen presenting cells. Immune cells can be determined to be activated based on the expression of specific activation markers (antigens) or the production of specific cytokines. The expression of activation markers and cytokines can be determined by a variety of methods known to those of skill in the art, including, e.g., immunofluorescence, and fluorescence activated cell-sorter (“FACS”) analysis, western blot analysis, northern blot analysis, RT-PCR.

Activated T-cells: As used herein, the term “activated T-cells” refers to T-cells expressing antigens indicative of T-cell activation (T-cell activation markers). Examples of T-cell activation markers include, but are not limited to, CD25, CD26, CD30, CD38, CD69, CD70, CD71, ICOS, OX-40 and 4-1BB. The expression of activation markers can be measured by techniques known to those of skill in the art, including, for example, western blot analysis, northern blot analysis, RT-PCR, immunofluorescence assays, and FACS analysis.

Agonistic antibodies: As used herein, the terms “agonistic antibody that immunospecifically binds to a cytokine receptor”, “agonistic antibodies that immunospecifically bind to a cytokine receptor” and analogous terms refer to antibodies that immunospecifically bind to a cytokine receptor and induce the activation of a signal transduction pathway associated with the cytokine receptor. As used herein, the terms “agonistic antibody that immunospecifically binds to a co-stimulatory molecule”, “agonistic antibodies that immunospecifically bind to a co-stimulatory molecule” and analogous terms refer to antibodies that immunospecifically bind to a co-stimulatory molecule expressed by immune cells (preferably, activated immune cells) and induce the activation of a signal transduction pathway associated with the co-stimulatory molecule. Preferably, agonistic antibodies immunospecifically bind to a co-stimulatory molecule selectively expressed by activated immune cells and augment the activation of the immune cells. More preferably, agonistic antibodies immunospecifically bind to a co-stimulatory molecule selectively expressed by activated T-cells and augment the activation of the T-cells.

Analog: As used herein, the term “analog” in the context of “an analog of a compound that activates a cytokine receptor, wherein the compound is a polypeptide (i.e., a cytokine receptor-activating polypeptide)” or “an analog of a compound that activates a co-stimulatory molecule expressed by activated immune cells, wherein the compound is a polypeptide (i.e., a co-stimulatory molecule-activating polypeptide)” refers to a polypeptide that possesses a similar or identical function as a cytokine-receptor-activating polypeptide or a co-stimulatory molecule-activating polypeptide but does not necessarily comprise: (1) a similar or identical amino acid sequence of a cytokine-receptor-activating polypeptide or a co-stimulatory molecule-activating polypeptide; or (2) or possess a similar or identical structure of a cytokine-receptor-activating polypeptide or a co-stimulatory molecule-activating polypeptide. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a cytokine-receptor-activating polypeptide or a co-stimulatory molecule-activating polypeptide; (b) a polypeptide encoded by a nucleotide sequence that hybridizes under stringent conditions to a nucleotide sequence encoding a cytokine-receptor-activating polypeptide or a co-stimulatory molecule-activating polypeptide described herein of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a cytokine-receptor-activating polypeptide or a co-stimulatory molecule-activating polypeptide. A polypeptide with a similar structure to a cytokine-receptor-activating polypeptide or a co-stimulatory molecule-activating polypeptide refers to a polypeptide that has a similar secondary, tertiary or quaternary structure to the cytokine-receptor-activating polypeptide or the co-stimulatory molecule-activating polypeptide. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the wo sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of “an analog a compound that activates a cytokine receptor, wherein the compound is not a polypeptide” or “an analog of a compound that activates a co-stimulatory molecule expressed by activated immune cells, wherein the compound is a not polypeptide “refers to an organic or inorganic compound that possesses a similar or identical function to a cytokine receptor-activating agent or a co-stimulatory molecule-activating agent and that is structurally similar to a cytokine receptor-activating agent or a co-stimulatory molecule-activating agent.

Augment: As used herein, the term “augment” in the context of augmenting activated immune cells refers to an increase in the biological activity (e.g., the proliferation, differentiation, priming, effector function, production of cytokines or expression of antigens) of activated immune cells. In particular, a compound that augments an activated T-cell activates an activated T-cell 1-5 fold, 5-10 fold, 10-20 fold or more than 20 fold as compared to the ability of the compound to activate a resting T-cell as determined by assays known to those of skill in the art, including the assays described in Section 5.8 which measure the proliferation and the expression of cytokines and antigens.

Compound that activates a cytokine receptor: As used herein, the terms “a compound that activates a cytokine receptor,” “cytokine-receptor activating agent” and analogous terms refer to agents that immunospecifically bind to or associate with one or more subunits of a cytokine receptor and induce the activation of a signal transduction pathway associated the cytokine receptor. Such agents include, but are not limited to, proteinaneous agents (e.g., cytokines, peptide mimetics, and antibodies), small molecules, organic compounds, inorganic compounds, and nucleic acid molecules encoding proteins, polypeptides, or peptides (e.g., cytokines, peptide mimetics, and antibodies) that immunospecifically bind to or associate with one or more subunits a cytokine receptor and induce the activation of a signal transduction pathway associated with the cytokine receptor. In certain embodiments, the cytokine receptor-activating agent is a protein, polypeptide, or peptide (i.e., a cytokine receptor-activating polypeptide such as a cytokine) which immunospecifically binds to or associates with one or more subunits of a cytokine receptor and induces the activation of a signal transduction pathway associated with the cytokine receptor. In other embodiments, the cytokine receptor-activating agent is a nucleic acid molecule comprising a nucleotide sequence encoding a protein, polypeptide or peptide that immunospecifically binds to or associates with one or more subunits of a cytokine receptor and induces the activation of a signal transduction pathway associated with the cytokine receptor. In certain other embodiments, the cytokine receptor-activating agent is a fusion protein or a nucleic acid molecule comprising a nucleotide sequence encoding a fusion protein, said fusion protein comprising a protein, polypeptide or peptide that immunospecifically binds to or associates with one or more subunits of a cytokine receptor and induces the activation of a signal transduction pathway associated with the cytokine receptor fused to a heterologous protein, polypeptide or peptide. In yet other embodiments, the cytokine receptor-activating agent is not fusion protein or a nucleic acid molecule comprising a nucleotide sequence encoding a fusion protein.

In a preferred embodiment, the cytokine receptor-activating agent is a cytokine, a nucleic acid molecule comprising a nucleotide sequence encoding a cytokine, an agonistic antibody which immunospecifically binds to one or more subunits of a cytokine receptor, or a nucleic acid molecule comprising a nucleotide sequence encoding an agonistic antibody that immunospecifically binds to one or more subunits of a cytokine receptor. Examples of cytokines include, but are not limited to, interferon (“IFN”)-α, IFN-β, IFN-γ, tumor necrosis actor (“TNF”)-α, Flt3 ligand, interleukin (“IL”)-1β, IL-2, IL-3, IL-4, IL-5, IL-6, IL-7, IL-8, IL-9, IL-10, IL-12, IL-15, IL-18, colony-stimulating factor (“CSF”)-1, granulocyte colony-stimulating factor (“G-CSF”), macrophage colony-stimulating factor (“M-CSF”), granulocyte macrophage colony-stimulating factor (“GM-CSF”) and chemokines such as macrophage inflammatory protein (“MIP”)-1, gamma interferon inducible protein (“IP-10”) and monokine induced by IFN-γ (“MIG”).

Cytokine receptor-activating polypeptide: As used herein, the term “cytokine receptor-activating polypeptide” and analogous terms refer to proteins, polypeptides, or peptides which immunospecifically bind to or associate with one or more subunits of a cytokine receptor and induce the activation of a signal transduction pathway associated with the cytokine receptor.

Compound that activates a co-stimulatory molecule expressed by immune cells: As used herein, the terms “a compound that activates a co-stimulatory molecule expressed by immune cells,” “co-stimulatory molecule-activating agent” and analogous terms refer to agents that immunospecifically bind to or associate with a co-stimulatory molecule expressed by an immune cell (preferably, an activated immune cell) and induce the activation of a signal transduction pathway associated the co-stimulatory molecule. In a preferred embodiment, the terms “a compound that activates a co-stimulatory molecule expressed by immune cells,” “co-stimulatory molecule-activating agent” and analogous terms refer to agents that immunospecifically bind to or associate with a co-stimulatory molecule selectively expressed by an activated immune cell (preferably, an activated T-cell, activated NK cell or activated dendritic cell) and induce the activation of a signal transduction pathway associated the co-stimulatory molecule.

Co-stimulatory molecule-activating agents include, but are not limited to, proteinaneous agents (e.g., cytokines, peptide mimetics, and antibodies), small molecules, organic compounds, inorganic compounds, and nucleic acid molecules encoding proteins, polypeptides, or peptides (e.g., cytokines, peptide mimetics, and antibodies) that immunospecifically bind to or associate with a co-stimulatory molecule expressed by an activated immune cell and induce the activation of a signal transduction pathway associated the co-stimulatory molecule. In certain embodiments, the co-stimulatory molecule-activating agent is a protein, polypeptide, or peptide (i.e., a co-stimulatory molecule-activating polypeptide) that immunospecifically binds to or associates with a co-stimulatory molecule expressed by an activated immune cell and induces the activation of a signal transduction pathway associated the co-stimulatory molecule. In other embodiments, the co-stimulatory molecule-activating agent is a nucleic acid molecule comprising a nucleotide sequence encoding a protein, polypeptide or peptide that immunospecifically binds to or associate with a co-stimulatory molecule expressed by an activated immune cell and induce the activation of a signal transduction pathway associated the co-stimulatory molecule. In certain other embodiments, the co-stimulatory molecule-activating agent is a fusion protein or a nucleic acid molecule comprising a nucleotide sequence encoding a fusion protein, said fusion protein comprising a protein, polypeptide, or peptide that immunospecifically binds to or associates with a co-stimulatory molecule expressed by an activated immune cell and induces the activation of a signal transduction pathway associated the co-stimulatory molecule fused to a heterologous protein, polypeptide or peptide. In yet other embodiments, the co-stimulatory molecule-activating agent is not fusion protein or a nucleic acid molecule comprising a nucleotide sequence encoding a fusion protein.

In a preferred embodiment, the co-stimulatory molecule-activating agent is a native or recombinant protein polypeptide, peptide, fragment, derivative or analog thereof that immunospecifically binds to a co-stimulatory molecule expressed by activated immune cells (preferably, activated T-cells), preferably a co-stimulatory molecule selectively expressed by activated immune cells (preferably, activated T-cells), and activates a signal transduction pathway associated with the co-stimulatory molecule. In another preferred embodiment, the co-stimulatory molecule-activating agent is a nucleic acid molecule comprising a nucleotide sequence encoding a protein, polypeptide, or peptide that immunospecifically binds to a co-stimulatory molecule expressed by activated T-cells, preferably a co-stimulatory molecule selectively expressed by activated T-cells, and activates a signal transduction pathway associated with the co-stimulatory molecule. In another embodiment, the co-stimulatory molecule-activating agent is a ligand for a co-stimulatory molecule (such as, e.g., SLAM, OX40, 4-1BB, CD40 ligand (CD40L), inducible co-stimulator (ICOS), B7RP-1 and CD27) expressed by activated T-cells, with the proviso that the ligand is not B7-1. Examples of such ligands, include, but are not limited to, 4-1BBL, SLAM, CD40, CD70 ligand (CD70L) and OX-40L. In another embodiment, the co-stimulatory molecule-activating agent is expressed by dendritic cells (e.g., CD40).

Co-stimulatory molecule-activating polypeptide: As used herein, the term “co-stimulatory molecule-activating polypeptide” and analogous terms refer to proteins, polypeptides, or peptides that immunospecifically bind to or associate with a co-stimulatory molecule expressed by activated immune cells (e.g., activated T-cells) and induce the activation of a signal transduction pathway associated with the co-stimulatory molecule.

Preferably, the term “co-stimulatory molecule-activating polypeptide” and analogous terms refer to proteins, polypeptides, or peptides that immunospecifically bind to or associate with a co-stimulatory molecule selectively expressed by activated immune cells (e.g., activated T-cells) and induce the activation of a signal transduction pathway associated with the co-stimulatory molecule.

Cytokine: As used herein, the term “cytokine” relates to native or recombinant secreted low molecular weight proteins, polypeptides, peptides, fragments, derivatives or analogs thereof that modulate the activity (e.g., the proliferation, differentiation and/or effector function) of immune cells. Examples of cytokines include, but are not limited to, IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, G-CSF, M-CSF, GM-CSF and chemokines such as MIP-1, IP-10 and MIG.

Derivative: As used herein, the term “derivative” in the context of a “derivative of a compound that activates a cytokine receptor, wherein the compound is a polypeptide (i.e., a cytokine receptor-activating polypeptide)” or “a derivative of a compound that activates a co-stimulatory molecule expressed by activated immune cells, wherein the compound is a polypeptide (i.e., a co-stimulatory molecule-activating polypeptide)” refers to a polypeptide that comprises an amino acid sequence of a cytokine receptor-activating polypeptide or a co-stimulatory molecule-activating polypeptide, which has been altered by the introduction of amino acid residue substitutions, deletions or additions, or by the covalent attachment of any type of molecule to the polypeptide. For example, but not by way of limitation, a cytokine receptor-activating polypeptide or a co-stimulatory molecule-activating polypeptide may be modified, e.g., by proteolytic cleavage, linkage to a cellular ligand or other protein, etc. A derivative of a cytokine receptor-activating polypeptide or a co-stimulatory molecule-activating polypeptide may be modified by chemical modifications using techniques known to those of skill in the art (e.g., by acylation, phosphorylation, carboxylation, glycosylation, selenium modification and sulfation). Further, a derivative of a cytokine receptor-activating polypeptide or a co-stimulatory molecule-activating polypeptide may contain one or more non-classical amino acids. A polypeptide derivative possesses a similar or identical function as a cytokine receptor-activating polypeptide or a co-stimulatory molecule-activating polypeptide.

As used herein, the term “derivative” in the context of a “derivative a compound that activates a cytokine receptor, wherein the compound is not a polypeptide” or “a derivative of a compound that activates a co-stimulatory molecule expressed by activated immune cells, wherein the compound is a not polypeptide” refers to an organic or inorganic compound that is formed based upon the structure of a cytokine receptor-activating agent or a co-stimulatory molecule-activating agent. A derivative of a cytokine receptor-activating agent or a co-stimulatory molecule-activating agent includes, but is not limited to, a cytokine receptor-activating agent or a co-stimulatory-activating agent that is modified, e.g., by the addition of carboxyl, amino, hydroxy or hydroxyl groups. A derivative of a cytokine receptor-activating agent or a co-stimulatory molecule-activating agent possesses a similar or identical function as the cytokine receptor-activating agent or the co-stimulatory molecule-activating agent from which it was derived.

Fragment: As used herein, the term “fragment” refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least 200 contiguous amino acid residues, or at least contiguous 250 amino acid residues of the amino acid sequence of a cytokine receptor-activating polypeptide or co-stimulatory molecule-activating polypeptide. In a specific embodiment, a fragment of a cytokine receptor-activating polypeptide retains at least one function of the cytokine receptor-activating polypeptide. In another specific embodiment, a fragment of a co-stimulatory molecule-activating polypeptide retains at least one function of the co-stimulatory molecule-activating polypeptide.

Functional fragment: As used herein, the term “functional fragment” refers to a fragment of a cytokine receptor-activating polypeptide or a co-stimulatory molecule-activating polypeptide that retains at least one function of said cytokine receptor-activating polypeptide or co-stimulatory molecule-activating polypeptide, respectively.

Fusion protein: As used herein, the term “fusion protein” refers to a polypeptide that comprises an amino acid sequence of a first protein, a functional fragment, analog or derivative thereof, and an amino acid sequence of a heterologous protein (i.e., a second protein, a functional fragment, analog or derivative thereof different than the first protein, functional fragment, analog or derivative thereof). In one embodiment, a fusion protein comprises a cytokine receptor-activating polypeptide fused to a heterologous peptide, polypeptide, or protein. In accordance with this embodiment, the heterologous peptide, polypeptide or protein may or may not be a second, different cytokine receptor-activating agent. In certain embodiments, fusion proteins used in accordance with the invention immunospecifically bind to or associate with a cytokine receptor and induce the activation of a signal transduction pathway associated with the cytokine receptor. In another embodiment, a fusion protein comprises a co-stimulatory molecule-activating polypeptide fused to a heterologous peptide, polypeptide, or protein. In accordance with this embodiment, the heterologous peptide, polypeptide or protein may or may not be a second, different co-stimulatory molecule-activating agent. In certain embodiments, fusion proteins used in accordance with the invention immunospecifically bind to or associate with a co-stimulatory molecule expressed by activated immune cells and induce the activation of a signal transduction pathway associated with the co-stimulatory molecule.

Gene products: As used herein, the term “gene products” refers to RNA molecules (e.g., mRNA), proteins, polypeptides and peptides.

Host cell: As used herein, the term “host cell” refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

Hybridizes under stringent conditions: As used herein, the term “hybridizes under stringent conditions” describes conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1× SSC, 0.2% SDS at about 68° C. In a preferred, non-limiting example stringent hybridization conditions are hybridization in 6× SSC at about 45° C., followed by one or more washes in 0.2× SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

Immunospecifically binds to an antigen: As used herein, the term “immunospecifically binds to an antigen” and analogous terms refer to peptides, polypeptides, and antibodies or fragments thereof that specifically bind to an antigen or a fragment and do not specifically bind to other antigens. A peptide or polypeptide that immunospecifically binds to an antigen may bind to other peptides or polypeptides with lower affinity as determined by, e.g., immunoassays, BIAcore, or other assays known in the art. Antibodies or fragments that immunospecifically bind to an antigen may be cross-reactive with related antigens. Preferably, antibodies or fragments that immunospecifically bind to an antigen do not cross-react with other antigens.

The term “immunospecifically binds to a cytokine receptor”, “immunospecifically binds to a co-stimulatory molecule” and analogous terms as used herein refer to peptides, polypeptides, and antibodies or fragments thereof that specifically bind to one or more subunits of a cytokine receptor or a co-stimulatory molecule and do not specifically bind to other polypeptides. A peptide or polypeptide that immunospecifically binds to one or more subunits of a cytokine receptor or a co-stimulatory molecule may bind to other peptides or polypeptides with lower affinity as determined by, e.g., immunoassays, BIAcore, or other assays known in the art. Antibodies or fragments that immunospecifically bind to one or more subunits of a cytokine receptor or a co-stimulatory molecule may be cross-reactive with related antigens. Preferably, antibodies or fragments that immunospecifically bind to a cytokine receptor or a co-stimulatory molecule do not cross-react with other antigens. Antibodies or fragments that immunospecifically bind to a cytokine receptor or co-stimulatory molecule can be identified, for example, by immunoassays, BIAcore, or other techniques known to those of skill in the art. An antibody or fragment thereof binds specifically to a cytokine receptor when it binds to a cytokine receptor with higher affinity than to any cross-reactive antigen as determined using experimental techniques, such as radioimmunoassays (RIA) and enzyme-linked immunosorbent assays (ELISAs). An antibody or fragment thereof binds specifically to a co-stimulatory molecule when it binds to a co-stimulatory molecule with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

Isolated: As used herein, an “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. Moreover, an “isolated” nucleic acid molecule can be substantially free of other cellular material, or culture medium when produced by recombinant techniques, or substantially free of chemical precursors or other chemicals when chemically synthesized. The language “substantially free of other cellular material” includes preparations of a nucleic acid molecule in which the nucleic acid molecule is separated from cellular components of the cells from which it is isolated. Thus, a nucleic acid molecule that is substantially free of cellular material includes preparations of the nucleic acid molecule having less than about 30%, 20%, 10% or 5% (by dry weight) of heterologous nucleic acid molecules. The language “substantially fee of chemical precursors or other chemical” includes preparations of a nucleic acid molecule in which the nucleic acid molecule is separated from chemical precursors or other chemicals which are involved in the synthesis of the nucleic acid molecule. Accordingly, such preparations of nucleic acid have less than about 30%, 20%, 10% or 5% (by dry weight) of chemical precursors or compounds other than the nucleic acid molecule of interest. In certain embodiments, the term “isolated”as used herein when referring to a nucleic acid molecule does not include an isolated chromosome.

An “isolated” polypeptide is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, or 5% (by dry weight) of chemical precursors or compounds other than the polypeptide of interest.

Nucleic Acids: As used herein, the terms “nucleic acids”, “nucleic acid molecules” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA. In one embodiment, the nucleotide sequences comprise a contiguous open reading frame encoding a cytokine receptor-activating polypeptide or fragment thereof, or a co-stimulatory molecule-activating polypeptide or fragment thereof, e.g., a cDNA molecule.

Prevention: As used herein, the terms “prevention of cancer”, “prevention of a tumor”, “prevent a tumor”, “preventing a tumor”, “prevent cancer” and “preventing cancer” encompass inhibiting or reducing the spread of tumor cells (metastasis), or inhibiting or reducing the onset, development or progression of one or more symptoms associated with cancer. As used herein, the terms, “prevention of an inflammatory disorder”, “preventing an inflammatory disorder”, and “prevent an inflammatory disorder” encompass inhibiting or reducing the onset, development, or progression of one or more symptoms associated with an inflammatory disorder. As used herein the terms “prevention of an infectious disease”, ” prevent an infectious disease”, and “preventing an infectious disease” encompass inhibiting or reducing the spread of the infectious agent to other tissues or subjects, or inhibiting or reducing the onset, development or progression of one or more symptoms associated with the infectious disease. Prophylactically effective amount: As used herein the term “prophylactic effective amount” refers to the amount of an agent sufficient to result in the desired prophylactic effect.

Resting T-cells: As used herein, the term “resting T-cells” refers to T-cells which do not express or express low to undetectable levels of T-cell activation markers. Resting T-cells include, but are not limited to, T-cells which are CD25⁻, CD69⁻, ICOS⁻, SLAM⁻, and 4-1BB⁻. The expression of these markers can be measured by techniques known to those of skill in the art, including, for example, western blot analysis, northern blot analysis, RT-PCR, immunofluorescence assays, and FACS analysis.

Selectively activates activated immune cells: As used herein, the term “selectively activates activated immune cells” refers to activating activated immune cells to a substantially greater degree when compared to activating resting immune cells as determined by assays known to those of skill in the art, in particular those assays described in Section 5.8. In particular, a compound that selectively activates activated immune cells refers to a compound that activates an activated immune cell 1-5 fold, 5-10 fold, 10-20 fold or more than 20 fold as compared to the ability of the compound to activate a resting immune cell as determined by assays known to those skilled in the art, including the assays described in Section 5.8 which measure the proliferation and the expression of cytokines and antigens.

Selectively activates activated T-cells: As used herein, the term “selectively activates activated T-cells” refers to activating activated T-cells to a substantially greater degree when compared to activating resting T-cells as determined by assays known to those of skill in the art, in particular those assays described in Section 5.8. In particular, a compound that selectively activates activated T-cells refers to a compound that activates an activated T-cell 1-5 fold, 5-10 fold, 10-20 fold or more than 20 fold as compared to the ability of the compound to activate a resting T-cell as determined by assays known to those skilled in the art, including the assays described in Section 5.8 which measure the proliferation and the expression of cytokines and antigens.

Selectively expressed: As used herein, the term “selectively expressed by activated immune cells” refers to molecules (e.g., co-stimulatory molecules) differentially expressed by activated immune cells relative to resting immune cells. In particular, a molecule selectively expressed by activated immune cells is expressed at 1-5 fold, 5-10 fold, 10-15 fold, 15-20 fold or more than 20 fold higher levels by activated immune cells then resting immune cells. As used herein, the term “selectively expressed by activated T-cells” refers to molecules (e.g., co-stimulatory molecules) differentially expressed by activated T-cells relative to resting T-cells. In particular, a molecule selectively expressed by activated T-cells is expressed at 1-5 fold, 5-10 fold, 10-15 fold, 15-20 fold or more 20 fold higher levels by activated T-cells than resting T-cells.

Side effects: As used herein, the term “side effects” encompasses unwanted and adverse effects of a therapeutic molecule. Adverse effects are always unwanted, but unwanted effects are not necessarily adverse. An adverse effect from a therapeutic molecule might be harmful or uncomfortable or risky. Examples of adverse side effects include, but are not limited to, fever, nausea, vomiting, the chills, and septic shock.

Subject: As used herein, the terms “subject” and “patient” refer to an animal including, but not limited to, a mammal (e.g., livestock such as a cow and a pig, a companion animal such as a cat, a dog and a horse, and a human), and a bird (e.g., a chicken). In a specific embodiment, the terms “subject” and “patient” refer to a non-human mammal. In a preferred embodiment, the terms “subject” and “patient” refer to a human. In certain embodiments, a subject or a patient is an animal, preferably a human, with cancer which is refractory to radiation or chemotherapy. In other embodiments, a subject or a patient, is an animal, preferably a human, with an inflammatory disorder which is refractory to currently used anti-inflammatory drugs. In yet other embodiments, a subject or patient is an animal, preferably a human, with an infectious disease which refractory to currently used antibiotics or anti-viral agents.

Therapeutically effective amount: As used herein, the terms “therapeutically effective amount” and “an effective amount” refer to the amount of an agent sufficient to result in the desired therapeutic effect. With regard to the treatment of cancer, the terms “therapeutically effective amount” and “an effective amount” refer to the amount of one or more cytokine receptor-activating agents and the amount of one or more co-stimulatory molecule-activating agents sufficient to inhibit or reduce the growth of a tumor or tumor cells, reduce the volume of a tumor, kill tumor cells, inhibit or reduce the spread of tumor cells (metastasis), or ameliorate one or more symptoms associated with a cancer. With regard to the treatment of an inflammatory disorder, the terms “therapeutically effective amount” and “an effective amount” refer to the amount of one or more cytokine receptor-activating agents and the amount of one or more co-stimulatory molecule-activating agents sufficient to reduce the inflammation of a particular tissue and/or joint, or ameliorate one or more symptoms associated with the inflammatory disorder. With regard to infectious diseases, the terms “therapeutically effective amount” and “an effective amount” refer to the amount of one or more cytokine receptor-activating agents and the amount of one or more co-stimulatory molecule-activating agents sufficient to reduce or inhibit the replication of an infectious agent (e.g., bacteria, viruses, or fungi), kill the infectious agent, inhibit or reduce the spread of the infectious agent to other tissues or subjects, or ameliorate one or more symptoms associated with the infectious disease. In certain embodiments, the terms “therapeutically effective amount” and “an effective amount” refer to the amount of one or more cytokine receptor-activating agents and the amount of one or more co-stimulatory molecule-activating agents sufficient to augment the activation of immune cells (e.g., T-cells and NK cells), augment the differentiation of myeloid cells into dendritic cells or macrophages, or augment the immune response.

Treatment: As used herein, the terms “treatment of cancer”, “treatment of a tumor”, “treat a tumor”, “treating a tumor”, “treat cancer” and “treating cancer” encompass inhibiting or reducing the growth of a tumor or tumor cells, reducing the volume of a tumor, killing tumor cells, inhibiting or reducing the spread of tumor cells (metastasis), or ameliorating one or more symptoms associated with cancer. As used herein, the terms, “treatment of an inflammatory disorder”, “treating an inflammatory disorder”, and “treat an inflammatory disorder” encompass reducing the inflammation of tissues and/or joints of a subject or ameliorating one or more symptoms associated with an inflammatory disorder. As used herein the terms “treatment of an infectious disease”, “treat an infectious disease”, and “treating an infectious disease” encompass reducing or inhibiting the replication of an infectious agent (e.g., bacteria, viruses, or fungi), killing the infectious agent, inhibiting or reducing the spread of the infectious agent to other tissues or subjects, or ameliorating one or more symptoms associated with the infectious disease.

### 4. DESCRIPTION OF THE FIGURES

FIG. 1. Survival of tumor bearing animals after ADV.mIL-12 and anti-41BB treatment. Animals were intrahepatically implanted with 7×10⁴ MCA26 tumor cells for 7 days. The animals with hepatic tumor sizes of 5×5 mm² were divided into several groups. the groups were injected intratumorally with various doses of Adv.mIL-12 (3.2×10⁸ pfu, n=8; 1.6×10⁸ pfu, n=8; 0.8×10⁸ pfu; n=12; 0.4×10⁸ pfu, n=5; 0.2×10⁸ pfu, n=5; and 0.1×10⁸ pfu, n=5) or a control vector, DL312 (3.2×10⁸ pfu, n=12) in combination with anti-4-1BB or a control antibody. The antibodies were injected intraperitoneally at days 8 and 11 at a does of 50% g/mouse. Survival difference between combination IL-12 (3.6×10⁸pfu)+anti-4-1BB treated animals was statistically significant from either DL312+anti-4-1BB (n=12) or (ADV.mIL-12+control Ig (3.2×10⁸ pfu, n=12) treated animals by Logrank survival analysis (p<0.0001). The results reported here were pooled from two consecutive sets of experiments.

FIG. 2. Long-term survival study of BALB/c mice bearing JC breast carcinoma liver metastases treated with ADV/IL-12+anti-4-1BB antibody. Animals bearing tumors 5×5 mm in diameter were attributed to four groups (n=15-25 animals/group): 1) (♦) ADV/IL-12 (1×10⁸ pfu/animal)+anti-4-1BB (2×50 μg i.p.); 2) (▴) ADV/IL-12 (3.6×10⁸ pfu/animal); 3) (▪) ADV/D1312+anti-4-1BB; 4) (X) ADV/DL312+control Ig. 87% of the combination IL-12 plus anti-4-1BB treated animals showed long-term survival while 60% of the anti-4-1BB treated animals did so (P=0.02, logrank test). In the IL-12 group, 22% of the mice survived while 100% of the control animals died within 60 to 70 days after tumor inoculation.

FIG. 3. Combination adenoviral mediated gene therapy of IL-12 and 4-1BB ligand. Animals with hepatic tumor at size 5×5 mm² were divided into four groups, and each group (n=5-7) were injected intratumorally with various doses of Adv.m4-1BB ligand (1×10⁹ and 0.5×10⁶ pfu) in combination with Adv.mIL-12 (2×10⁸ pfu) or control vector, DL312 (2×10⁸ pfu). The survival difference between the combination of IL-12 and 4-1BB ligand treated animals was statistically significant than either Adv.m4-1BB ligand and DL312 or Adv.mIL-12 and DL312 treated animals by Logrank survival analysis (p<0.042).

FIG. 4. Long-term survival study of BALB/c mice bearing JC breast carcinoma liver metastases treated with ADV/IL-12+ADV/4-1BBL. Animals bearing tumors 5×5 mm in diameter were attributed to four groups: 1) (♦) ADV/IL-12 (1×10⁸ pfu/animal)+ADV/4-1BBL (1×10⁹ pfu/animal); 2) (▪) ADV/IL-12 (1×10⁸ pfu/animal)+ADV/DL312 (1×10⁹ pfu/animal); 3) (▴) ADV/D 1312+anti-4-1BB (1×10⁹ pfu/animal); 4) (X) ADV/DL312 (1.1×10⁹ pfu/animal). 78% of combination IL-12+4-1BBL treated animals showed long-term survival. Compared to IL-12 (22% survival) or 4-1BBL (13% survival) alone, the difference is statistically significant with P values of 0.016 and 0.004, respectively (logrank test).

FIG. 5. Subcutaneous challenge of long-term surviving animals after JC liver metastases treatment. Surviving (>120 days after tumor cell inoculation) animals after treatment with ADV/IL-12 or anti-4-1BB antibody alone, or combination ADV/IL-12+anti-4-1BBL received a s.c. injection of JC parental cells or MCA26 cells. Formation of tumor was observed over a 4-week period. Naive animals were also injected to assess the normal growth pattern of the 2 tumors. Various percentages of animals in the long-term surviving groups did not form any tumor. However, only the results of the ADV/IL-12+ADV/4-1BBL group reached statistical significance compared to naive controls (P=0.007, Fischer's exact test). Conversely, the rate of JC tumor growth was dramatically reduced among all surviving animals.

FIG. 6. Effect of hepatic tumor combination treatment on macroscopic lung metastases of colon carcinoma. An animal model with both liver tumor and pre-established multiple macroscopic tumor nodules in the lung was subjected to a test for the systemic anti-tumor effect. Control animals receiving no treatment developed multiple lesions in the lung, and all of them died within 32 days. 100% of the liver and lung tumor bearing animals receiving the combination treatment (0.4×10⁸pfu Adv. mIL-12+anti 4-1BB) in the liver tumor (n=6) survived well after 70 days. The results indicate distant protection against pre-existing macroscopic lung metastases by hepatic tumor combination treatment (p<0.001 1) by Logrank test.

FIG. 7. (A) Evaluation of cellular immune response in Adv.mIL-12 (0.4×10⁸pfu) and anti-4-1BB treated animals. MNC were isolated from animals at days 0, 1, 2, 4, 7 and 14 (five mice per time point per group) after treatments and the cells were assayed for direct cytolytic killing against ⁵¹Cr labeled parental MCA26 tumor cells. Direct tumor cell killing activity can be seen at days 2 and 4 in ADV.mIL-12+anti-4-1BB treated animals, and only low activity was present in anti-4-1-BB alone or ADV.mIL-12 alone treated animals. The standard deviation of the triplicate wells is less than 7% (B) Identification of effector cell types by in vitro depletion of effector cells. MNC isolated from combination treated animals at day 2 were divided and depleted of NK, CD4+T or pan-T cells, using purified D×5 antibody, GK1.5 and Thyl.2 hybridoma supernatant, respectively, that were conjugated with complement. The control group was treated with complement alone. Less than 1% of T cells remained after depletion as confirmed by FACS staining, and less than 5% of NK activity remained as confirmed by YAC-1 killing. The standard deviation of the triplicate wells is usually less than 7%.

FIG. 8. Effect of leukocyte depletion on tumor rechallenge in long-term surviving animals. Long-term surviving animals were depleted of NK (n=8) cells at optimal conditions and with appropriate controls, including non-tumor bearing naive (n=8) and control Ig (n=7), prior to being challenged by subcutaneous injection of parental MCA26 tumor cells (7×10⁸). Over a four-week observation period, 100% of the non-tumor bearing native animals formed subcutaneous tumor, and only 14.2% of control Ig injected mice formed tumor. In the NK deleted group, 87.5% of the animals formed MCA26 tumor, and 100% of the CD8+T cell depleted animals formed tumor. (*) indicates statistical significance when compared to control Ig treated group by Fisher Exact test.

FIG. 9. Survival of tumor-bearing mice after Adv.mIL-12, anti-4-1BB antibody, and anti-OX40 antibody treatment. MCA26 (9×10⁴) were implanted into the liver of syngeneic BALB/c mice. After 9 days, mice with hepatic tumors (8×8 to 11×11 mm² in diameter) were randomly assigned to the following groups: (1) Adv.mIL-12, anti-4-1BB antibody and anti-OX40 antibody (n=33); (2) Adv.mIL-12, anti-4-lBB antibody and rat Ig (n=32); (3) DL312, anti-4-1BB antibody and anti-OX40 antibody (n=7); (4) Adv.mIL-12, rat Ig and anti-OX40 antibody (n=12); (5) DL312, anti-4-1BB antibody and rat Ig (n=4); (6) DL312, rat Ig, and rat Ig (n=12); and (7) DL312, rat Ig, and anti-OX40 antibody (n=4). Anti-4-1BB antibody or control rat Ig and anti-OX40 antibody or control rat Ig were given i.p. at days 10 and 12 and days 11 and 13, respectively. The survival advantage for the mice reated with IL-12, anti-4-1BB antibody and anti-OX40 antibody was statistically significant compared to the IL-12 and anti-4-1BB antibody treated mice (p=0.03, log-rank test). The results were combined from three consecutive sets of experiments.

FIG. 10. Cytotoxic activity against MCA26 cells by tumor infiltrating leukocytes (TILs) isolated from mice treated with Adv.mIL-12, anti-4-1BB antibody and anti-OX40 antibody combination therapy. Ex vivo tumor cytolysis by TILs was evaluated at day 9 after Adv.mIL-12 injection. TILs were isolated from 3 mice per group and used in a standard 4 hour ⁵¹Cr release assay without in vitro stimulation. The results shown are from 3 independent experiments. TILs isolated from mice treated with Adv.mIL-12, anti-4-1BB antibody and anti-OX40 antibody combination therapy exhibit a significantly higher cytotoxic activity against MCA26 cells than those isolated from Adv.mIL-12 and anti-4-1BB antibody treated mice at all E/T ratios tested (only E/T=50 is shown, p=0.029). The CTL activity was completely inhibited by pre-incubation of TILs with anti-CD3 monoclonal antibodies.

FIGS. 11A-11B. The effect of in vivo CD4 depletion on the CD8⁺T-cells in tumor infiltrating leukocytes (TILs) and the CTL response. Mononuclear cells were isolated and combined from 3 mice per treatment group at day 9 after treatment and used in flow cytometric analysis and the cytotoxic assay. Data are representative of two experiments: (A) Flow cytometric analysis of TILs. Isolated TILs stained with FITC conjugated anti-CD4 antibody and PE conjugated anti-CD8 antibody were analyzed on a FACScan flow cytometer. A higher number of CD8⁺T-cells was observed in the TILs isolated from mice treated with the Adv.mIL-12, anti-4-1BB antibody and anti-OX40 antibody combination therapy when compared to those from Adv.mIL-12 and anti-4-1BB antibody treated mice. In CD4 depleted mice treated with the Adv.mIL-12, anti-4-1BB antibody and anti-OX40 antibody combination therapy, a decrease in CD8⁺ cells was observed as compared to the control group. (B) Ex vivo tumor lysis by TILs. Isolated TILs were assayed for direct cytolysis against ⁵¹Cr-labeled parental MCA26 tumor target. Higher direct cytolysis activity by TILs was observed in mice treated with the Adv.mIL-12, anti-4-1BB antibody and anti-OX40 antibody combination therapy when compared to Adv.mIL-12 and anti-4-1BB antibody treated mice (p<0.01 at all E/T ratios tested). With in vivo CD4 depletion, the TIL direct CTL activity of mice treated the Adv.mIL-12, anti-4-1BB antibody and anti-OX40 antibody combination therapy decreased to a level similar to that of Adv.mIL-12 and anti-4-1BB antibody treated mice (p<0.01 at all E/T ratios tested).

FIG. 12. Memory CTL response against MCA26 cells by splenocytes isolated from long-term surviving mice. Tumor lysis against parental MCA26 cells was performed on individual mice cured of hepatic tumor at 120 days after treatment using a 4 hour ⁵¹Cr release assay. Results from 6 independent experiments are shown. The splenocytes were isolated from tumor-free long-term surviving mice and co-cultured with irradiated MCA26 cells in the presence of 20 U/ml murine recombinant IL-2 for 5 days before performing the CTL assay. Multiple E/T ratios were tested, but only the results for an E/T ratio of 6.5 are presented. In vitro blocking with anti-CD3 monoclonal antibodies completely abolished the cytolytic activity in both treatment groups. Mice treated with the Adv.mIL-12, anti-4-1BB antibody and anti-OX40 antibody combination therapy exhibited significantly higher CTL activities as compared to those treated with Adv.mIL-12 and anti-4-1BB antibody (p=0.0001).

FIGS. 13A-13C. Fraction II (Fril) of Percoll gradient derived from bone marrow (BM) or spleen of MCA-26 tumor-bearing mice inhibits the CD3/CD28-induced T-cell proliferative response. Cells from low density Fr.II (50-60%, 1.063-1.075g/ml) or Fr.III (60-70%, 1.075-1.090), obtained by Percoll fractionation from BM of naive mice (A) or BM or spleen of tumor-bearers (B, C), were added (2×10⁵/well) to naive splenocytes (2×10⁵/well) in the presence of CD3 (1 μg/ml) alone or in combination with CD28 (5 μg/ml) monoclonal antibodies (mAbs). Cells were co-cultured for 72 hours and incorporation of ³H-thymidine was measured. The results shown are the average of triplicates and representative of four separate experiments. White bars—naïve splenocytes only; black bars—naive splenocytes plus cell Fr. II; hatched bars—naive splenocytes plus cell Fr. III.

FIG. 14. Comparative inhibitory activity of Fr.II cells derived from BM of naive and tumor-bearing (TB) mice. Freshly isolated BM cells were fractionated on a Percoll gradient. A graded number of Fr.II cells (0.5-2×10⁵/well) were added to naive splenocytes (2×10⁵/well) activated with CD3 (1 μg/ml) and CD28 (5 μg/ml) mAbs. Cells were co-cultured for 72 hours and incorporation of ³H-thymidine was measured. Results presented are the average of triplicates and representative of three such experiments.

FIGS. 15A-15B. Involvement of reactive nitrogen and oxygen species in mechanisms of immune suppression. (A) Comparative levels of nitrites in cell culture supernatants. T-cell activation assays were set up in the presence or absence of Fr.II cells derived from spleen or BM of naive or tumor-bearing mice. Cells were co-cultured for 72 hours using a 1:1 cell ratio and culture supernatants were collected. The amount of NO secreted into the culture supernatant was detected using Greiss reagent. Results presented are the average of triplicates and representative of five separate experiments. (B) Reversal of immune suppression in the presence of L-NMMA and MnTBAP. T-cell proliferation assays were set up in the presence or absence of Fr.II cells derived from spleen or BM of tumor-bearing mice. A combination of L-NMMA (0.5 mM) and MnTBAP (10 μM) was added to the cultures. Cells were co-cultured using a 1:1 cell ratio for 72 hours and the incorporation of ³H-thymidine was measured. Results presented are the average of triplicates and representative of two separate experiments.

FIGS. 16A-16B. Inhibitory myeloid progenitor cells can inhibit the proliferative response of HA-TCR transgenic CD4 T cells induced by HA peptides. (A) BM cells were derived from MCA-26 tumor bearing mice and fractionated on a Percoll gradient. Cells from Fr.II, or Fr.III (0.5×10⁵/well) were added to the transgenic splenocytes (2×10⁵/well) with various concentrations of HA peptide (μg/ml). Cells were co-cultured for 72 hours. Results presented are the average of triplicates and representative of two separate experiments. (B) Another plate set up under the same conditions was used for the measurement of nitrite accumulation. The culture supernatants were collected. The amount of NO secreted into the culture supernatant was detected using Greiss reagent. Results presented are the average of triplicates and representative of two separate experiments.

FIG. 17. Flow cytometric analysis of BM Fr.II cells isolated from naive and MCA26 large tumor bearing mice enriched by a percoll gradient. The cells were stained with FITC-conjugated anti-CD31, Ly6C, and PE conjugated anti-CD40, Gr-1 and Class II (I-A/I-E). Conjugated isotype-matched mAbs were used as a control. The results are presented as % of positive cells. The staining results are an average from three separate experiments. * represents the statistically significant difference.

FIG. 18. Inhibitory myeloid progenitor cells from JC breast tumor bearing animals can inhibit the proliferative response of HA-TCR transgenic CD8 T cells induced by HA peptides. BM cells derived from JC breast tumor bearing mice were fractionated on a percoll gradient as requested by reviewer to demonstrate that the inhibition effect is also present in other tumor model. Various cell ratios from Fr.II, or F4/80 depleted Fr.II cells were co-cultured with the transgenic splenocytes (2×10⁵/well) in the presence of CD8 HA peptide (4 g/ml) for 72 h. Results presented are the average of triplicates.

FIG. 19. Immunostaining of DCs generated from different culture conditions. BM Fr.II cells derived from naive and MCA-26 tumor bearing mice were cultured with mGM-CSF for 10 days. Non-adherent cells were harvested and cultured in the presence of mGM-CSF or mGM-CSF and anti-CD40 mAb (5 mg/ml, FGK45 clone) for additional 24 hours. Non-adherent cells were stained and analyzed for the expression of various surface molecules by flow cytometry. Mean±SD is obtained from three independent experiments.

FIGS. 20A-20B. Increase of CD11c⁺ dendritic cells (DCs) infiltrating at the tumor site in ADV/mGM-CSF-treated mouse in vivo. Seven days after injection of ADV/mGM-CSF (4.4×10⁹ pfu/mouse) or control vector, DL-312, into MCA-26 tumor bearing mouse by direct intratumor injection. TILs were isolated and stained for Gr-1-PE, Ly-6C-FITC and biotinylated-CD11c followed by streptavidin-APC. CD11c⁺ cells were gated on Gr-1⁺/Ly-6C⁺ cells. Histogram depicts the relative fluorescence of a representative TIL sample from (A) ADV/mGM-CSF and (B) control vector, DL-312, injected mice.

FIGS. 21A-21D. Effect of ADV/mGM-CSF on the induction of CD11c⁺/I-A/I-E⁺ DCs. Recipient MCA-26 tumor bearing mouse received 4.4×10⁹ pfu/mouse of ADV/mGM-CSF (B and D) or DL312, control vector (A and C) by intratumoral injection. 24 hours later, BM and spleen Fr.II cells were labeled with CFSE (10 μM) and adoptive transfer to recipient. Each recipient mouse received 2×10⁷ CFSE-labeled Fr.II cells by tail vein infusion. Splenocytes were isolated 5 days after adoptive transfer and stained with PE-CD11c and biotinylated-MHC II (I-A/I-E) or isotype controls. (A and B) Relationship between cell division and expression of DCs markers CD11c and I-A/I-E within myeloid progenitor Fr.II cells. The y-axis represents the fluorescent intensity of CD11c⁺ cells; the x-axis represents green fluorescence intensity due to CFSE-labeling. (C and D) The expression of CD11c and I-A/I-E on gated CFSE positive daughter cells. The numbers represent the percentage of double positive cells.

FIG. 22. Long-term survival of tumor-bearing mice receiving ADV/GM-CSF in conjunction of IL-12 and anti4-1BB monoclonal antibody. Mice bearing 5×6 mm² tumors were injected with ADV/GM-CSF (n=10) or control DL-312 virus (n=10) or buffer alone (n=10). Eight days after the GM-CSF injection, mice bearing tumors larger than 10 mm2 were treated with the ADV/IL-12 virus and anti4-1BB monoclonal antibody. The long-term survival of these mice were assessed.

### 5. DETAILED DESCRIPTION OF THE INVENTION

The present invention provides methods for preventing or treating cancer, an inflammatory disorder or an infectious disease in a subject comprising administering to a subject in need thereof an effective amount one or more compounds that activate one or more cytokine receptors (i.e., one or more cytokine receptor-activating agents) and an effective amount of one or more compounds that activate one or more co-stimulatory molecules expressed by activated immune cells, preferably activated T-cells (i.e., co-stimulatory molecule-activating agents). In particular, the present invention provides methods for treating or preventing cancer, an inflammatory disorder or an infectious disease in a subject comprising administering to a subject in need thereof an effective amount of one or more cytokine receptor-activating agents) and an effective amount of one or more compounds that activate one or more co-stimulatory molecules selectively expressed by activated immune cells, preferably activated T-cells.

The cytokine receptor-activating molecules used in accordance with the methods of the invention may be proteinaneous agents (e.g., cytokines, peptide mimetics, and antibodies), small molecules, organic compounds, inorganic compounds, or nucleic acid molecules encoding proteins, polypeptides, or peptides (e.g., cytokines, peptide mimetics, and antibodies) that immunospecifically bind to or associate with one or more subunits of a cytokine receptor and induce the activation of a signal transduction pathway associated the cytokine receptor. The co-stimulatory molecule-activating agents used in accordance with the methods of the invention may be proteinaneous agents (e.g., cytokines, peptide mimetics, and antibodies), small molecules, organic compounds, inorganic compounds, or nucleic acid molecules encoding proteins, polypeptides, or peptides (e.g., cytokines, peptide mimetics, and antibodies) that immunospecifically bind to or associate with a co-stimulatory molecule expressed by an immune cell (preferably, an activated immune cell) and induce the activation of a signal transduction pathway associated the co-stimulatory molecule. Preferably, the co-stimulatory molecule-activating agents used in accordance with the methods of the invention immunospecifically bind to and induce the activation of a signal transduction pathway associated with a co-stimulatory molecule selectively expressed by activated by activated T-cells.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, and an effective amount of one or more co-stimulatory molecule-activating agents. Preferably, the cytokine receptor-activating agent shifts the Th1/Th2 balance in a subject, and more preferably, the cytokine receptor-activating agent shifts the Th1/Th2 balance and induces the proliferation and/or differentiation of Th1 cells in a subject. In particular, the present invention provides methods for preventing or treating cancer or an infectious disease in a subject, said methods comprising administering to a subject in need thereof an effective amount of one or more compounds that activates the IL-12 receptor (e.g., IL-12 or anti-IL-12R antibodies) and an effective amount of one or more co-stimulatory molecule-activating agents (e.g., OX40L, anti-OX40 antibodies, 4-1BB ligand and/or anti-4-1BB antibody).

The present invention provides methods for preventing or treating cancer or an infectious disease in a subject, said methods comprising administering to a subject in need thereof an effective amount of one or more compounds that activate the IL-12 receptor, an effective amount of one or more compounds that activate at least one cytokine receptor other than the IL-12 receptor, and an effective amount of one or more co-stimulatory molecule-activating agents. The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, and an effective amount of one or more co-stimulatory molecule-activating agents which affect the biological activity (e.g., differentiation, proliferation or effector function) of dendritic cells and/or macrophages.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, an effective amount of one or more cytokine receptor-activating agents which promote the differentiation of myeloid cells into dendritic cells and/or macrophages, and an effective amount of one or more co-stimulatory molecule-activating agents which affect the biological activity (e.g., differentiation, proliferation or effector function) of dendritic cells and/or macrophages. The present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells/NK cells, and an effective amount of one or more cytokine receptor-activating agents which promote the differentiation of myeloid cells into dendritic cells and/or macrophages. In particular, the present invention provides methods for treating or preventing cancer or an infectious disease in a subject, said methods comprising administering to a subject in need thereof an effective amount of one or more compounds that activate the IL-12 receptor, an effective amount of one or more compounds that activate the GM-CSF receptor, and an effective amount of one or more co-stimulatory molecule-activating agents (e.g., OX40L, anti-OX40 antibody, 4-1BB ligand and/or anti-4-1BB antibody).

The present invention provides methods for preventing or treating cancer, an inflammatory disorder, or an infectious disease in a subject, said methods comprising administering to a subject in need thereof an effective amount of one or more cytokine receptor-activating agents and an effective amount of at least one fusion protein, wherein the fusion protein comprises a co-stimulatory molecule-activating polypeptide fused a heterologous protein, polypeptide or peptide. The present invention also provides methods for preventing or treating cancer, an inflammatory disorder, or an infectious disease in a subject, said methods comprising administering to a subject in need thereof an effective amount of one or more co-stimulatory molecule-activating agents and an effective amount of at least one fusion protein, wherein the fusion protein comprises a cytokine receptor-activating polypeptide fused a heterologous protein, polypeptide or peptide. The present invention further provides methods for preventing or treating cancer, an inflammatory disorder, or an infectious disease in a subject, said methods comprising administering to a subject in need thereof an effective amount of at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. Nucleic acid molecules encoding fusion proteins may be administered to a subject with cancer, an inflammatory disorder or an infectious disease rather than the fusion proteins themselves.

In accordance with the methods of the invention, one or more cytokine receptor-activating agents may be administered to a subject with cancer, an inflammatory disorder or an infectious disease prior to, concomitantly with, or subsequent to the administration of one or more co-stimulatory molecule-activating agents. Further, in accordance with the methods of the invention, a subject with cancer, an inflammatory disorder or an infectious disease may be administered repeated doses of cytokine receptor-activating agents and/or co-stimulatory molecule-activating agents as part of a therapeutic protocol to treat cancer, an inflammatory disorder or an infectious disease. The cytokine receptor-activating agents and/or co-stimulatory molecule-activating agents may be administered locally and/or systemically. In specific embodiments of the invention, the cytokine receptor-activating agents and the co-stimulatory molecule-activating agents may be administered separately or as an admixture.

The methods of the invention provide a better therapeutic effect than currently existing clinical therapies for cancers, inflammatory disorders, and infectious diseases. The methods of the present invention enable lower dosages and/or less frequent dosing of cytokine receptor-activating agents (e.g., IL-12 and/or GM-CSF) and/or co-stimulatory molecule-activating agents (e.g., anti-4-1BB antibody and/or anti-OX40 antibody) to be administered to a subject with cancer, an inflammatory disorder or an infectious disease to achieve a therapeutic effect. The methods of the invention also reduce or avoid the adverse or unwanted side effects associated with the administration of cytokine receptor-activating agents and/or co-stimulatory molecule-activating agents.

The invention provides therapeutic and pharmaceutical compositions comprising pharmaceutically acceptable carriers, one or more cytokine receptor-activating agents, and one or more co-stimulatory molecule-activating agents. The present invention also provides therapeutic or pharmaceutical compositions comprising a pharmaceutical carrier, one or more cytokine receptor-activating agents which affect the biological activity (e.g., differentiation, proliferation or effector function) of T helper (Th) cells and/or NK cells, and one or more co-stimulatory molecule-activating agents. The present invention further provides therapeutic or pharmaceutical compositions comprising a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of myeloid cells into dendritic cells and/or macrophages.

The present invention provides pharmaceutical compositions comprising a pharmaceutical carrier, one or more cytokine receptor-activating agents, and at least one fusion protein, wherein the fusion protein comprises a co-stimulatory molecule-activating polypeptide fused a heterologous protein, polypeptide or peptide. The present invention also provides pharmaceutical compositions comprising a pharmaceutical carrier, one or more co-stimulatory molecule-activating agents, and at least one fusion protein, wherein the fusion protein comprises a cytokine receptor-activating polypeptide fused a heterologous protein, polypeptide or peptide. The present invention further provides pharmaceutical compositions comprising a pharmaceutical carrier and at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. Nucleic acid molecules encoding fusion proteins may be utilized in the pharmaceutical compositions of the invention rather than the fusion proteins themselves.

The pharmaceutical compositions of the invention may be used in accordance with the methods of the invention for the treatment of cancer, an inflammatory disorder, or an infectious disease in a subject. The pharmaceutical compositions of the present invention are in suitable formulation to be administered to animals, preferably mammals such as companion animals (e.g., dogs, cats, and horses) and livestock (e.g., cows and pigs), and most preferably humans. In accordance with the invention, cytokine receptor-activating polypeptides and/or co-stimulatory molecule-activating polypeptides can be supplied by direct administration or indirectly as “pro-drugs” using somatic cell gene therapy.

The invention provides pharmaceutical packs or kits comprising one or more containers one or more cytokine receptor-activating agents and one or more co-stimulatory molecule-activating agents. Preferably, the kit further comprises instructions for use of the agents. In certain embodiments of the invention, the kit comprises a document providing instructions for the use of the agents in, e.g., written and/or electronic form. Said instructions provide information relating to, e.g., dosage, methods of administration, and duration of treatment. Optionally included with such container(s) can be a notice in the form prescribed by a governmental agency regulating the manufacture, use or sale of pharmaceuticals or biological products, which notice reflects approval by the agency of manufacture, use or sale for human administration.

5.1. Cytokine Receptor-Activating Agents

Any compound well-known to one of skill in the art that immunospecifically binds or associates with one or more subunits of a cytokine receptor and induces the activation of a signal transduction pathway associated the cytokine receptor (i.e., a cytokine receptor-activating agent) may be used in the methods and compositions of the invention. Cytokine receptor-activating agents include, but are not limited to, proteinaneous agents (e.g., cytokines, peptide mimetics, and antibodies), small molecules, organic compounds, inorganic compounds, and nucleic acid molecules comprising nucleotide sequences encoding proteins, polypeptides, or peptides (e.g., cytokines, peptide mimetics, and antibodies) that immunospecifically bind to or associate with one or more subunits of a cytokine receptor and induce the activation of a signal transduction pathway associated with the cytokine receptor.

In certain embodiments, the cytokine receptor-activating agent is a protein, polypeptide, or peptide (i.e., a cytokine receptor-activating polypeptide such as a cytokine) that immunospecifically binds to or associates with one or more subunits of a cytokine receptor and induces the activation of a signal transduction pathway associated with the cytokine receptor. In other embodiments, the cytokine receptor-activating agent is a nucleic acid molecule comprising a nucleotide sequence encoding a protein, polypeptide or peptide that immunospecifically binds to or associates with one or more subunits of a cytokine receptor and induces the activation of a signal transduction pathway associated with the cytokine receptor. In certain other embodiments, the cytokine receptor-activating agent is a fusion protein or a nucleic acid molecule comprising a nucleotide sequence encoding a fusion protein, said fusion protein comprising a protein, polypeptide or peptide that immunospecifically binds to or associates with one or more subunits of a cytokine receptor and induces the activation of a signal transduction pathway associated with the cytokine receptor fused to a heterologous protein, polypeptide or peptide. In yet other embodiments, the cytokine receptor-activating agent is not fusion protein or a nucleic acid molecule comprising a nucleotide sequence encoding a fusion protein.

In a preferred embodiments, the cytokine receptor-activating agent is a cytokine, a nucleic acid molecule comprising a nucleotide sequence encoding a cytokine, an agonistic antibody which immunospecifically binds to a cytokine receptor, or a nucleic acid molecule comprising a nucleotide sequence encoding an agonistic antibody that immunospecifically binds to a cytokine receptor. Examples of cytokines include, but are not limited to, IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, CSF-1, G-CSF, M-CSF, GM-CSF and chemokines such as MIP-1, IP-10 and MIG. Examples of antibodies include, but are not limited to, antibodies that immunospecifically bind to the IFN-α receptor, IFN-β receptor, IFN-γ receptor, TNF-α receptor, Flt3, IL-1β, receptor, IL-2 receptor, IL-3 receptor, IL-4 receptor, IL-5 receptor, IL-6 receptor, IL-7 receptor, IL-8 receptor, IL-9 receptor, IL-10 receptor, IL-12 receptor, IL-15 receptor, IL-18 receptor, CSF-1 receptor, G-CSF receptor, M-CSF receptor, GM-CSF receptor, MIP-1 receptor, IP-10 receptor and MIG receptor.

5.1.1. Cytokines

The present invention encompasses the use of one or more cytokines and one or more nucleic acid molecules comprising nucleotide sequences encoding one or more cytokines in the compositions, kits and methods of the invention. The nucleotide sequence and/or amino acid sequences of cytokines can be obtained, e.g., from the literature or a database such as GenBank. For example, the nucleotide sequences of human IL-12, human IL-15, human IL-18, and human GM-CSF can be found in GenBank under GenBank Access Nos. AF050083, X94222, E17135 and E00951, respectively. In a preferred embodiment, one or more cytokines, or one or more nucleic acid molecules comprising nucleotide sequences encoding one or more cytokines that alter the biological activity of Th1 and/or Th2 cells are utilized in the compositions, kits and methods of the invention. In another preferred embodiment, one or more cytokines, or one or more nucleic acid molecules comprising nucleotide sequences encoding one or more cytokines that alter the biological activity of NK cells are utilized in the compositions, kits and methods of the invention.

In another preferred embodiment, one or more cytokines, or one or more nucleic acid molecules comprising nucleotide sequences encoding one or more cytokines that alter the biological activity of dendritic cells are utilized in the compositions, kits and methods of the invention. In another preferred embodiment, one or more cytokines, or one or more nucleic acid molecules encoding one or more cytokines that promote the differentiation of myeloid cells into dendritic cells and/or macrophages are utilized in the compositions, kits and methods of the invention. In another preferred embodiment, one or more cytokines, or one or more nucleic acid molecules encoding one or more cytokines that promote the differentiation of Gr-1⁺ myeloid progenitor cells into dendritic cells and/or macrophages are utilized in the compositions, kits and methods of the invention. In yet another preferred embodiment, one or more cytokines, or one or more nucleic acid molecules encoding one or more cytokines that promote the differentiation of Gr-1⁺/CD11b⁺ myeloid progenitor cells into dendritic cells and/or macrophages are utilized in the compositions, kits and methods of the invention. Examples of cytokines that promote the differentiation of myeloid cells into dendritic cells and/or macrophages include, but are not limited to, IL-3, IL-4, IL-6, Flt-3 ligand, GM-CSF, M-CSF, G-CSF, and CSF. In a particular embodiment, IL-2, IL-3, IL-4, IL-6, IL-12, IL-15, IL-18, M-CSF, G-CSF, CSF, Flt3 ligand, and/or GM-CSF are used in the compositions, kits and methods of the invention.

The present invention encompasses the use of fragments, derivatives and analogs of cytokines that immunospecifically bind to one or more subunits of a cytokine receptor in the compositions, kits and methods of the invention. Preferably, fragments, derivatives and analogs of cytokines retain the ability to immunospecifically bind to one or more subunits of a cytokine receptor and induce the activation of a signal transduction pathway associated with the cytokine receptor. Cytokines, and fragments, derivatives and analogs thereof that immunospecifically bind to one or more subunits a cytokine receptor can be derived from any species.

Standard techniques known to those of skill in the art can be used to introduce mutations in the nucleotide sequence encoding a cytokine, including, for example, site-directed mutagenesis and PCR-mediated mutagenesis which results in amino acid substitutions. Preferably, a derivative of a cytokine includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule.

In a preferred embodiment, a derivative of a cytokine has conservative amino acid substitutions made at one or more predicted non-essential amino acid residues (e.g., amino acid residues which are not critical for the cytokine to bind to its receptor). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded cytokine can be expressed and the activity of the cytokine can be determined by techniques well-known in the art or described herein.

Derivatives of cytokines also include cytokines modified, e.g., by the covalent attachment of any type of molecule to the cytokine. For example, but not by way of limitation, the derivatives of cytokines include cytokines that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention encompasses cytokines and fragments, derivatives and analogs thereof that immunospecifically bind to one or more subunits of a cytokine receptor fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the soluble LFA-3 polypeptide. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses cytokines and fragments, derivatives and analogs thereof that immunospecifically bind to one or more subunits of a cytokine receptor conjugated to a therapeutic agent. A cytokine and a fragment, derivative or analog thereof that immunospecifically binds to a cytokine receptor may be conjugated to a therapeutic moiety such as a cytotoxin, e.g, a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a cytokine and a fragment, derivative or analog thereof that immunospecifically binds to one or more subunits of a cytokine receptor may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as an apoptotic agent (see, International Publication No. WO 97/33899), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier.

5.2.1. Antibodies that Immunospecifically Bind to Cytokine Receptors

The present invention provides methods of preventing or treating cancer, an inflammatory disorder or an infectious disease by administering to a subject in need thereof one or more antibodies that immunospecifically bind to one or more subunits of a cytokine receptor and induce the activation of a signal transduction pathway associated with the cytokine receptor (i.e., agonistic antibodies that immunospecifically bind to a cytokine receptor) in combination with the administration of one or more co-stimulatory molecule-activating agents. The present invention provides methods of preventing or treating cancer, an inflammatory disorder or an infectious disease by administering to a subject in need thereof one or more nucleic acid molecules comprising nucleotide sequences encoding one or more agonistic antibodies that immunospecifically bind to one or more subunits of a cytokine receptor in combination with the administration of one or more co-stimulatory molecule-activating agents. The nucleotide sequence of agonistic antibodies that immunospecifically bind to one or more subunits of a cytokine receptor can be obtained, e.g., from the literature or a database such as GenBank. The present invention also provides compositions and kits comprising one or more agonistic antibodies that immunospecifically bind to one or more subunits of a cytokine receptor, or one or more nucleic acid molecules comprising nucleotide sequences encoding one or more agonistic antibodies that immunospecifically bind to one or more subunits of a cytokine receptor and one or more co-stimulatory molecule-activating agents.

It should be recognized that agonistic antibodies that immunospecifically bind to one or more subunits of a cytokine receptor are known in the art. Examples of agonistic antibodies that immunospecifically bind to one or more subunits of a cytokine receptor include, but are not limited to, antibodies that immunospecifically bind to and induce the activation of a signal transduction pathway associated with the IFN-α receptor, IFN-β receptor, IFN-γ receptor, TNF-α receptor, Flt3, IL-1β receptor, IL-2 receptor, IL-3 receptor, IL-4 receptor, IL-5 receptor, IL-6 receptor, IL-7 receptor, IL-8 receptor, IL-9 receptor, IL-10 receptor, IL-12 receptor, IL-15 receptor, IL-18 receptor, G-CSF receptor, M-CSF receptor, GM-CSF receptor, MIP-1 receptor, IP-10 receptor and MIG receptor. In accordance with the invention, commercially available antibodies, recombinant antibodies, or naturally occurring isolated antibodies may be used in the compositions, kits and invention.

In a specific embodiment, the agonistic antibody used in accordance with the invention is an agonistic antibody that immunospecifically binds to one or more subunits of a cytokine receptor and affects the biological activity of Th cells, NK cells and/or dendritic cells. In another embodiment, the agonistic antibody used in accordance with the invention is an agonistic antibody that immunospecifically binds to a cytokine receptor and promotes the differentiation of myeloid cells into dendritic cells and/or macrophages. In a preferred embodiment, the agonistic antibody used in accordance with the invention is an agonistic antibody that immunospecifically binds to one or more subunits of a cytokine receptor and promotes the differentiation of Gr-1⁺ myeloid progenitor cells into dendritic cells and/or macrophages. In another preferred embodiment, the agonistic antibody used in accordance with the invention is an agonistic antibody that immunospecifically binds to a cytokine receptor and promotes the differentiation of Gr-1⁺/CD11b⁺ myeloid progenitor cells into dendritic cells and/or macrophages. In a particular embodiment, the agonistic antibody used in accordance with the invention is an agonistic antibody that immunospecifically binds to the IL-12 receptor, IL-15 receptor, IL-18 receptor, Flt3, or GM-CSF receptor.

Agonistic antibodies that immunospecifically bind to one or more subunits of a cytokine receptor include, but are not limited to, monoclonal antibodies, multispecific antibodies, human antibodies, humanized antibodies, camelized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, agonistic antibodies that immunospecifically bind to one or more subunits of a cytokine receptor include immunoglobulin molecules and immunologically active portions of immunoglobulin molecules, i.e., molecules that contain an antigen binding site that immunospecifically binds to one or more subunits of a cytokine receptor. The immunoglobulin molecules of the invention can be of any type (e.g., IgG, IgE, IgM, IgD and IgA), class (e.g., IgG₁, IgG₂, IgG₃, IgG₄, IgA₁ and IgA₂) or subclass of immunoglobulin molecule. Preferably, the immunoglobulin molecule is an IgG molecule.

Agonistic antibodies that immunospecifically bind to one or more subunits of a cytokine receptor may be monospecific, bispecific, trispecific or of greater multispecificity. Multispecific antibodies may be specific for different epitopes of a cytokine receptor or may be specific for both a cytokine receptor as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for agonistic antibodies that have a high binding affinity for one or more subunits of a cytokine receptor. In a specific embodiment, an agonistic antibody that immunospecifically binds to one or more subunits of a cytokine receptor has an association rate constant or kₒₙ rate (antibody (Ab)+antigen

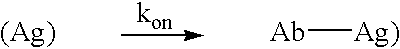

of at least 10⁵ M⁻¹ s⁻¹, at least 5×10⁵ M⁻¹ s⁻¹, least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In a preferred embodiment, an agonistic antibody that immunospecifically binds to one or more subunits of a cytokine receptor has a kₒₙ of at least 2×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹.

In another embodiment, an agonistic antibody that immunospecifically binds to one or more subunits of a cytokine receptor has a k_{off} rate (antibody (Ab)+antigen

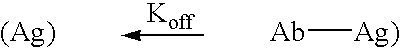

of less than 10⁻¹ s⁻¹, less than 5×10⁻³ s⁻¹, less than 10⁻² s⁻¹, less than 5×10⁻² s⁻¹, less than 10⁻¹ s⁻¹, less than 5×10⁻³ s⁻¹, less than 10⁻¹ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻¹ s⁻¹, less than 5×10⁻¹ s⁻¹, less than 10⁻⁹ s⁻¹, less than 5×10⁻⁹ s⁻¹, or less than 10⁻¹⁰ s⁻¹. In a preferred embodiment, an agonistic antibody that immunospecifically binds to one or more subunits of a cytokine receptor has a kₒₙ of less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In another embodiment, an agonistic antibody that immunospecifically binds to one or more subunits of a cytokine receptor has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10 M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an agonistic antibody that immunospecifically binds to one or more subunits of a cytokine receptor has a dissociation constant or K_{d} (k_{off}^{/k}ₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷M, less than 10⁻⁸ M, less than 5×10−8 M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

Agonistic antibodies that immunospecifically bind to one or more subunits of a cytokine receptor may be from any animal origin including birds and mammals (e.g., human, murine, camel, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins (e.g., the Xenomouse from Abgenix).

The invention provides for the use of functionally active fragments, derivatives or analogs of agonistic antibodies that immunospecifically bind to one or more subunits of a cytokine receptor. For example, a variable heavy (VH) domain, a VH complementarity determining region (CDR), a variable light (VL) domain, or a VL CDR of an agonistic antibody that immunopecifically binds to one or more subunits of a cytokine receptor can be used in accordance with the compositions and methods of the invention. In particular, a VH CDR3 or VL CDR3 of an agonistic antibody that immunospecifically binds to one or more subunits of a cytokine receptor can be used in accordance with the compositions and methods of the invention.

A derivative or analog of agonistic antibody that immunopecifically binds to one or more subunits of a cytokine receptor or antigen-binding region thereof (i.e., VH domain, a VH CDR, VL domain, or a VL CDR) can be used in accordance with the compositions and methods of the invention. Standard techniques known to those of skill in the art can be used to introduce mutations in the nucleotide sequence encoding an agonistic antibody that immunospecifically binds to a cytokine receptor, including, for example, site-directed mutagenesis and PCR-mediated mutagenesis which results in amino acid substitutions. Preferably, a derivative of an agonistic antibody that immunospecifically binds to one or more subunits of a cytokine receptor includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, a derivative of an agonistic antibody that immunospecifically binds to one or more subunits of a cytokine receptor has conservative amino acid substitutions made at one or more predicted non-essential amino acid residues (e.g., amino acid residues which are not critical for the antibody to immunospecifically bind to a cytokine receptor). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined by any technique well-known in the art or described herein. For example, the activity of the antibody can be determined by detecting the phosphorylation (i.e., tyrosine or serine/threonine) of the cytokine receptor or its substrate by immunoprecipitation followed by western blot analysis.

Derivatives of agonistic antibodies that immunospecifically bind to one or more subunits of a cytokine receptor also include antibodies modified, e.g., by the covalent attachment of any type of molecule to the antibodies. For example, but not by way of limitation, the derivatives of agonistic antibodies that immunospecifically bind to one or more subunits of a cytokine receptor include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The invention provides for the use of agonistic antibodies that immunospecifically bind to one or more subunits of a cytokine receptor comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody well-known in the art that immunospecifically binds to one or more subunits of a cytokine receptor. The invention also provides for the use of agonistic antibodies that immunospecifically bind to one or more subunits of a cytokine receptor encoded by nucleotide sequences that hybridize under stringent conditions to the nucleotide sequences encoding an antibody well-known in the art that immunospecifically binds to a cytokine receptor.

In a specific embodiment, an agonistic antibody that immunospecifically binds to one or more subunits of a cytokine receptor is a monoclonal antibody. In a preferred embodiment, an agonistic antibody that immunospecifically binds to one or more subunits of a cytokine receptor is a human or humanized monoclonal antibody. In another embodiment, the agonistic antibodies that immunospecifically bind to one or more subunits of a cytokine receptor comprise an Fe domain or a fragment thereof (e.g., the CH2, CH3, and/or hinge regions of an Fc domain).

The present invention also provides for the use of fusion proteins comprising an agonistic antibody that immunospecifically binds to one or more subunits of a cytokine receptor and a heterologous polypeptide. Preferably, the heterologous polypeptide that the antibody is fused to is useful for targeting the antibody to T-cells, NK cells and/or dendritic cells.

5.1.2.1. Agonistic Antibodies Having Increased Half-Lives That Immunospecifically Bind to Cytokine Receptors

The present invention provides for agonistic antibodies that immunospecifically bind to cytokine receptors and have an extended half-life in vivo. In particular, the present invention provides antibodies agonistic antibodies that immunospecifically bind to cytokine receptors and have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of agonistic antibodies that immunospecifically bind to cytokine receptors (e.g., monoclonal antibodies, single chain antibodies and Fab fragments) in vivo, inert polymer molecules such as high molecular weight polyethyleneglycol (PEG) can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized agonistic antibodies that immunospecifically bind to cytokine receptors can be tested for binding activity as well as for in vivo efficacy using methods known to those of skill in the art, for example, by immunoassays described herein.

Agonistic antibodies that immunospecifically bind to cytokine receptors and have an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

5.1.2.2. Antibody Conjugates

The present invention encompasses agonistic antibodies that immunospecifically bind to cytokine receptors recombinantly fused or chemically conjugated (including both covalently and non-covalently conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences.

The present invention also encompasses agonistic antibodies that immunospecifically bind to cytokine receptors fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses agonistic antibodies that immunospecifically bind to cytokine receptors conjugated to an agent which has a potential therapeutic benefit. An agonistic antibody that immunospecifically binds to one or more subunits of a cytokine receptor may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g, dactinomycin (formerly actinomycin), bleomycin, mithramycin, and anthramycin (AMC)), and anti-mitotic agents (e.g., vincristine and vinblastine).

Further, an agonistic antibody that immunospecifically binds to one or more subunits of a cytokine receptor may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as an apoptotic agent (see, International Publication No. WO 97/33899), AIM II (see, International Publication No. WO 97/34911), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine or a growth factor (e.g., growth hormone (“GH”)).

Techniques for conjugating such therapeutic moieties to antibodies are well known, see, e.g., Arnon et a., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and Thorpe et al., 1982, Immunol. Rev. 62:119-58.

An agonistic antibody that immunospecifically binds to one or more subunits of a cytokine receptor can be conjugated to a second antibody to form an antibody heteroconjugate as described by Segal in U.S. Pat. No. 4,676,980, which is incorporated herein by reference in its entirety.

5.2. Co-Stimulatory Molecule-Activating Agents

Any compound well-known to one of skill in the art that immunospecifically binds to or associates with a co-stimulatory molecule expressed by an immune cell (preferably, an activated immune cell) and induces the activation of a signal transduction pathway associated the co-stimulatory molecule (i.e., a cytokine receptor-activating agent) may be used in the methods and compositions of the invention. Preferably, a compound well-known to one of skill in the art that immunospecifically binds to or associates with a co-stimulatory molecule selectively expressed by an activated immune cell (preferably, an activated T-cell) and induces the activation of a signal transduction pathway associated with the co-stimulatory molecule is used in the methods and compositions of the invention. Co-stimulatory molecule-activating agents include, but are not limited to, proteinaneous agents (e.g., cytokines, peptide mimetics, and antibodies), small molecules, organic compounds, inorganic compounds, and nucleic acid molecules comprising nucleotide sequences encoding proteins, polypeptides, or peptides (e.g., cytokines, peptide mimetics, and antibodies) that immunospecifically bind to or associate with a co-stimulatory molecule expressed by an activated immune cell and induce the activation of a signal transduction pathway associated with the co-stimulatory molecule.

In certain embodiments, the co-stimulatory molecule-activating agent is a protein, polypeptide, or peptide (i.e., a co-stimulatory molecule-activating polypeptide) that immunospecifically binds to or associates with a co-stimulatory molecule expressed by an activated immune cell and induces the activation of a signal transduction pathway associated with the co-stimulatory molecule. In other embodiments, the co-stimulatory molecule-activating agent is a nucleic acid molecule comprising a nucleotide sequence encoding a protein, polypeptide or peptide that immunospecifically binds to or associate with a co-stimulatory molecule expressed by an activated immune cell and induces the activation of a signal transduction pathway associated with the co-stimulatory molecule. In certain other embodiments, the co-stimulatory molecule-activating agent is a fusion protein or a nucleic acid molecule comprising a nucleotide sequence encoding a fusion protein, said fusion protein comprising a protein, polypeptide, or peptide that immunospecifically binds to or associates with a co-stimulatory molecule expressed by an activated immune cell and induces the activation of a signal transduction pathway associated with the co-stimulatory molecule fused to a heterologous protein, polypeptide or peptide. In yet other embodiments, the co-stimulatory molecule-activating agent is not fusion protein or a nucleic acid molecule comprising a nucleotide sequence encoding a fusion protein.

In a preferred embodiment, the co-stimulatory molecule-activating agent is a native or recombinant protein polypeptide, peptide, fragment, derivative or analog thereof that immunospecifically binds to a co-stimulatory molecule expressed by activated immune cells (preferably, activated T-cells), preferably a co-stimulatory molecule selectively expressed by activated immune cells (preferably, activated T-cells), and activates a signal transduction pathway associated with the co-stimulatory molecule. In another preferred embodiment, the co-stimulatory molecule-activating agent is a nucleic acid molecule comprising a nucleotide sequence encoding a protein, polypeptide, or peptide that immunospecifically binds to a co-stimulatory molecule expressed by activated immune cells (preferably, activated T-cells), preferably a co-stimulatory molecule selectively expressed by activated immune cells (preferably, activated T-cells), and activates a signal transduction pathway associated with the co-stimulatory molecule. In another embodiment, the co-stimulatory molecule-activating agent is a ligand for a co-stimulatory molecule (such as, e.g., SLAM, OX40, 4-1BB, inducible co-stimulator (ICOS), B7RP-1 and CD27) expressed by activated T-cells, with the proviso that the ligand is not B7-1. Examples of such ligands, include, but are not limited to, 4-1BBL, SLAM, CD40 ligand (CD40L),CD70 ligand (CD70L) and OX-40L. In another embodiment, the co-stimulatory molecule-activating agent is expressed by dendritic cells (e.g., CD40).

5.2.1. Ligands That Immunospecifically Bind to Co-Stimulatory Molecules

The present invention encompasses compositions, kits, and methods utilizing one or more ligands immunospecific for one or more co-stimulatory molecules expressed by immune cells (preferably, activated immune cells). The present invention also encompasses compositions, kits, and methods utilizing one or more nucleic acid molecules comprising nucleotide sequences encoding one or more ligands immunospecific for one or more co-stimulatory molecules expressed by immune cells (preferably, activated immune cells). The present invention also encompasses compositions, kits, and methods utilizing one or more ligands immunospecific for one or more co-stimulatory molecules selectively expressed by activated immune cells. The present invention further encompasses compositions, kits, and methods utilizing one or more nucleic acid molecules encoding one or more ligands immunospecific for one or more co-stimulatory molecules selectively expressed by activated immune cells. Preferably, the ligands utilized in accordance with the invention immunospecifically bind to co-stimulatory molecules selectively expressed by activated T-cells. The nucleotide sequences and/or amino acid sequences of ligands immunospecific for co-stimulatory molecules expressed by activated immune cells can be obtained, e.g., from the literature or a database such as GenBank. -For example, the nucleotide sequences of 4-1BBL and OX40L can be found in GenBank under GenBank Accession Nos. U03398 and X79929, respectively.

In a specific embodiment, ligands that immunospecifically bind to a co-stimulatory molecule selectively expressed by activated T-cells augment the activation of activated T-cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In another embodiment, ligands that immunospecifically bind to a co-stimulatory molecule selectively expressed by activated T-cells increase the proliferation of activated T-cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, ligands that immunospecifically bind to a co-stimulatory molecule selectively expressed by activated T-cells increase the expression and/or release of cytokines by immune cells in an in vitro or in vivo assay described herein or known to one of skill in the art. In a specific embodiment, ligands that immunospecifically bind to a co-stimulatory molecule selectively expressed by activated T-cells increase the concentration of cytokines such as, e.g., IFN-α, IFN-β, IFN-γ, IL-2, IL-3, IL-4, IL-6, IL-7; IL-9, IL-10, IL-12, IL-15, IL-18 and TNF-α in the serum of a subject administered such ligands. Serum concentrations of a cytokine can be measured by any technique known to one of skill in the art such as, e.g., ELISA.

The present invention encompasses compositions, kits and methods utilizing fragments, derivatives and analogs of ligands that immunospecifically bind to a co-stimulatory molecule selectively expressed by immune cells (preferably, activated immune cells such as activated T-cells). Preferably, fragments, derivatives and analogs of ligands that immunospecifically bind to a co-stimulatory molecule selectively expressed by activated immune cells retain the ability to immunospecifically bind to a co-stimulatory molecule selectively expressed by activated immune cells and induce the activation of a signal transduction pathway associated with the co-stimulatory molecule. Ligands and fragments, derivatives and analogs thereof that immunospecifically bind to a co-stimulatory molecule can be derived from any species.

Standard techniques known to those of skill in the art can be used to introduce mutations in the nucleotide sequence encoding a ligand that immunospecifically binds to a co-stimulatory molecule, including, for example, site-directed mutagenesis and PCR-mediated mutagenesis which results in amino acid substitutions. Preferably, a derivative of ligand that immunospecifically binds to a co-stimulatory molecule includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, a derivative of a ligand that immunospecifically binds to a co-stimulatory molecule has conservative amino acid substitutions made at one or more predicted non-essential amino acid residues (e.g., amino acid residues which are not critical for the cytokine to bind to its receptor). Alternatively, mutations can be introduced randomly along all or part of the coding sequence, such as by saturation mutagenesis, and the resultant mutants can be screened for biological activity to identify mutants that retain activity. Following mutagenesis, the encoded ligand can be expressed and the activity of the ligand can be determined by techniques well-known in the art or described herein.

Derivatives of ligands that immunospecifically bind to a co-stimulatory molecule also include ligands modified, e.g., by the covalent attachment of any type of molecule to the cytokine. For example, but not by way of limitation, the derivatives of ligands that immunospecifically bind to a co-stimulatory molecule include ligands that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention encompasses ligands and fragments, derivatives and analogs thereof that immunospecifically bind to a co-stimulatory molecule fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the soluble LFA-3 polypeptide. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses ligands and fragments, derivatives and analogs thereof that immunospecifically bind to a co-stimulatory molecule conjugated to a therapeutic agent. A ligand and a fragment, derivative or analog thereof that immunospecifically binds to a co-stimulatory molecule may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a ligand and a fragment, derivative or analog thereof that immunospecifically binds to a co-stimulatory may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as an apoptotic agent (see, International Publication No. WO 97/33899), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as a lymphokine or growth factor.

5.2.2. Antibodies That Immunospecifically Bind to Co-Stimulatory Molecules

The present invention encompasses compositions, kits and methods utilizing one or more agonistic antibodies that immunospecifically bind to one or more co-stimulatory molecules expressed by immune cells (preferably, activated immune cells). The present invention also encompasses compositions, kits and methods utilizing one or more nucleic acid molecules comprising nucleotide sequences encoding one or more agonistic antibodies that immunospecifically bind to one or more co-stimulatory molecules expressed by immune cells (preferably, activated immune cells). The present invention also encompasses the use of one or more agonistic antibodies that immunospecifically bind to one or more co-stimulatory molecules selectively expressed by activated immune cells. The present invention further encompasses compositions, kits and methods utilizing one or more nucleic acid molecules comprising nucleotide sequences encoding one or more agonistic antibodies that immunospecifically bind to one or more co-stimulatory molecules selectively expressed by activated immune cells. Preferably, the agonistic antibodies utilized in accordance with the invention immunospecifically bind to co-stimulatory molecules selectively expressed by activated T-cells. The nucleotide sequence of agonistic antibodies that immunospecific for co-stimulatory molecules expressed by activated immune cells can be obtained, e.g., from the literature or a database such as GenBank.

In a specific embodiment, an agonistic antibody that immunospecifically binds to a co-stimulatory molecule selectively expressed by activated T-cells augments the activation of the activated T-cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In another embodiment, an agonistic antibody that immunospecifically binds to a co-stimulatory molecule selectively expressed by activated T-cells increases the proliferation of the activated T-cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, an agonistic antibody that immunospecifically binds to a co-stimulatory molecule selectively expressed by activated T-cells increases the expression and/or release of cytokines by immune cells in an in vitro or in vivo assay described herein or known to one of skill in the art. In a specific embodiment, an agonistic antibody that immunospecifically binds to a co-stimulatory molecule selectively expressed by activated T-cells increases the concentration of cytokines such as, e.g., IFN-α, IFN-β, IFN-γ, IL-2, IL-4, IL-6, IL-7, IL-9, IL-10, IL-12, IL-15, IL-18 and TNF-α in the serum of a subject administered such ligands. Serum concentrations of a cytokine can be measured by any technique known to one of skill in the art such as, e.g., ELISA.

It should be recognized that agonistic antibodies that immunospecifically bind to a co-stimulatory molecule are known in the art. Examples of agonistic antibodies that immunospecifically bind to a co-stimulatory molecule include, but are not limited to, antibodies that immunospecifically bind to and induce the activation of a signal transduction pathway associated with 4-1BB, OX40, CD40, SLAM, ICOS, B7RP-1 and CD27. In accordance with the invention, commercially available antibodies, recombinant antibodies, or naturally occurring isolated antibodies may be used in the compositions, kits and invention.

Agonistic antibodies that immunospecifically bind to a co-stimulatory molecule include, but are not limited to, monoclonal antibodies, multispecific antibodies, human antibodies, humanized antibodies, camelized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, agonistic antibodies that immunospecifically bind to a co-stimulatory molecule include immunoglobulin molecules and immunologically active portions of immunoglobulin molecules, i.e., molecules that contain an antigen binding site that immunospecifically binds to a co-stimulatory molecule. The immunoglobulin molecules of the invention can be of any type (e.g., IgG, IgE, IgM, IgD, and IgA), class (e.g, IgG₁, IgG₂, IgG₃, IgG₄, IgA₁ and IgA₂) or subclass of immunoglobulin molecule. Preferably, the immunoglobulin molecule is an IgG molecule.

Agonistic antibodies that immunospecifically bind to a co-stimulatory molecule may be monospecific, bispecific, trispecific or of greater multispecificity. Multispecific antibodies may be specific for different epitopes of a co-stimulatory molecule or may be specific for both a co-stimulatory molecule as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for agonistic antibodies that have a high binding affinity for a co-stimulatory molecule. In a specific embodiment, an agonistic antibody that immunospecifically binds to a co-stimulatory molecule has an association rate constant or kₒₙ rate (antibody (Ab)+antigen

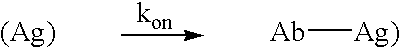

of at least 10⁵ M⁻¹ s⁻¹, at least 5×10⁵ M⁻¹ s⁻¹, least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In a preferred embodiment, an agonistic antibody that immunospecifically binds to a co-stimulatory molecule has a kₒₙ of at least 2×10⁵ M⁻¹ s⁻¹, at least 5×10⁵ M⁻¹ s⁻¹, at least 10⁶ M⁻¹ s⁻¹ at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹.

In another embodiment, an agonistic antibody that immunospecifically binds to a co-stimulatory molecule has a k_{off} rate (antibody (Ab)+antigen

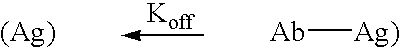

of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10-6 s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a preferred embodiment, an agonistic antibody that immunospecifically binds to a co-stimulatory molecule has a kₒₙ of less than 5×10⁻⁴ s⁻¹, less than 10⁻⁵ s^{31 1}, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In another embodiment, an agonistic antibody that immunospecifically binds to a co-stimulatory molecule has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an agonistic antibody that immunospecifically binds to a co-stimulatory molecule has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³M, less than 5×10⁻¹³ M, less than 10⁻¹⁴ M, less than 5×10⁻¹⁴ M, less than 10⁻¹⁵ M, or less than 5×10⁻¹⁵ M.

Agonistic antibodies that immunospecifically bind to a co-stimulatory molecule may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins (e.g., the Xenomouse from Abgenix).

The invention provides for the use of functionally active fragments, derivatives or analogs of agonistic antibodies that immunospecifically bind to a co-stimulatory molecule. For example, a variable heavy (VH) domain, a VH complementarity determining region (CDR), a variable light (VL) domain, or a VL CDR of an agonistic antibody that immunopecifically binds to a co-stimulatory molecule can be used in accordance with the compositions and methods of the invention. In particular, a VH CDR3 or VL CDR3 of an agonistic antibody that immunospecifically binds to a co-stimulatory molecule can be used in accordance with the compositions and methods of the invention.

A derivative or analog of agonistic antibody that immunospecifically binds to a co-stimulatory molecule or antigen-binding region thereof (i.e., VH domain, a VH CDR, VL domain, or a VL CDR) can be used in accordance with the compositions and methods of the invention. Standard techniques known to those of skill in the art can be used to introduce mutations in the nucleotide sequence encoding an agonistic antibody that immunospecifically binds to a co-stimulatory molecule, including, for example, site-directed mutagenesis and PCR-mediated mutagenesis which results in amino acid substitutions. Preferably, a derivative of an agonistic antibody that immunospecifically binds to a co-stimulatory molecule includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, a derivative of an agonistic antibody that immunospecifically binds to a co-stimulatory molecule has conservative amino acid substitutions made at one or more predicted non-essential amino acid residues (e.g., amino acid residues which are not critical for the antibody to immunospecifically bind to a cytokine receptor). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined by any technique well-known in the art or described herein. For example, the activity of the antibody can be determined by detecting the phosphorylation (i.e., tyrosine or serine/threonine) of the co-stimulatory molecule or its substrate by immunoprecipitation followed by western blot analysis.

Derivatives of agonistic antibodies that immunospecifically bind to a co-stimulatory molecule also include antibodies modified, e.g., by the covalent attachment of any type of molecule to the antibodies. For example, but not by way of limitation, the derivatives of agonistic antibodies that immunospecifically bind to a co-stimulatory molecule include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The invention provides for the use of agonistic antibodies that immunospecifically bind to a co-stimulatory molecule comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody well-known in the art that immunospecifically binds to a co-stimulatory molecule. The invention also provides for the use of agonistic antibodies that immunospecifically bind to a co-stimulatory molecule encoded by nucleotide sequences that hybridize under stringent conditions to the nucleotide sequences encoding an antibody well-known in the art that immunospecifically binds to a co-stimulatory molecule.

In a specific embodiment, an agonistic antibody that immunospecifically binds to a co-stimulatory molecule is a monoclonal antibody. In a preferred embodiment, an agonistic antibody that immunospecifically binds to a co-stimulatory molecule is a human or humanized monoclonal antibody. In another embodiment, the agonistic antibodies that immunospecifically bind to a co-stimulatory molecule comprise an Fe domain or a fragment thereof (e.g., the CH2, CH3, and/or hinge regions of an Fe domain).

The present invention also provides for the use fusion proteins comprising an agonistic antibody that immunospecifically binds to a co-stimulatory molecule and a heterologous polypeptide.

5.2.2.1. Agonistic Antibodies Having Increased Half-Lives That Immunospecifically Bind to Co-Stimulatory Molecules

The present invention provides for agonistic antibodies that immunospecifically bind to co-stimulatory molecules and have an extended half-life in vivo. In particular, the present invention provides antibodies agonistic antibodies that immunospecifically bind to co-stimulatory molecules and have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of agonistic antibodies that immunospecifically bind to co-stimulatory molecules (e.g., monoclonal antibodies, single chain antibodies and Fab fragments) in vivo, inert polymer molecules such as high molecular weight polyethyleneglycol (PEG) can be attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized agonistic antibodies that immunospecifically bind to cytokine receptors can be tested for binding activity as well as for in vivo efficacy using methods known to those of skill in the art, for example, by immunoassays described herein. Agonistic antibodies that immunospecifically bind to co-stimulatory molecules and have an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

5.2.2.2. Antibody Conjugates

The present invention encompasses agonistic antibodies that immunospecifically bind to co-stimulatory molecules recombinantly fused or chemically conjugated (including both covalently and non-covalently conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences.

The present invention also encompasses agonistic antibodies that immunospecifically bind to co-stimulatory molecules fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses agonistic antibodies that immunospecifically bind to co-stimulatory molecules conjugated to an agent which has a potential therapeutic benefit. An agonistic antibody that immunospecifically binds to a co-stimulatory molecule may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an agonistic antibody that immunospecifically binds to a co-stimulatory molecule may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as an apoptotic agent (see, International Publication No. WO 97/33899), AIM II (see, International Publication No. WO 97/34911), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection nd Therapy, Baldwin et al. (eds.), pp. 303-16 (Academic Press 1985); and Thorpe et al., 1982, Immunol. Rev. 62:119-58.

An agonistic antibody that immunospecifically binds to a co-stimulatory molecule an be conjugated to a second antibody to form an antibody heteroconjugate as described by Segal in U.S. Pat. No. 4,676,980, which is incorporated herein by reference in its entirety.

5.3. Expression of Nucleic Acid Molecules Encoding Cytokine Receptor-Activating Polypeptides and/or Co-Stimulatory Molecule-Activating Polypeptides

The nucleotide sequence encoding a cytokine receptor-activating polypeptide can be inserted into an appropriate expression vector, i.e., a vector which contains the necessary elements for the transcription and translation of the inserted protein-coding sequence. In a specific embodiment, the nucleotide sequence encoding a cytokine (e.g., IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, G-CSF, GM-CSF, M-CSF and chemokines) or a functionally active analogs or fragments or other derivatives thereof is inserted into an appropriate expression vector. The nucleotide sequence encoding a co-stimulatory molecule-activating polypeptide can be inserted into an appropriate expression vector, i.e., a vector which contains the necessary elements for the transcription and translation of the inserted protein-coding sequence. In a specific embodiment, the nucleotide sequence encoding a ligand immunospecific for a co-stimulatory molecule expressed on activated T-cells (e.g., 4-1BBL, CD40, SLAM, CD70 ligand (CD70L) and OX-40L) or a functionally active analogs or fragments or other derivatives thereof is inserted into an appropriate expression vector.

The necessary transcriptional and translational signals can also be supplied by the native cytokine receptor-activating polypeptide or native co-stimulatory molecule-activating polypeptide genes or its flanking regions. A variety of host-vector systems may be utilized to express the protein-coding sequence. These include but are not limited to mammalian cell systems infected with virus (e.g., vaccinia virus, adenovirus, adeno-associated virus (AAV), retrovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In specific embodiments, nucleotide sequences encoding human IL-12 and human 4-1BB ligand are expressed in vivo, or nucleotide sequences encoding functionally active fragments, derivatives or analogs of human IL-12 and human 4-1BB ligand are expressed in vivo. In another embodiment, nucleotide sequences encoding human IL-12 and human OX-40 ligand are expressed in vivo, or nucleotide sequences encoding functionally active fragments, derivatives or analogs of human IL-12 and human OX-40 ligand are expressed in vivo. In another embodiment, nucleotide sequences encoding human IL-12, human 4-1BB ligand, and human OX40 ligand are expressed in vivo, or nucleotide sequences encoding functionally active fragments, derivatives or analogs of human IL-12, human 4-1BB ligand, and human OX40 ligand are expressed in vivo. In another embodiment, nucleotide sequences encoding human IL-12, human 4-1BB ligand, and human GM-CSF are expressed in vivo, or nucleotide sequences encoding functionally active fragments, derivatives or analogs of human IL-12, human 4-1BB ligand, and GM-CSF are expressed in vivo. In another embodiment, nucleotide sequences encoding human IL-12, human OX40 ligand, and human GM-CSF are expressed in vivo, or nucleotide sequences encoding functionally active fragments, derivatives or analogs of human IL-12, human OX40 ligand, and GM-CSF are expressed in vivo. In yet another embodiment, nucleotide sequences encoding human IL-12, human 4-1BB ligand, human OX40 ligand, and human GM-CSF are expressed in vivo, or nucleotide sequences encoding functionally active fragments, derivatives or analogs of human IL-12, human 4-1BB ligand, human OX40 ligand, and GM-CSF are expressed in vivo.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of the nucleic acid sequence encoding a cytokine receptor-activating polypeptide or a co-stimulatory molecule-activating polypeptide may be regulated by a second nucleic acid sequence so that the cytokine receptor-activating polypeptide or co-stimulatory molecule-activating polypeptide are expressed in a host transformed with the recombinant DNA molecule. For example, expression of IL-12, 4-1BB ligand, OX40 ligand, or GM-CSF may be controlled by any promoter or enhancer element known in the art. Constitutively active promoter elements, inducible promoter elements or tissue-specific promoter elements may be used to express a cytokine receptor-activating polypeptide or a co-stimulatory molecule-activating polypeptide.

Promoters which may be used to control the expression of a cytokine receptor-activating polypeptide and/or a co-stimulatory molecule-activating polypeptide include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. USA 75:3727-373 1), or the tac promoter (DeBoer et al., 1983, Proc. Natl. Acad. Sci. USA 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlaufet al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells, (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

In a specific embodiment, a vector used in accordance with the invention comprises a promoter operably linked to a cytokine receptor-activating polypeptide-encoding nucleic acid, one or more origins of replication, and, optionally, one or more selectable markers (e.g., an antibiotic resistance gene). In another embodiment, a vector used in accordance with the invention comprises a promoter operably linked to a co-stimulatory molecule-activating polypeptide-encoding nucleic acid, one or more origins of replication, and, optionally, one or more selectable markers (e.g., an antibiotic resistance gene). In yet another embodiment, a vector used in accordance with the invention comprises a promoter operably linked to a cytokine receptor-activating polypeptide and co-stimulatory molecule-activating polypeptide-encoding nucleic acids, one or more origins of replication, and, optionally, one or more selectable markers (e.g., an antibiotic resistance gene).

Expression vectors containing gene inserts can be identified by three general approaches: (a) nucleic acid hybridization; (b) presence or absence of “marker” gene functions; and (c) expression of inserted sequences. In the first approach, the presence of a cytokine receptor-activating polypeptide gene or a co-stimulatory molecule-activating polypeptide gene inserted in an expression vector(s) can be detected by nucleic acid hybridization using probes comprising sequences that are homologous to the inserted gene(s). In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of the gene(s) in the vector(s). For example, if the IL-12 gene is inserted within the marker gene sequence of the vector, recombinants containing the IL-12 gene insert can be identified by the absence of the marker gene function. In the third approach, recombinant expression vectors can be identified by assaying the gene product expressed by the recombinant. Such assays can be based, for example, on the physical or functional properties of the cytokine receptor-activating polypeptide and/or co-stimulatory molecule-activating polypeptide in in vitro assay systems, e.g., binding of IL-12 with anti-IL-12 antibody or binding of 4-1BB ligand with anti-4-1BB antibody.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Both cDNA and genomic sequences can be cloned and expressed.

5.4. Methods of Producing Antibodies

The antibodies that immunospecifically bind to an antigen (e.g., a cytokine receptor or co-stimulatory molecule) can be produced by any method known in the art for the synthesis of antibodies, in particular, by chemical synthesis or preferably, by recombinant expression techniques.

Polyclonal antibodies immunospecific for an antigen can be produced by various procedures well known in the art. For example, a human antigen (e.g., a human cytokine receptor or human co-stimulatory molecule) can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen (e.g., a non-murine cytokine receptor or a non-murine co-stimulatory molecule) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen (e.g., cytokine receptor or co-stimulatory molecule) with myeloma cells and then screening the hybridomas resulting from the fusion for hybridoma clones that secrete an antibody able to bind to the antigen. Antibody fragments which recognize specific particular epitopes (e.g., cytokine receptor epitopes or co-stimulatory molecule epitopes) may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine CDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen (e.g., a cytokine receptor or co-stimulatory molecule) can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g, as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a human antibody and a non-human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332). In a preferred embodiment, chimeric antibodies comprise a human CDR3 having an amino acid sequence of any one of the CDR3 listed in Table 1 and non-human framework regions. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.) Further, the antibodies that immunospecifically bind to an antigen (e.g., a cytokine receptor or co-stimulatory molecule) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

5.4.1. Recombinant Expression of Antibodies

The invention provides nucleotide sequences encoding an antibody or fragment thereof that immunospecifically binds to an antigen (e.g., cytokine receptor or co-stimulatory molecule). Nucleotide sequences encoding an antibody may be obtained or determined by any method known in the art. The nucleotide sequences of antibodies immunospecific for antigen can be obtained, e.g., from the literature or a database such as GenBank.

Recombinant expression of an antibody that immunospecifically binds to an antigen (e.g., a cytokine receptor or co-stimulatory molecule) requires construction of an expression vector containing a nucleotide sequence that encode the antibody. Once a nucleotide sequence encoding an antibody molecule of the invention has been obtained, the vector for the production of the antibody molecule may be produced by recombinant DNA technology using techniques well known in the art. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a nucleotide sequence encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to one or more antigens is regulated by a constitutive promoter, inducible promoter or tissue-specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcriptionltranslation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, W138, BT483, Hs578T, HTB2, BT20, T47D, NSO (a murine myeloma cell line that does not endogenously produce any immunoglobulin chains), CRL7030 and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule. A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt- cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.5. Prophylactic & Therapeutic Uses of Combination Therapy

The present invention is directed to combination therapies for the prevention or treatment of diseases and disorders, including cancer, inflammatory diseases and infectious diseases. In a preferred embodiment, one or more cytokine receptor-activating agents and one or more co-stimulatory molecule-activating agents are administered to a subject to prevent or treat cancer. Examples of types of cancer, include, but are not limited to, leukemia (e.g., acute leukemia such as acute lymphocytic leukemia and acute myelcytic leukemia), neoplasms, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease, metastases, or any disease or disorder characterized by uncontrolled cell growth. In certain embodiments, the combination therapies of the invention are not administered to subjects with cancer associated with immune cells such as, e.g., T-cell malignancies.

In a specific embodiment, one or more cytokine receptor-activating agents and one or more co-stimulatory molecule-activating agents are administered to a subject to prevent or treat an inflammatory disorder. Examples of inflammatory disorders include, but are not limited to, systemic lupus erythematosus, rheumatoid arthritis, acute respiratory distress syndrome, asthma, and osteoporosis).

In another embodiment, one or more cytokine receptor-activating agents and one or more co-stimulatory molecule-activating agents are administered to a subject to prevent or treat an infectious disease. Infectious diseases include, but are not limited, diseases associated with yeast, fungal, viral and bacterial infections. Viruses causing viral infections include, but are limited to, herpes simplex virus (HSV), hepatitis B virus (HBV), hepatitis C virus (HCV), human T-cell lymphotrophic virus (HTLV) type I and II, human immunodeficiency virus (HIV) type I and II, cytomegalovirus, papillomavirus, polyoma viruses, adenoviruses, Epstein-Barr virus, poxviruses, influenza virus, measles virus, rabies virus, Sendai virus, poliomyelitis virus, coxsackieviruses, rhinoviruses, reoviruses, and rubella virus. Microbial pathogens causing bacterial infections include, but are not limited to, Streptococcus pyogenes, Streptococcus pneumoniae, Neisseria gonorrhoea, Neisseria meningitidis, Corynebacterium diphtheriae , Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacterjejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

5.6. Therapeutic/Prophylactic Administration and Compositions

The present invention provides compositions and methods for the prevention and treatment of cancer, an inflammatory disorder, and an infectious disease. In particular, the invention provides therapeutic and pharmaceutical compositions comprising pharmaceutically acceptable carriers, one or more cytokine receptor-activating agents, and one or more co-stimulatory molecule-activating agents. The pharmaceutical compositions of the invention may be used in accordance with the methods of the invention for the treatment of cancer, an inflammatory disorder, or an infectious disease in a subject. The pharmaceutical compositions of the present invention are in suitable formulation to be administered to animals, preferably mammals such as companion animals (e.g., dogs, cats, and horses) and livestock (e.g., cows and pigs), and most preferably humans.

The present invention provides therapeutic or pharmaceutical compositions comprising a pharmaceutical carrier, one or more cytokine receptor-activating agents which affect the biological activity (e.g., differentiation, proliferation or effector function) of T helper (Th) cells and/or NK cells, and one or more co-stimulatory molecule-activating agents. In a specific embodiment, a pharmaceutical composition comprises a pharmaceutical carrier, one or more compounds that activate the IL-15 receptor, and one or more co-stimulatory molecule-activating agents. In another embodiment, a pharmaceutical composition comprises a pharmaceutical carrier, one or more compounds that activate the IL-18 receptor, and one or more co-stimulatory molecule-activating agents. In another embodiment, a pharmaceutical composition comprises a pharmaceutical carrier, one or more compounds that activate Flt3, and one or more co-stimulatory molecule-activating agents. The invention provides therapeutic and pharmaceutical compositions comprising pharmaceutically acceptable carriers, one or more compounds that activate the IL-12 receptor, and one or more co-stimulatory molecule-activating agents. In one embodiment, a pharmaceutical composition comprises a pharmaceutically acceptable carrier, one or more compounds that activate the IL-12 receptor, and one or more compounds that activate 4-1BB. In another embodiment, a pharmaceutical composition comprises a pharmaceutical carrier, a recombinant adenovirus expressing IL-12, and an agonistic anti-4-1BB antibody or an antigen-binding fragment thereof. In another embodiment, a pharmaceutical composition comprises a pharmaceutically acceptable carrier, one or more compounds that activate the IL-12 receptor, and an effective amount of one or more compounds that activate OX40. In another embodiment, a pharmaceutical composition comprises a pharmaceutical carrier, a recombinant adenovirus expressing IL-12, and an agonistic anti-OX40 monoclonal antibody or antigen-binding fragment thereof. In a preferred embodiment, a pharmaceutical composition comprises a pharmaceutically acceptable carrier, one or more compounds that activate the IL-12 receptor, one or more compounds that activate 4-1BB, and one or more compounds that activate OX40. In another preferred embodiment, a pharmaceutical composition comprises a pharmaceutical carrier, a recombinant adenovirus expressing IL-12, an agonistic anti-4-1BB monoclonal antibody or antigen-binding fragment thereof, and an agonistic anti-OX40 monoclonal antibody or antigen-binding fragment thereof.

In another embodiment, a pharmaceutical composition comprises a pharmaceutically acceptable carrier, one or more compounds that activate the IL-12 receptor, one or more compounds that activate 4-1BB, and one or more compounds that activate SLAM, ICOS, B7RP-1 or CD27. In another embodiment, a pharmaceutical composition comprises a pharmaceutically acceptable carrier, one or more compounds that activate the IL-12 receptor, one or more compounds that activate OX40, and one or more compounds that activate SLAM, ICOS, B7RP-1 or CD27. In yet another embodiment, a pharmaceutical composition comprises a pharmaceutically acceptable carrier, one or more compounds that activate the IL-12 receptor, one or more compounds that activate 4-1BB, one or more compounds that activate OX40, and one or more compounds that activate SLAM, ICOS, B7RP-1 or CD27.

The invention provides therapeutic and pharmaceutical compositions comprising pharmaceutically acceptable carriers, one or more compounds that activate the IL-12 receptor, one or more compounds that activate at least one cytokine receptor other than the IL-12 receptor, and one or more co-stimulatory molecule-activating agents. In one embodiment, a pharmaceutical composition comprises a pharmaceutical carrier, one or more compounds that activate the IL-12 receptor, one or more compounds that activate at least one cytokine receptor other the IL-12 receptor (e.g., one or more cytokines such as IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, GM-CSF, G-CSF, CSF-1, and M-CSF), and one or more co-stimulatory molecule-activating agents. In another embodiment, a pharmaceutical composition comprises a pharmaceutical carrier, one or more compounds that activate the IL-12 receptor, one or more compounds that activate the IL-15 receptor, and one or more co-stimulatory molecule-activating agents. In another embodiment, a pharmaceutical composition comprises a pharmaceutical carrier, one or more compounds that activate the IL-12 receptor, one or more compounds that activate the IL-18 receptor, and one or more co-stimulatory molecule-activating agents.

The present invention provides therapeutic or pharmaceutical compositions comprising a pharmaceutical carrier, one or more cytokine receptor-activating agents which affect the biological activity (e.g., differentiation, proliferation or effector function) of T helper (Th) cells and/or NK cells, and one or more co-stimulatory molecule-activating agents which affect the biological activity (e.g., differentiation, proliferation or effector function) of dendritic cells and/or macrophages. In a specific embodiment, the present invention provides a pharmaceutical composition comprising a pharmaceutical carrier, one or more compounds that activate the GM-CSF receptor and one or more compounds that activate CD40. In another embodiment, the present invention provides a pharmaceutical composition comprising a pharmaceutical carrier, one or more compounds that activate the GM-CSF receptor, and one or more compounds that activate 4-1BB.

The present invention provides therapeutic or pharmaceutical compositions comprising a pharmaceutical carrier, one or more cytokine receptor-activating agents which affect the biological activity (e.g., differentiation, proliferation or effector function) of T helper (Th) cells and/or NK cells, one or more cytokine receptor-activating agents which promote the differentiation of myeloid cells into dendritic cells and/or macrophages, and one or more co-stimulatory molecule-activating agents which affect the biological activity (e.g., differentiation, proliferation or effector function) of dendritic cells and/or macrophages. In one embodiment, the present invention provides the present invention provides a pharmaceutical composition comprising a pharmaceutical carrier, one or more compounds that activate the IL-12 receptor, one or more compounds that activate the GM-CSF receptor, and one or more compounds that activate CD40.

The present invention provides therapeutic or pharmaceutical compositions comprising a pharmaceutical carrier, one or more co-stimulatory molecule-activating agents, an effective amount of one or more cytokine receptor-activating agents which affect the biological activity (e.g., differentiation, proliferation or effector function) of T helper (Th) cells and/or NK cells, and one or more cytokine receptor-activating agents which promote the differentiation of myeloid cells into dendritic cells and/or macrophages. In a preferred embodiment, a pharmaceutical composition comprises a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of Gr-1⁺ myeloid progenitor cells into dendritic cells and/or macrophages. In another preferred embodiment, a pharmaceutical composition comprises a pharmaceutical carrier, one or more co-stimulatory molecule-activating agents, one or more cytokine receptor-activating agents which affect the biological activity (e.g., differentiation, proliferation or effector function) of T helper (Th) cells and/or NK cells, and one or more cytokine receptor-activating agents which promote the differentiation of Gr-1⁺/CD11b⁺ myeloid progenitor cells into dendritic cells and/or macrophages.

In a specific embodiment, a pharmaceutical composition comprises a pharmaceutical carrier, one or more compounds that activate the IL-12 receptor, one or more compounds that activate the IL-3 receptor, IL-4 receptor, IL-6 receptor, Flt-3, CD40 GM-CSF receptor, M-CSF receptor G-CSF receptor, or CSF receptor, and one or more co-stimulatory molecule-activating agents. In a preferred embodiment, a pharmaceutical composition comprises a pharmaceutical carrier, one or more compounds that activate the IL-12 receptor, one or more compounds that activate the GM-CSF receptor, and one or more compounds that activate 4-1BB. In another preferred embodiment, a pharmaceutical composition comprises a pharmaceutical carrier, one or more compounds that activate the IL-12 receptor, one or more compounds that activate the GM-CSF receptor, and one or more compounds that activate OX40. In yet another preferred embodiment, a pharmaceutical composition comprises a pharmaceutical carrier, one or more compounds that activate the IL-12 receptor, one or more compounds that activate the GM-CSF receptor, one or more compounds that activate 4-1BB, and one or more compounds that activate OX-40.

The present invention provides therapeutic and pharmaceutical compositions comprising a pharmaceutical carrier, one or more cytokine receptor-activating agents, and at least one fusion protein, wherein the fusion protein comprises a co-stimulatory molecule-activating polypeptide fused a heterologous protein, polypeptide or peptide. The present invention provides therapeutic and pharmaceutical compositions comprising a pharmaceutical carrier, one or more co-stimulatory molecule-activating agents, and at least one fusion protein, wherein the fusion protein comprises a cytokine receptor-activating polypeptide fused a heterologous protein, polypeptide or peptide. The present invention further provides therapeutic and pharmaceutical compositions comprising a pharmaceutical carrier and at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. Nucleic acid molecules encoding fusion proteins may be utilized in the therapeutic or pharmaceutical compositions of the invention rather than the fusion proteins themselves.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic tumor-targeted bacteria, preferably attenuated tumor-targeted bacteria, in purified form, and therapeutically effective amounts of one or more immunomodulatory agents, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a suspending agent and a local anesthetic such as lignocaine to ease pain at the site of the injection. Generally, the components are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The present invention provides methods for preventing or treating cancer, an inflammatory disorder, or an infectious disease in a subject, said methods comprising administering to a subject in need thereof an effective amount of one or more cytokine receptor-activating agents and an effective amount of one or more co-stimulatory molecule-activating agents. One or more cytokine receptor-activating agents may be administered to a subject with cancer, an inflammatory disorder or an infectious disease prior to (e.g., 2 minutes, 5 minutes, 10 minutes, 15 minutes, 30 minutes, 45 minutes, 60 minutes, 2 hours, 4 hours, 6 hours, 8 hours, 10 hours, 12 hours, 14 hours, 16 hours, 18 hours, 20 hours, 22 hours, 24 hours, 2 days, 4 days, 5 days, 7 days, 2 weeks, 4 weeks or 6 weeks before), concomitantly with, or subsequent to (e.g., 2 minutes, 5 minutes, 10 minutes, 15 minutes, 30 minutes, 45 minutes, 60 minutes, 2 hours, 4 hours, 6 hours, 8 hours, 10 hours, 12 hours, 14 hours, 16 hours, 18 hours, 20 hours, 22 hours, 24 hours, 2 days, 4 days, 5 days, 7 days, 2 weeks, 4 weeks or 6 weeks after) the administration of one or more co-stimulatory molecule-activating agents.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, and an effective amount of one or more co-stimulatory molecule-activating agents. Preferably, the cytokine receptor-activating agent shifts the Th1/Th2 balance in a subject, and more preferably, the cytokine receptor-activating agent shifts the Th1/Th2 balance and induces the proliferation and/or differentiation of Th1 cells in a subject. In one embodiment, the present invention provides a method for preventing or treating cancer or an infectious disease in a subject comprising administering to said subject an effective amount one or more compounds that activate the IL-15 receptor and an effective amount of one or more co-stimulatory molecule-activating agents. In another embodiment, the present invention provides a method for preventing or treating cancer or an infectious disease in a subject comprising administering to said subject an effective amount one or more compounds that activate the IL-18 receptor and an effective amount of one or more co-stimulatory molecule-activating agents. In yet another embodiment, the present invention provides a method for preventing or treating cancer or an infectious disease in a subject comprising administering to said subject an effective amount one or more compounds that activate Flt3 and an effective amount of one or more co-stimulatory molecule-activating agents. The present invention provides methods for preventing or treating cancer or an infectious disease in a subject, said methods comprising administering to a subject in need thereof an effective amount of a compound that activates the IL-12 receptor (e.g., IL-12 or anti-IL-12R antibodies) and an effective amount of a co-stimulatory molecule-activating agent. In one embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies) and an effective amount of one or more compounds that activate 4-1BB (e.g., 4-1BB ligand or anti-4-1BB antibody). In another embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies) and an effective amount of one or more compounds that activate OX40 (e.g., OX40 ligand or anti-OX40 antibody).

In a preferred embodiment, the present invention provides a method for preventing or treating cancer or an infectious disease in a subject, said method comprising administering to said subject an effective amount of a recombinant adenovirus engineered to express IL-12 and an effective amount of an agonistic anti-4-1BB monoclonal antibody or antigen-binding fragment thereof. In another preferred embodiment, the present invention provides a method for preventing or treating cancer or an infectious disease in a subject, said method comprising administering to said subject an effective amount of a recombinant adenovirus engineered to express IL-12 and an effective amount of an agonistic anti-OX40 monoclonal antibody or antigen-binding fragment thereof.

The present invention provides methods for preventing or treating cancer or an infectious disease in a subject, said methods comprising administering to a subject in need thereof an effective amount of one or more compounds that activate the IL-12 receptor (e.g., IL-12 or anti-IL-12R antibodies) and an effective amount of two or more co-stimulatory molecule-activating agents. In a preferred embodiment, the present invention provides a method for preventing or treating cancer or an infectious disease in a subject, said method comprising administering to said subject an effective amount of one or more compounds that activate the IL-12 receptor (e.g., IL-12 or anti-IL-12R antibodies), an effective amount of one or more compounds that activate 4-1BB (e.g., 4-1BB ligand or anti-4-1BB antibody), and an effective amount of one or more compounds that activate OX40 (e.g., OX40 ligand or anti-OX40 antibody). In another embodiment, the present invention provides a method for preventing or treating cancer or an infectious disease in a subject, said method comprising administering to said subject an effective amount of one or more compounds that activate the IL-12 receptor, an effective amount of one or more compounds that activate 4-1BB, and an effective amount of one or more compounds that activate SLAM, ICOS, B7RP-1 or CD27. In another embodiment, the present invention provides methods for preventing or treating cancer or an infectious disease in a subject, said method comprising administering to said subject an effective amount of one or more compounds that activate the IL-12 receptor, an effective amount of one or more compounds that activate OX40, and an effective amount of one or more compounds that activate SLAM, ICOS, B7RP-1 or CD27. In yet another embodiment, the present invention provides a method for preventing or treating cancer or an infectious disease in a subject, said method comprising administering to said subject an effective amount of one or more compounds that activate the IL-12 receptor, an effective amount of one or more compounds that activates 4-1BB, an effective amount of one or more compounds that activate OX40, and an effective amount of one or more compounds that activate SLAM, ICOS, B7RP-1 or CD27.

In a preferred embodiment, the present invention provides a method for preventing or treating cancer or an infectious disease in a subject, said method comprising administering to said subject an effective amount of a recombinant adenovirus engineered to express IL-12, an effective amount of an agonistic anti-4-1BB monoclonal antibody or antigen-binding fragment thereof, and an effective amount of an agonistic anti-OX40 monoclonal antibody or antigen-binding fragment thereof.

The present invention provides methods for preventing or treating cancer or an infectious disease in a subject, said methods comprising administering to a subject in need thereof an effective amount of two or more compounds that activate the IL-12 receptor, one or more compounds that activate at least one cytokine receptor other than the IL-12 receptor, and an effective amount of one or more co-stimulatory molecule-activating agents. In one embodiment, the present invention provides a method for preventing or treating cancer or an infectious disease in a subject, said method comprising administering to said subject an effective amount of one or more compounds that activate the IL-12 receptor, an effective amount of one or more compounds that activate at least one cytokine receptor other the IL-12 receptor (e.g., one or more cytokines such as IFN-α, IFN-β, IFN-γ, TNF-α, Flt3 ligand, IL-1β, IL-2, IL-3, IL-4, IL-5, IL-6, IL-7, IL-8, IL-9, IL-10, IL-12, IL-15, IL-18, GM-CSF, G-CSF, CSF-1, and M-CSF), and an effective amount of one or more co-stimulatory molecule-activating agents. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-15 receptor, and an effective amount of one or more co-stimulatory molecule-activating agents. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-18 receptor, and an effective amount of one or more co-stimulatory molecule-activating agents.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, and an effective amount of one or more co-stimulatory molecule-activating agents which affect the biological activity (e.g., differentiation, proliferation or effector function) of dendritic cells and/or macrophages. In a specific embodiment, the present invention provides a method for preventing or treating cancer, an inflammatory disorder, or an infectious disease in a subject, said method comprising administering to a subject in need thereof an effective amount of one or more compounds that activate the GM-CSF receptor and an effective amount of one or more compounds that activate CD40. In another embodiment, the present invention provides a method for preventing or treating cancer, an inflammatory disorder, or an infectious disease in a subject, said method comprising administering to a subject in need thereof an effective amount of one or more compounds that activate the GM-CSF receptor and an effective amount of one or more compounds that activate 4-1BB.

The present invention provides methods for preventing or treating cancer, an inflammatory disorder, or an infectious disease in a subject, said methods comprising administering to a subject in need thereof an effective amount of one or more cytokine receptor-activating agents which affect the biological activity (e.g., differentiation, proliferation or effector function) of T helper (Th) cells and/or NK cells, an effective amount of one or more cytokine receptor-activating agents which promote the differentiation of myeloid cells into dendritic cells and/or macrophages, and an effective amount of one or more co-stimulatory molecule-activating agents which affect the biological activity (e.g., differentiation, proliferation or effector function) of dendritic cells and/or macrophages. In one embodiment, the present invention provides a method for preventing or treating cancer, an inflammatory disorder, or an infectious disease in a subject, said method comprising administering to a subject in need thereof an effective amount of one or more compounds that activate the IL-12 receptor, an effective amount of one or more compounds that activate the GM-CSF receptor, and an effective amount of one or more compounds that activate CD40.

The present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of myeloid cells into dendritic cells and/or macrophages. Preferably, the cytokine receptor-activating agent which affects the biological activity of Th cells shifts the Th1/Th2 balance in a subject, and more preferably, the cytokine receptor-activating agent which affects the biological activity of Th cells shifts the Th1/Th2 balance and induces the proliferation and/or differentiation of ThI cells in a subject.

In a preferred embodiment, the present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of Gr-1⁺ myeloid progenitor cells into dendritic cells and/or macrophages. In another preferred embodiment, the present invention provides methods for preventing or treating cancer, an inflammatory disorder, or an infectious disease in a subject, said methods comprising administering to a subject in need thereof an effective amount of one or more co-stimulatory molecule-activating agents, an effective amount of one or more cytokine receptor-activating agents which affect the biological activity (e.g., differentiation, proliferation or effector function) of T helper (Th) cells and/or NK cells, and an effective amount of one or more cytokine receptor-activating agents which promote the differentiation of Gr-1⁺/CD11b⁺ myeloid progenitor cells into dendritic cells and/or macrophages.

In a specific embodiment, the present invention provides a method for preventing or treating cancer or an infectious disease in a subject, said method comprising administering to said subject an effective amount of one or more compounds that activate the IL-12 receptor, an effective amount of one or more compounds that activate the IL-3 receptor, IL-4 receptor, IL-6 receptor, Flt-3, GM-CSF receptor, M-CSF receptor G-CSF receptor, or CSF receptor, and an effective amount of one or more co-stimulatory molecule-activating agents. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the GM-CSF receptor, and an effective amount of one or more compounds that activate 4-1BB. In another embodiment, the present invention provides a method for preventing or treating cancer or an infectious disease in a subject, said method comprising administering to said subject an effective amount of one or more compounds that activate the IL-12 receptor, an effective amount of one or more compounds that activate the GM-CSF receptor, and an effective amount of one or more compounds that activate OX40. In yet another embodiment, the present invention provides a method for preventing or treating cancer or an infectious disease in a subject, said method comprising administering to said subject an effective amount of one or more compounds that activate the IL-12 receptor, an effective amount of one or more compounds that activate the GM-CSF receptor, an effective amount of one or more compounds that activate 4-1BB, and an effective amount of one or more compounds that activate OX-40.

In a preferred embodiment, the present invention provides a method for preventing or treating cancer or an infectious disease in a subject, said method comprising administering to said subject an effective amount of a recombinant adenovirus engineered to express IL-12, an effective amount of a recombinant adenovirus engineered to express GM-CSF, and an effective amount of an agonistic anti-4-1BB monoclonal antibody or antigen-binding fragment thereof. In another preferred embodiment, the present invention provides a method for preventing or treating cancer or an infectious disease in a subject, said method comprising administering to said subject an effective amount of a recombinant adenovirus engineered to express IL-12, an effective amount of a recombinant adenovirus engineered to express GM-CSF, and an effective amount of an agonistic anti-OX40 monoclonal antibody or antigen-binding fragment thereof. In yet another preferred embodiment, the present invention provides a method, for preventing or treating cancer or an infectious disease in a subject, said method comprising administering to said subject an effective amount of a recombinant adenovirus engineered to express IL-12, an effective amount of a recombinant adenovirus engineered to express GM-CSF, an effective amount of an agonisitic anti-4-1BB monoclonal antibody, and an effective amount of an agonistic anti-OX40 monoclonal antibody.

The present invention provides methods for preventing or treating cancer, an inflammatory disorder, or an infectious disease in a subject, said methods comprising administering to a subject in need thereof an effective amount of one or more cytokine receptor-activating agents and an effective amount of at least one fusion protein, wherein the fusion protein comprises a co-stimulatory molecule-activating polypeptide fused a heterologous protein, polypeptide or peptide. The present invention also provides methods for preventing or treating cancer, an inflammatory disorder, or an infectious disease in a subject, said methods comprising administering to a subject in need thereof an effective amount of one or more co-stimulatory molecule-activating agents and an effective amount of at least one fusion protein, wherein the fusion protein comprises a cytokine receptor-activating polypeptide fused a heterologous protein, polypeptide or peptide. Nucleic acid molecules encoding fusion proteins may be administered to a subject with cancer, an inflammatory disorder or an infectious disease rather than the fusion proteins themselves.

The present invention also provides methods for preventing or treating cancer, an inflammatory disorder, or an infectious disease in a subject, said methods comprising administering to a subject in need thereof an effective amount of at least two fusion proteins, wherein one of the fusion proteins comprises a co-stimulatory molecule-activating polypeptide fused a heterologous protein, polypeptide or peptide, and the other fusion protein comprises a cytokine receptor-activating polypeptide fused a heterologous protein, polypeptide or peptide. In a specific embodiment, the present invention provides a method for preventing or treating cancer or an infectious disease in a subject, said method comprising administering to said subject an effective amount of at least two fusion proteins, wherein one of the fusion proteins comprises a cytokine receptor-activating polypeptide that activates the IL-12 receptor fused a heterologous protein, polypeptide or peptide, and the other fusion protein comprises a co-stimulatory molecule-activating polypeptide that activates 4-1BB or OX40 fused a heterologous protein, polypeptide or peptide.

The present invention provides methods for preventing or treating cancer in a subject, said methods comprising administering to a subject in need thereof an effective amount of one or more cytokine receptor-activating agents, an effective amount of one or more co-stimulatory molecule-activating agents, and at least one other known cancer therapy (e.g., radiation therapy or chemotherapy). In a specific embodiment, the present invention provides a method for preventing or treating cancer in a subject, said method comprising administering to said subject an effective amount of one or more cytokine receptor-activating agents, an effective amount of one or more co-stimulatory molecule-activating agents, and an effective amount of at least one other anti-cancer agent such as a chemotherapeutic agent or an antibody that immunospecifically binds to a cancer cell antigen. Examples of chemotherapeutic agents include, but are not limited to, cisplatin, ifosfamide, paclitaxol, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, taxol, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, melphalan, glucocorticoids, procaine, tetracaine, lidocaine, propranolol, puromycin homologs, and cytoxan. Examples of antibodies which can be used in the treatment of cancer include, but are not limited to, Herceptin® (Trastuzumab; Genetech, Calif.) which is a humanized anti-HER2 monoclonal antibody for the treatment of patients with metastatic breast cancer; Retuxan® (rituximab; Genentech) which is a chimeric anti-CD20 monoclonal antibody for the treatment of patients with non-Hodgkin's lymphoma; OvaRex (AltaRex Corporation, MA) which is a murine antibody for the treatment of ovarian cancer; Panorex (Glaxo Wellcome, N.C.) which is a murine IgG₂ₐ antibody for the treatment of colorectal cancer; BEC2 (ImClone Systems Inc., NY) which is murine IgG antibody for the treatment of lung cancer; IMC-C225 (Imclone Systems Inc., NY) which is a chimeric IgG antibody for the treatment of head and neck cancer; Vitaxin (MedImmune, Inc., MD) which is a humanized antibody for the treatment of sarcoma; Campath I/H (Leukosite, Mass.) which is a humanized IgG¹ antibody for the treatment of chronic lymphocytic leukemia (CLL); Smart MI95 (Protein Design Labs, Inc., CA) which is a humanized IgG antibody for the treatment of acute myeloid leukemia (AML); LymphoCide (Immunomedics, Inc., NJ) which is a humanized IgG antibody for the treatment of non-Hodgkin's lymphoma; Smart I D10 (Protein Design Labs, Inc., CA) which is a humanized antibody for the treatment of non-Hodgkin's lymphoma; and Oncolym (Techniclone, Inc., CA) which is a murine antibody for the treatment of non-Hodgkin's lymphoma.

The present invention provides methods for preventing or treating an inflammatory disorder in a subject, said methods comprising administering to a subject in need thereof an effective amount of one or more cytokine receptor-activating agents, an effective amount of one or more co-stimulatory molecule-activating agents, and at least one other known anti-inflammatory agent. Examples of anti-inflammatory agents include, but are not limited to, aspirin, non-steroidal anti-inflammatory agents (e.g. , ibuprofen, fenoprofen, indomethacin, and naproxen), Cox-2 inhibitors (e.g., rofecoxib (Vioxx) and celecoxib (Celebrex)), and anti-TNFα agents (e.g., infliximab (Remicade) and etanercept (Enbrel)).

The present invention provides methods for preventing or treating an infectious disease in a subject, said methods comprising administering to a subject in need thereof an effective amount of one or more cytokine receptor-activating agents, an effective amount of one or more co-stimulatory molecule-activating agents, and at least one known anti-viral, anti-microbial agent or anti-fungal agent. Examples of antibodies used as anti-viral or anti-microbial agents for the treatment of viral infection or microbial infection include, but are not limited to, PRO542 (Progenies) which is a CD4 fusion antibody for the treatment of HIV infection; Ostavir (Protein Design Labs, Inc., CA) which is a human antibody for the treatment of hepatitis B virus; Protovir (Protein Design Labs, Inc., CA) which is a humanized IgG₁ antibody for the treatment of cytomegalovirus (CMV); and anti-LPS antibodies. Examples of antibiotics used as anti-microbial agents for the treatment of microbial infections include, but are not limited to, penicillin, amoxicillin, ampicillin, carbenicillin, ticarcillin, piperacillin, cepalospolin, vancomycin, tetracycline, erythromycin, amphotericin B, nystatin, metronidazole, ketoconazole, and pentamidine. Examples of drugs used for the treatment of viral infections include, but are not limited to, inhibitors of reverse transcriptase (e.g., AZT, 3TC, D4T, ddC, ddI, d4T, 3TC, adefovir, efavirenz, delavirdine, nevirapine, abacavir, and other dideoxynucleosides or dideoxyfluoronucleosides); inhibitors of viral mRNA capping, such as ribavirin; inhibitors of proteases such HIV protease inhibitors (e.g., amprenavir, indinavir, nelfinavir, ritonavir, and saquinavir,); amphotericin B; castanospermine as an inhibitor of glycoprotein processing; inhibitors of neuraminidase such as influenza virus neuraminidase inhibitors (e.g., zanamivir and oseltamivir); topoisomerase I inhibitors (e.g., camptothecins and analogs thereof); amantadine; and rimantadine.

The amount of a cytokine receptor-activating agent, co-stimulatory molecule-activating agent or pharmaceutical composition which will be effective in the prevention or treatment of a disease or disorder will depend on the nature of the disease or disorder and the overall state of the subject, and can be determined by standard clinical techniques. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For cytokine receptor-activating polypeptides and co-stimulatory molecule-activating polypeptides, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration (e.g., into the dermis) of the antibodies by modifications such as, for example, lipidation.

For cytokine receptor-activating agents and co-stimulatory molecule-activating agents which are small molecules the appropriate doses will vary depending upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight, e.g., about 1 microgram per kilogram to about 500 milligrams per kilogram, about 100 micrograms per kilogram to about 5 milligrams per kilogram, or about 1 microgram per kilogram to about 50 micrograms per kilogram. Small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Various delivery systems are known and can be used to administer a cytokine receptor-activating agent, co-stimulatory molecule-activating agent or pharmaceutical composition, e.g., encapsulation in liposomes, microparticles, microcapsules, recombinant cells capable of expressing a cytokine receptor-activating polypeptide or a co-stimulatory molecule-activating polypeptide, receptor-mediated endocytosis (see, e.g., Wu and Wu, J. Biol. Chem. 262:4429-4432 (1987)), construction of a nucleic acid as part of a retroviral or other vector, etc. Cytokine receptor-activating agents, co-stimulatory molecule-activating agents and/or pharmaceutical compositions may administered to a subject, e.g., intradermally, intramuscularly, intraperitoneally, intravenously, subcutaneously, intranasally, topically, intratumorally, intrathecally, epidurally, or orally. Cytokine receptor-activating agents, co-stimulatory molecule-activating agents, and pharmaceutical compositions may be administered by any convenient route, for example by infusion or bolus injection, by absorption through epithelial or mucocutaneous linings (e.g., oral mucosa, rectal and intestinal mucosa, etc.) and may be administered together with other biologically active agents such as, e.g., chemotherapeutic agents. Further, cytokine receptor-activating agents, co-stimulatory molecule-activating agents and/or pharmaceutical compositions may be administrated to a subject systemically or locally.

For administration by inhalation, cytokine receptor-activating agents, co-stimulatory molecule-activating agents and/or pharmaceutical compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In a specific embodiment, it may be desirable to administer the cytokine receptor-activating agents, co-stimulatory molecule-activating agents and/or pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a cytokine receptor-activating agent, co-stimulatory molecule-activating agent and/or pharmaceutical composition, care must be taken to use materials to which the cytokine receptor-activating agent, co-stimulatory molecule-activating agent and/or pharmaceutical composition does not absorb.

In another embodiment, a cytokine receptor-activating agent, co-stimulatory molecule-activating agent and/or pharmaceutical composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.).

In yet another embodiment, a a cytokine receptor-activating agent, co-stimulatory molecule-activating agent and/or pharmaceutical composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938,. PCT publication WO 91/05548, PCT publication WO 96/20698,. Ning et al., 1996, “Intratumoral Radioimmunotheraphy of a Human Colon Cancer Xenograft Using a Sustained-Release Gel,” Radiotherapy & Oncology 39:179-189,. Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

5.6.1. Gene Therapy

In one embodiment, one or more nucleic acid molecules comprising sequences encoding one or more cytokine receptor-activating polypeptides and/or one or more nucleic acid molecules comprising sequences encoding one or more co-stimulatory molecule-activating polypeptides are administered to a subject to prevent or treat cancer, an inflammatory disorder or an infectious disease, by way of gene therapy. In a specific embodiment, one or more nucleic acid molecules encoding one or more cytokines (e.g., IL-12, IL-15, IL-18, Flt3 ligand, or GM-CSF), derivatives, analogs or functional fragments thereof are administered to a subject to prevent or treat cancer, an inflammatory disorder or an infectious disease, by way of gene therapy. In another embodiment, one or more nucleic acid molecules encoding one or more agonistic antibodies immunospecific for one or more cytokine receptors (e.g., the IL-12 receptor, IL-15 receptor, IL-18 receptor, Flt3 or GM-CSF receptor) are administered to a subject to prevent or treat cancer, an inflammatory disorder or an infectious disease, by way of gene therapy. In another embodiment, one or more nucleic acid molecules encoding one or more ligands immunospecific for one or more co-stimulatory molecules selectively expressed by activated immune cells (preferably, activated T-cells) are administered to a subject to prevent or treat cancer, an inflammatory disorder or an infectious disease, by way of gene therapy. In yet another embodiment, one or more nucleic acid molecules encoding one or more agonistic antibodies immunospecific for one or more co-stimulatory molecules selectively expressed by activated immune cells (preferably, activated T-cells) are administered to a subject to prevent or treat cancer, an inflammatory disorder or an infectious disease, by way of gene therapy. Gene therapy refers to therapy performed by the administration to a subject of an expressed or expressible nucleic acid.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, a composition of the invention comprises nucleotide sequences encoding one or more cytokine-receptor activating polypeptides and/or one or more co-stimulatory molecule-activating polypeptides, said nucleic acid sequences being part of expression vectors that express cytokine-receptor activating polypeptides and/or co-stimulatory molecule-activating polypeptides in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the cytokine-receptor-activating polypeptide and/or co-stimulatory molecule-activating polypeptide coding sequences and any other desired sequences are flanked by regions that promote homologous recombination at a desired site in the genome, thus providing for intrachromosomal expression of the cytokine-receptor-activating polypeptide and/or co-stimulatory molecule-activating polypeptide nucleic acids (Koller and Smithies, 1989, Proc. Natl. Acad. Sci. USA 86:8932-8935; Zijlstra et al., 1989, Nature 342:435-438).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g, PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In one embodiment, viral vectors that contain nucleic acids encoding one or more cytokine receptor-activating polypeptides and/or one or more co-stimulatory molecule-activating polypeptides are used in accordance with the invention (see Miller et al., 1993, Meth. Enzymol. 217:581-599). In a specific embodiment, viral vectors that contain nucleotide sequences encoding one or more cytokines (e.g., IL-12, IL-15, IL-18 or GM-CSF) or one or more agonistic antibodies immunospecific for one or more cytokine receptors (e.g., the IL-12 receptor, IL-15 receptor, IL-18 receptor and GM-CSF receptor) are used in accordance with the invention. In another embodiment, viral vectors that contain nucleotide sequences encoding one or more ligands or one or more agonistic antibodies immunospecific for co-stimulatory molecules selectively expressed by activated immune cells are used in accordance with the invention.

A retroviral vector, for example, can be used in gene therapy to deliver a cytokine receptor-activating polypeptide or co-stimulatory molecule-activating polypeptide to a subject. These retroviral vectors have been modified to delete retroviral sequences that are not necessary for packaging of the viral genome and integration into host cell DNA. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783. In a preferred embodiment, adenovirus vectors are used in gene therapy to deliver cytokine-receptor-activating polypeptides and/or co-stimulatory molecule-activating polypeptides to a subject to prevent or treat cancer, an inflammatory disorder or an infectious disease.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include, but are not limited to, epithelial cells, endothelial cells, keratinocytes, fibroblasts, muscle cells, hepatocytes; blood cells such as T lymphocytes, B lymphocytes, NK cells, dendritic cells, monocytes, macrophages, neutrophils, eosinophils, megakaryocytes, granulocytes; various stem or progenitor cells, in particular hematopoietic stem or progenitor cells, e.g, as obtained from bone marrow, umbilical cord blood, peripheral blood, fetal liver, etc.

In a preferred embodiment, the cell used for gene therapy is autologous to the patient.

In one embodiment in which recombinant cells are used in gene therapy, one or more nucleotide sequences encoding one or more cytokine receptor-activating polypeptides and/or one or more nucleotide sequences encoding one or more co-stimulatory molecule-activating polypeptides are introduced into the cells such that the nucleotide sequences are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.8. Methods of Determining the Prophylactic or Therapeutic Utility

Several aspects of the pharmaceutical compositions or compounds of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition or compound is indicated, include cell culture asssays in which a patient tissue sample is grown in culture, and exposed to or otherwise contacted with a pharmaceutical composition or compound, and the effect of such composition or compound upon the tissue sample is observed. The tissue sample can be obtained by biopsy from the patient. This test allows the identification of the therapeutically most effective composition or compound for each individual patient. In various specific embodiments, in vitro assays can be carried out with representative cells of cell types involved in cancer, an infectious disease, or an inflammatory disorder (e.g., T cells), to determine if a pharmaceutical composition or compound of the invention has a desired effect upon such cell types.

Cytokine receptor-activating agents and/or co-stimulatory molecule-activating agents can be tested for their ability to augment activated immune cells by contacting activated immune cells with a test compound or a control compound and determining the ability of the cytokine receptor-activating agents and/or co-stimulatory molecule-activating agents to modulate (e.g., increase) the biological activity of the activated immune cells. The ability of a cytokine receptor-activating agents and/or co-stimulatory molecule-activating agents to modulate the biological activity of activated immune cells can be assessed by detecting the expression of cytokines or antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by ³H-thymidine incorporation assays and trypan blue cell counts. Cytokine and antigen expression can be assayed, for example, by immunoassays including, but are not limited to, competitive and non-competitive assay systems using techniques such as western blots, immunohisto-chemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electromobility shift assays (EMSAs). The effector function of T-cells can be measured, for example, by a ⁵¹Cr-release assay (see, e.g., Palladino et al., 1987, Cancer Res. 47:5074--5079 and Blachere et al., 1993, J. Immunotherapy 14:352-356).

Combinations of cytokine receptor-activating agents and/or co-stimulatory molecule-activating agents can be tested in suitable animal model systems prior to use in humans. Such animal model systems include, but are not limited to rats, mich, chicken, cows, monkeys, pigs, dogs, rabbits, etc. Any animal system well-known in the art may be used. In a specific embodiment of the invention, combinations of cytokine receptor-activating agents and/or co-stimulatory molecule-activating agents are tested in a mouse model system. Such model systems are widely used and well-known to the skilled artisan. Cytokine receptor-activating agents and/or co-stimulatory molecule-activating agents can be administered repeatedly. Several aspects of the procedure may vary. Said aspects include the temporal regime of administering the cytokine receptor-activating agents and/or co-stimulatory molecule-activating agents and whether such agents are administered separately or as a admixture.

The anti-inflammatory activity of the combination therapies of the invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumtology, McCarty et al.(eds.), Chapter 30 (Lee and Febiger, 1993). Experimental and spontaneous animal models of inflammatory arthritis and autoimmune rheumatic diseases can also be used to assess the anti-flammatory activity of the combination therapies of the invention. The following are some assays provided as examples and not by limitation. The principle animal models for arthritis or inflammatory disease known in the art and widely used include: adjuvant-induced arthritis rat models, collagen-induced arthritis rat and mouse models and antigen-induced rat, rabbit and hamster models, all described in Crofford L. J. and Wilder R. L., “Arthritis and Autoimmunity in Animals”, in Arthritis and Allied Conditions: A Textbook of Rheumtology, McCarty et al. (eds.), Chapter 30 (Lee and Febiger, 1993), incorporated herein by reference in its entirety.

The anti-inflammatory activity of the combination therapies of invention can be assessed using a carrageenan-induced arthritis rat model. Carrageenan-induced arthritis has Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the combination therapies of invention can be also be assessed by measuring the inhibition of carrageenan-induced paw edema in the rat, using a modification of the method described in Winter C. A. et al., “Carrageenan-Induced Edema In Hing Paw of the Rat as an Assay for Anti-inflammatory Drugs” Proc. Soc. Exp. Biol Med. 111, 544-547, (1962). This assay has been used as a primary in vivo screen for the anti-inflammatory activity of mos NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the combination therapies of invention. Additionally, animal models for inflammatory bowel disease can also be used to assess the efficacy of the combination therapies of invention (Kim et al., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of the combination therapies of the invention. An example of one such model is the murine adoptive transfer model in which aeroallergen provocation of Th1 or Th2 recipient mice results in TH effector cell migration to the airways and is associated with an intense neutrophilic (TH1) and eosinophilic (TH2) lung mucosal inflammatory response (Cohn et al., 1997, J. Exp. Med. 186:1737-1747).

Animal models for cancer or an infectious disease can also be used to assess the efficacy of the combination therapies of invention. Any animal model for cancer or an infectious disease well-known to one of skill in the art can be used to assess the efficacy of the combination therapies of the invention.

Cytokine receptor-activating agents and/or co-stimulatory molecule-activating agents can be tested for their ability to reduce tumor formation in patients (i.e., animals) suffering from cancer. Cytokine receptor-activating agents and/or co-stimulatory molecule-activating agents can also be tested for their ability to reduce viral load or bacterial numbers patients suffering from an infectious disease. Cytokine receptor-activating agents and/or co-stimulatory molecule-activating agents can also be tested for their ability to alleviate of one or more symptoms associated with cancer or an infectious disease. Cytokine receptor-activating agents and/or co-stimulatory molecule-activating agents can also be tested for their ability to decrease the time course of the infectious disease. Cytokine receptor-activating agents and/or co-stimulatory molecule-activating agents can also be tested for their ability to decrease or reduce the inflammation of the joints and/or organs of patients with an inflammatory disorder. Further, cytokine receptor-activating agents and/or co-stimulatory molecule-activating agents can be tested for their ability to increase the survival period of patients suffering from cancer or an infectious disease. Techniques known to those of skill in the art can be used to analyze test to function of the test compounds in patients.

Cytokine receptor-activating agents and/or co-stimulatory molecule-activating agents of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Cytokine receptor-activating agents and/or co-stimulatory molecule-activating agents that exhibit large therapeutic indices are preferred. While Cytokine receptor-activating agents and/or co-stimulatory molecule-activating agents that exhibit toxic side effects may be used, care should be taken to design a delivery system that targets such agents to the site of the affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this rage depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.9. Kits

The invention provides a pharmaceutical pack or kit comprising one or more containers with one or more of the components of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In accordance with the invention, any cytokine receptor-activating agent and/or co-stimulatory molecule-activating agent described herein or well-known to one of skill in the art can be incorporated into a kit of the invention. In a specific embodiment of the invention, the kit comprises one or more cytokine receptor-activating agents in a first vial, one or more co-stimulatory molecule-activating agents in a second vial, and optionally means of administering the agents to a subject in need thereof. In another embodiment of the invention, the kit comprises one or more cytokine receptor-activating agents in a first vial, one or more co-stimulatory molecule-activating agents a second vial, one or more anti-cancer therapies, antibiotics, anti-viral agents, anti-fungal agents or anti-inflammatory agents described herein or well-known in the art and optionally, means of administering the agents to a subject in need thereof. The kit may further comprises instructions for use of cytokine receptor-activating agents and co-stimulatory molecule-activating agents. In another embodiment, a kit of the invention comprises a cytokine receptor-activating agent contained in a first vial, a co-stimulatory molecule-activating agent contained in a second vial, and instructions for administering the cytokine receptor-activating agent and the co-stimulatory molecule-activating agent to a subject with cancer, an inflammatory disorder or an infectious disease. In certain embodiments of the invention, the kit comprises a document providing instructions for the use of the composition of the invention in, e.g., written and/or electronic form. Said instructions provide information relating to, e.g., dosage, method of administration, and duration of treatment.

The kits of the invention may also comprise, means of testing the effectiveness of the cytokine receptor-activating agents, co-stimulatory molecule-activating agents, or pharmaceutical compositions of the invention. Said means of testing the effectiveness of the cytokine receptor-activating agents, co-stimulatory molecule-activating agents, or pharmaceutical compositions of the invention include, but are not limited to, cell lines (e.g., tumorigenic cell lines), means of conducting a biopsy procedure, means for administering cell lines to an animal model, means for measuring replication cells or infectious agents (e.g., viruses, fungus or bacteria), means for testing the expression of therapeutic molecules or molecular markers (e.g., antibodies and probes for in situ hybridization), means for testing the infiltration of inflammatory cells, means for testing the differentiation of cells, means for testing the activity state of the immune system, etc. Optionally, associated with such a kit can be a description of how to conduct said tests.

The following series of examples are presented by way of illustration and not by way of limitation of the scope of the invention.

### 6. EXAMPLE

### Rejection of Hepatic Colon Carcinoma and Lung Metastases by Immunomodulatory Therapy with 4-1BB Ligand or Anti-4-1BB and IL-12

The effectiveness of therapeutic compositions comprising IL-12 in combination with 4-1BB ligand or anti-4-1BB in the long-term survival of animal models of liver and macroscopic lung metastatic tumors were evaluated according to the experimental design described below.

6.1 Materials and Methods

Tumor Model and Therapeutic Protocols

MCA26 is a tumor cell line of chemically induced colon carcinoma in BALB/c mouse (Corbett et al., 1975). Metastatic colon cancer was induced by implanting 7×10⁴ MCA26 cells into the left lobe of the liver of 8-10 week old female BALB/c mice (Taconic). At day 7, mice with 5×5 mm² size tumors were selected and different doses of Adv.mIL-12 or control DL312 vector were injected intratumorally in a 50 μl volume of 10 mM Tris-HCl (pH 7.4)/1 mM MgC12/10% (vol/vol) glycerol/Polybrene (20 μg/ml). At day 8 and day 11, 50 μg anti-4-1BB antibody or control rat Ig was administered intraperitoneally (i.p.).

JC cell line is a chemically induced breast carcinoma line derived from a BALB/c background. JC cells are grown and maintained in MEM supplemented with 10% fetal calf serum, 2 mM L-glutamine, 100-unit/ml penicillin, and 100-mg/ml streptomycin. Using the same model of liver metastases as previously described, the left lateral lobe of the liver of adult inbred female BALB/c mice (8- to 10-week old, 18- to 20-g) was injected directly with 1×10⁵ JC cells suspended in 10-μl Hank's balanced salt solution. Ten days after injection, liver tumors measuring between 5×5 mm in diameter were directly injected with ADV/IL-12 or a control vector ADV/DL312 (1×10⁸ pfu/animal). Animals attributed to the gene therapy group received intra-tumoral delivery of ADV/4-1BBL or ADV/DL312 (1×10⁹ pfu/animal) in combination to ADV/IL-12. If assigned to the antibody treatment group, 50 μg of monoclonal anti-4-1BB agonistic antibody or control rat IgG were injected i.p. on day one and three after intratumoral gene treatment with ADV/IL-12. Long-term survival studies were performed to assess treatment outcome.

Rechallenge Test and in vivo Depletion of Lymphocytes

About 100 days after the treatment, the original tumors in survival mice were eradicated. The rechallenge test was performed by implanting 7×10⁴ MCA26 cells subcutaneously on the shaved flanks of the survival animals. Alternatively, the rechallenge test was performed by implanting JC parental tumor cells (1×10⁵) and MCA26 cells (7×10⁴) subcutaneously (s.c.) on left and right flanks, respectively, of mice that survived long-term (>120 days) after ADV/IL-12 plus ADV/4-1BBL, ADV/IL-12+anti-4-1BB, ADV/IL-12 or anti-4-1BB alone treatment., respectively. Animals were observed for tumor formation and rate of tumor growth. “Naive” BALB/c mice that have never been exposed to JC or MCA26 cells were used to assess the normal growth of a s.c. JC or MCA26 tumor.

For in vivo depletion of T-cells (CD8+) or NK cells, either purified ascites from 2.43 hybridoma (ATCC) or polyclonal antibodies anti-asialo GM1 (Wako Co.) or appropriate Ig controls was injected intraperitoneally under established procedures (Brunda et al., 1993, J. Leukocyle Biology 55: 280-288; Colombo et al., 1996, Cancer Res. 56: 2531-2534; Nishmura et al., 1995, Immunology Letters 48: 167-174; Scott 1993, Science 260: 496-497; and Takeda et al., 1996, J. Immunology 156: 3366-3373). The mice were given 2 mg of antibody i.p. per day, beginning one day prior to tumor rechallenge. Antibodies for control and NK+ depletion were administered for five consecutive days then every five days afterward (day −1, 0, 1, 2, 3, 8, 13), while antibodies for CD8+ depletion were given on every other day for three times and then every five days afterward (day −1, 1, 3, 8, 13) according to established optimal conditions. Treatment efficiencies with these antibodies were confirmed by flow cytometry, and effectively depleted subsets (>99%) of the immune lymphocytes were routinely obtained.

Construction of the IL-12 Virus Vector

A recombinant adenovirus expressing mIL-12 was constructed by replacing the E1A region of adenovirus type 5 with an expression cassette pAd/RSV-mIL-12 containing two IL-12 CDNA subunits, p35 and p40, linked by an internal ribosomal entry site (IRES) of the encephalomyocarditis virus (Banks et al., 1995, Br. J Cancer 71: 655-659; Brunda 1994, J. Leukocyte Biology 55: 280-288; Brunda et al., 1993, J Exp. Med. 178:1223-1230; Caruso et al., 1996, Proc. Natl. Acad. Sci., USA 93: 11302-11306; Chen et al., 1997, J. Immunology 159: 351-359; and Tahara et al., 1995, J. Immunology 154: 6466-6474). The recombinant virus was generated by cotransfection with pAd/RSV-mIL-12 and pBHG10 into 293 cells using calcium phosphate precipitation method. Large scale production of recombinant adenovirus was accomplished in 293 cells and purified by double cesium chloride gradient ultracentrifugation. The viral titer [plaque forming units (pfu)/ml] was determined by plaque assay in the 293 cells (Caruso et al., 1996, Proc. Natl. Acad. Sci., USA 93: 11302-11306; and Chen et al., 1997, J. Immunology 159: 351-359). Bioactivity was determined by ELISA for the IFNγ release from naive mouse splenocytes cocultured with supernatant from Adv.mIL-12 transduced (1000 m.o.i.) MCA26 tumor cells.

Construction and Characterization of the Recombinant Adenoviral Vector Expressing 4-1BB Ligand

The full-length mouse 4-1BB ligand cDNA was obtained from pLXSHDm41BB-ligand by PCR amplification using appropriate primers with EcoR V and Not I linkers. The cDNA clone with the correct sequence was subcloned into the adenoviral backbone vector (pAd1.1/RSV-bpA) under the RSV-LTR promoter control at the Not I and EcoR V sites. Recombinant adenovirus was generated by cotransfection with this plasmid with pJM 17 into 293 cells. The positive plaques were purified and further characterized by FACSCAN analysis. Expression of 4-1BB ligand on the Adv.RSV-4-1BB-ligand transduced plasmacytoma cells was highly positive (71% and mean 15) and there was no significant increase in the control vector transduced cells.

Detection of IFN-γ Concentration in the Serum

The mice blood was collected by cutting the tail tips of the treated animals at various time points. Serum was then separated by centrifugation. The IFN γ concentration in the mouse serum was detected by ELISA (R&D Inc.).

In vitro Cytotoxic Assay

Freshly isolated effector cells were analyzed by both CTL and NK cytolytic assays. While CTL assay required an additional stimulation of the effector cells (6×10⁶) with irradiated parental tumor (5×10⁵ cells, receiving 15,000 rads) and recombinant mIL-2 (20 units/ml for 5 days, the NK cytolytic assay directly used freshly isolated MNC to coincubate with ⁵¹Cr labeled target cells (150 μCi/5×10⁶ cells) for 4 hours at 37° C. at various effector to target cell ratios. After incubation, the radioactivity released in the supernatant was measured in a gamma counter. The percentage of cell lysis was calculated as: (experimental release-spontaneous release)/(maximal release-spontaneous release)×100. The standard deviation for the triplicate wells is less than 7%.

In vitro Cell Depletion and Blocking

In vitro depletion of T cell and NK was accomplished by using Thy1.2 hybridoma supernatant (ATCC) and purified DX5 or anti-asialo GM1 antibody (Pharmigen and Wako Co., respectively). The effector cells were incubated with proper concentration of antibodies on ice for 45 minutes and depleted with rabbit complement (Pel-Freez) for two 30 minutes cycles at 37° C. The complement to the effector cells alone did not affect target cell lysis. Optimal concentration of antibodies and complement were used and verified by flow cytometry. There were less than 1% CD3 positive cells present after Thy1.2 T cell depletion. The efficacy of the NK depletion procedure was confirmed by a direct cytolytic assay against YAC-1 using splenocytes from Poly I:C treated animals. In vitro blocking of CD3+ effector population was accomplished by using purified 145-2C11 (Pharmingen). The cells were blocked with 2 μg/1×10⁶ cells for 45 minutes prior to incubating with the target cells.

Macroscopic Metastatic Tumor Model

In order to evaluate the systemic anti-tumor effect of the new combination therapy, a 9 day pre-existing macroscopic metastases model was established. Briefly, 3×10⁴ MCA26 cells were injected through the tail veins one day prior to the usual liver tumor implantation. After eight days, the animals were divided into two groups, one to receive the combination therapy and the other to receive no treatment. On the day of virus injection, several mice were sacrificed for biopsy and pathological observation. 100-200 tumor modules could be observed on the lung surfaces, with sizes ranging from 0.5-0.8 mm in diameter. There were also many nodules present on the walls of gastrointestinal tract and lymph node.

6.2. Results

Anti-4-1BB Antibodies Significantly Enhance the Anti-Tumor Effect of IL-12 Gene Therapy

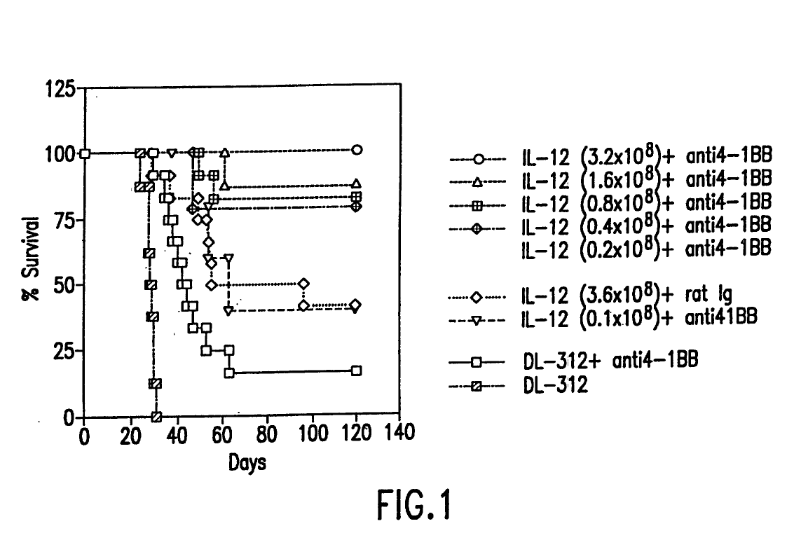

Intratumorally administered Adv.mIL-12 was found to significantly prolong the median survival time of tumor bearing animals, with 25% of the animal becoming tumor free after a single treatment. In an attempt to improve this long-term anti-tumor effect mediated by IL-12, Adv.mIL-12 gene therapy was combined with an agonistic anti-4-1BB antibody administered intraperitoneally. After 120 days, the long-term survival of mice intrahepatically implanted with 7×10⁴ MCA tumor cells and treated with ADV.mIL-12+anti-4-1BB antibody, ADVmIL-12+control antibody, control vector (DL312)+anti-4-1BB antibody, or control vector (DL312) alone was determined. 80-100% of mice in receiving the combination of ADV.mIL-12 and anti-4-1BB antibody remained alive, at Adv.mIL-12 doses ranging from 0.2×10⁸ to 3.6×10⁸ ADV.mIL-12 pfu/mouse (FIG. 1). Only 42.8% of the animals treated with 3.6×10⁸ pfu of Adv.mIL-12+control Ig survived as compared to 100% survival at this dose of Adv.mIL-12+anti-4-1BB, and only 14.5% of control vector (DL312)+anti-4-1BB treated animals survived. Thus, the therapeutic effect of combination therapy (0.2×10⁸−3.6×10⁸ pfu of Adv.mIL-12+anti4-1BB) is significantly better than either treatment alone (p<0.0001).

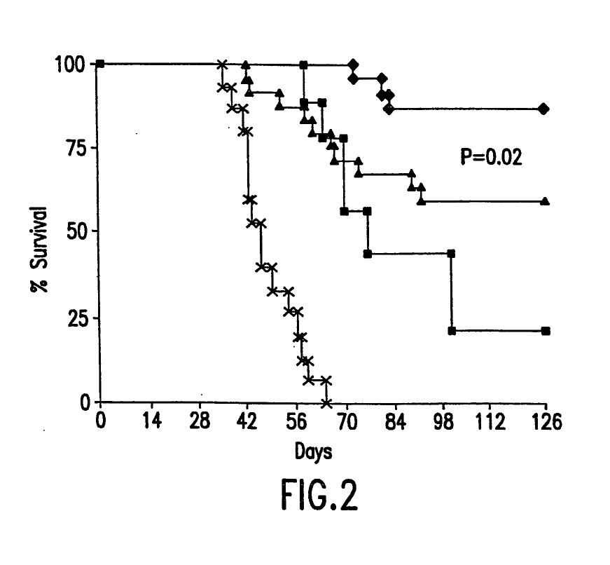

Further, the long-term survival of BALB/c mice bearing JC breast carcinoma liver metastases treated 87, 60, and 22% of tumor bearing mice treated with IL-12+anti-4-1BB, DL312 control vector +anti-4-1BB, or IL-12+rat Ig was evaluated (FIG. 2). 87%, 60%, and 22% of tumor bearing mice treated with IL-12+anti-4-1BB, DL312 control vector +anti-4-1BB, or IL-12+rat Ig, respectively, showed long-term survival (P=0.02 (IL-12+anti-4-1BB versus DL312+anti-4-1BB; P<0.0001 (IL-12+anti-4-1BB versus IL-12+rat Ig); P=0.129 (DL312+anti-4-1BB versus IL-12+rat Ig); logrank test). All mice in the control group died within 60 to 70 days after tumor cell inoculation (P<0.0001 comparing all groups with DL312+rat Ig; logrank test). Thus, the therapeutic effect of IL-12 plus anti-4-1BB antibody results in a better method of treating tumors than IL-12 or anti-4-1BB antibody alone.

4-1BB Ligand can Mimic the Agonistic Antibody and Achieve the Synergistic Effect with Adv.mIL-12 Mediated Gene Therapy

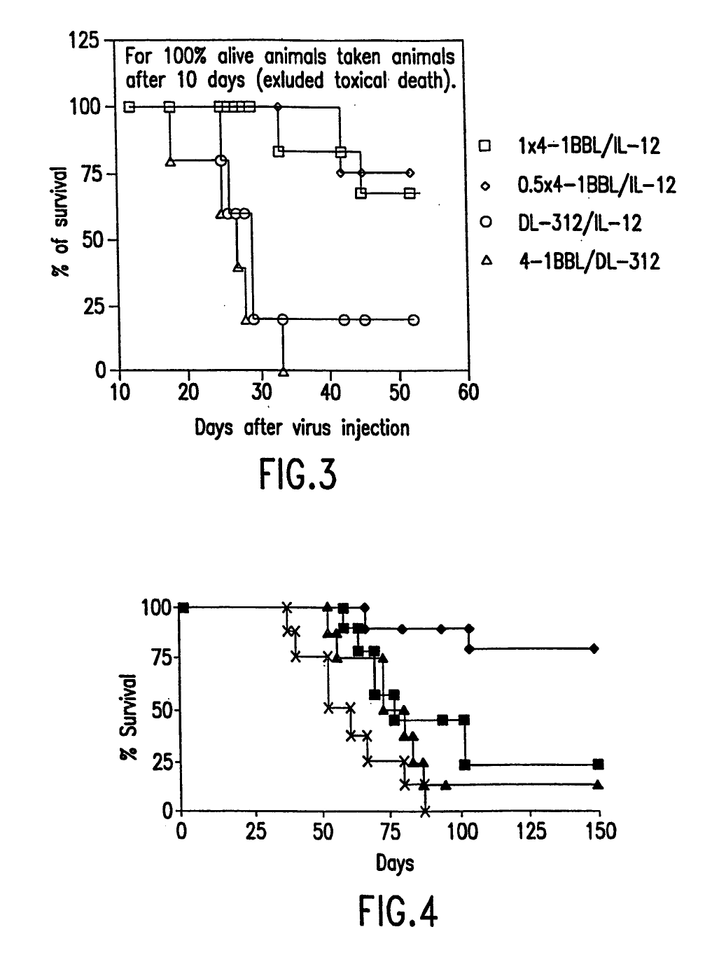

To determine whether the anti-4-1BB antibody can be replaced with a recombinant 35 adenoviral vector expressing 4-1BB ligand, recombinant adenoviral vectors expressing 4-1BB ligand and Adv.mIL-12 were co-delivered at the tumor site. The recombinant adenoviral vector expressing 4-1BB ligand was injected into pre-established hepatic MCA 26 tumors at 1×10⁹ or 5×10⁸ pfu/animal alone and/or a sub-optimal dose of the Adv.mIL-12 vector at 2×10⁸ pfu/mouse. All animals treated with the control vector and 4-1BB ligand died within 32 days (FIG. 3). The medium survival rate for Adv.mlL-12 was only 28 days. However, the combined application of Adv.4-1BB ligand and Adv.mIL-12 resulted in longer survival than either treatment alone (p<0.042). The results indicate that 4-1BB ligand and mIL-12 vectors have together generated an effective anti-tumor immunity in mice with pre-established hepatic MCA 26 tumors.

To determine whether the effect of 4-1BB ligand treatment in combination with mIL-12 treatment results in long-term survival in mice having other types of tumors, mice having pre-established JC breast carcinoma liver metastases were analyzed for long-term survival (FIG. 4). 78%, 22%, and 13% of animals receiving IL-12-12+4-1BBL, IL-12+DL312, and 4-1BBL +DL312, respectively, were long-term survivors (P=0.016 (IL-12+4-1BBL versus IL-12+DL312); P=0.004 (IL-12+4-1BBL versus 4-1BBL+DL312); P=0.515 (IL-12 versus 4-1BBL); logrank test). All animals in the control group died within 80 to 90 days (P=0.0002 (IL-12+4-1BBL); P=0.011 (IL-12); P=0.132 (4-1BBL); logrank test). These results confirm that the combination of 4-1BB ligand and mIL-12 vectors together result in a more effective anti-tumor immunity than either 4-1BB ligand or mIL-12 alone.

Challenge Experiments with Parental Tumor Cells

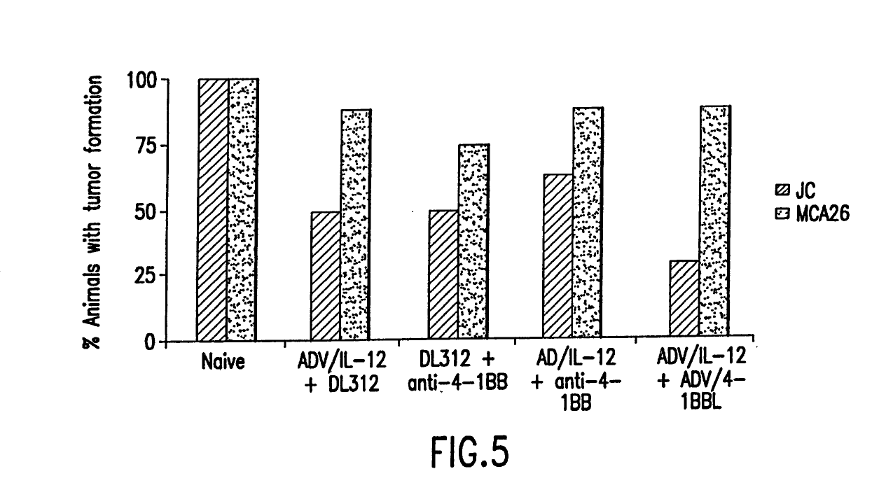

The persistence of systemic anti-tumor immunity was tested in long-term (>120 days) surviving animals after ADV/IL-12+ADV/4-1BBL, ADV/IL-12+anti-4-1BB, ADV/IL-12 or anti-4-1BB alone treatment. JC parental tumor cells (1×10⁵) and MCA26 cells (7×10⁴) were implanted subcutaneously (s.c.) on left and right flanks, respectively. Animals were observed for tumor formation and rate of tumor growth. “Naive” BALB/c mice that have never been exposed to JC or MCA26 cells were used to assess the normal growth of a s.c. JC or MCA26 tumor. All naive animals grew s.c. JC or MCA26 tumors. 29% of IL-12+4-1BBL, 50% of IL-12+DL312 or anti-4-1BB+D1 312, and 63% of IL-12+anti-4-1BB treated animals formed a JC tumor (FIG. 5). Compared to naive animals, only the results of IL-12+4-1BBL group are significant (P=0.007, Fischer's exact test). However, the rate of JC tumor growth in each long-term surviving animal was dramatically decreased in comparison to naive controls.

Rejection of Macroscopic Lung Metastases of Colon Carcinoma after Combination Treatment in Animals with Hepatic Tumors

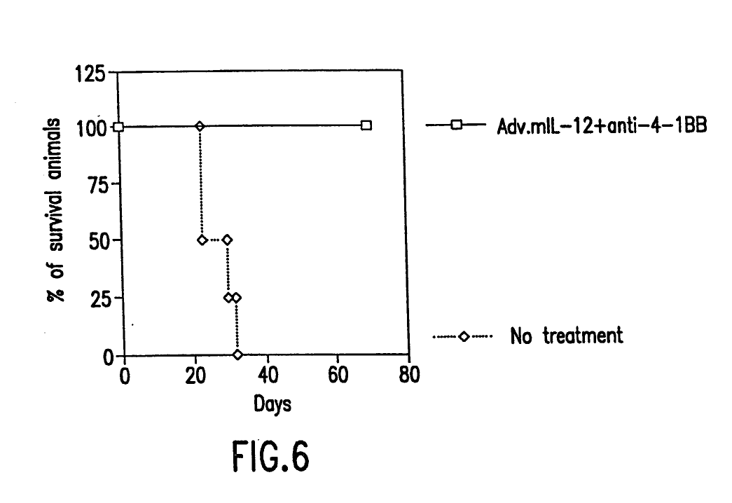

Rejection of macroscopic lung metastases of colon carcinoma after combination therapy, an animal model with pre-established multiple macroscopic tumor nodules in the lung that range from 0.5 to 0.8 mm in diameter was subjected to the test. Animals receiving tail vein infusion of 3×10⁴ MCA26 cells developed multiple lesions in the lung, and 100% of them die within 32 days. However, all the liver and lung tumor bearing animals receiving the combination treatment in the liver tumor survived well after 70 days (FIG. 6). The results strongly suggest that systemic anti-tumor immunity generated from the combination therapy was capable of eradicating pre-existing metastatic tumor in distant organs. (p<0.0011) by logrank test.

Anti-4-1BB Antibodies and Adv.mIL-12 Synergistically Activate Anti-Tumor Natural Killer Cells

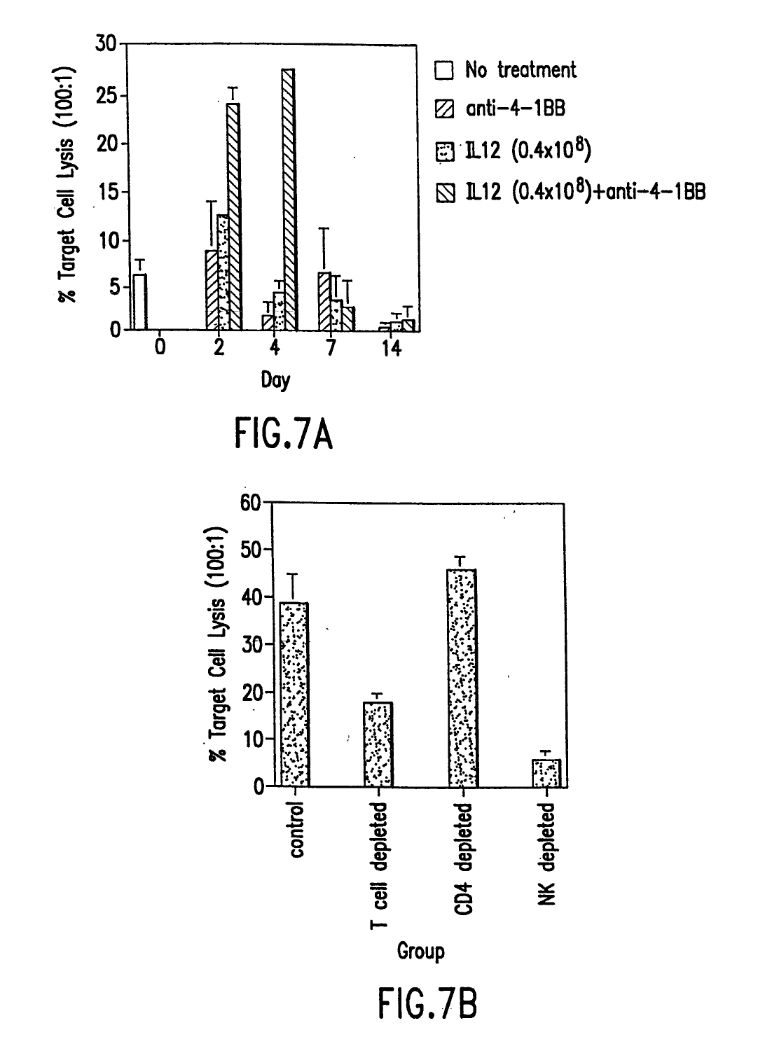

To define the synergistic action between cytokines and activation molecules, a kinetic study of direct cytolytic assay from various animal treatment group was performed (FIG. 7A).

Mononuclear cells (MNC) were isolated from the liver of the treated mice at various time points (day 0, 2, 4, 7 and 14) after gene delivery and directly assayed for their cytolytic activity against the parental MCA26 tumor cells. Adv.mIL-12 or anti-4-1BB treated animals resulted in little cytolytic activity against the parental tumor cells, which is significantly elevated in the animals after combination treatment. To identify the responsible immune cell type, the assay was repeated using MNC from animals that received the combined treatment at day 2, but after depletion of NK (DX5), or T-cell (Thy1.2), or CD4+(GK1.5) T-cell. Depletion with NK completely abolished cytolytic response, while depletion of total CD8⁺cells but not CD4+ T-cells reduced some of the cytolytic activity (FIG. 7B). The results indicate that NK cells and maybe some T-cells are involved in this synergistic tumor killing.

The Long-term Maintenance of Anti-Tumor Activity Requires Both NK and T-Cell

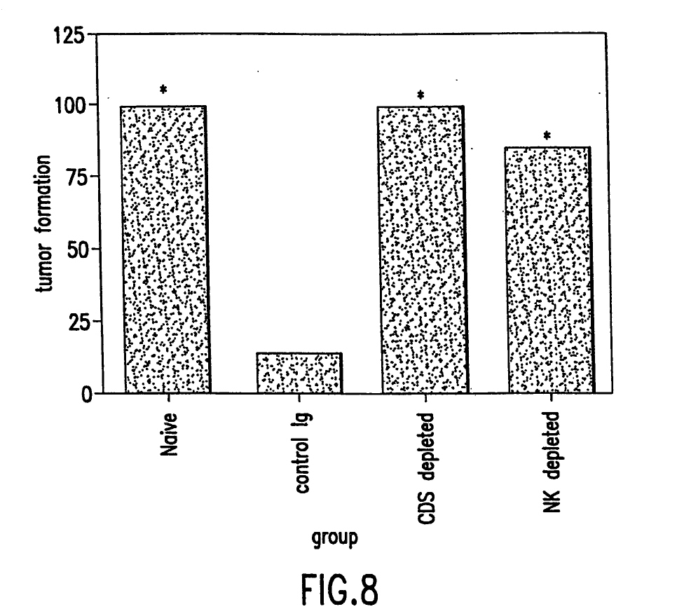

To determine which cells were responsible for the maintenance of anti-tumor activity and long-term survival of the animals after combination treatment, in vivo lymphocyte subset depletion was performed in the surviving animals prior to challenge with parental tumor cells administered at a distant site. Tumorgenic doses of parental MCA26 tumor (7×10⁴ cells) were implanted subcutaneously on the flanks of the long-term survivors and naive mice as control. The animals were observed for tumor formation over a four-week period, and the results were compared to the control Ig treated group by Fisher Exact test (FIG. 8). The challenge results showed that 100% (8/8) of the naive animals formed subcutaneous tumor, and only 14.2% (1/7) of the control Ig treated group formed tumor, suggesting that long-term anti-tumor immunity is maintained in most animals after combination treatment. In the NK or CD8+ depleted groups, 87.5% (⅞) and 100% (8/8) of the animals formed subcutaneous tumors, respectively. The results provided strong evidence that NK (p<0.0106) and CD8+ (p<0.0005) cells are maintained in the surviving animals, and both are essential in preventing the animals from tumor relapse.

6.3. Discussion

By using the combination therapy in liver tumor models and the macroscopic lung metastases tumor models, applicants have demonstrated that the long-term remission of both hepatic, breast and lung metastatic tumors with hepatic tumor gene therapy treatment. The results described herein provide a new treatment modality for cancer patients especially for those with both hepatic and multiple metastatic tumors in the other organs.

NK cells have been demonstrated to be the major and essential effector of the early anti-tumor response, and both NK and T-cells are required for the long-term tumor eradication. However, only 25% long-term survival was achieved with Adv.mIL-12 alone because only a small percentage of animals would develop long-term CTL response. The 4-1BB signals preferentially induce activated T-cell proliferation and lead to the amplification of cytotoxic T-cell response (Schuford et al, 1997, J Exp. Med. 186(1): 47-55). Applicants are the first to report the synergistic effects between 4-1BB ligand or anti-4-1BB antibody and IL-12, and bridge the early NK anti-tumor response with long-term CTL developments to achieve better therapeutic effect on both hepatic and metastatic tumors. Moreover, the combination therapy requires less IL-12, at least 10 fold less than the effective dose of IL-12 alone.

The mechanism of 4-1BB on IL-12 activated NK cells is not clearly understood. So far, in vitro cell depletion assays have indicated that NK cells and maybe some T-cells are involved in the combination treatment. The 4-1BB activated cell population that contributed to development of T helper cell and CTL development still need to be identified.

### 7. EXAMPLE

### Increased Survival Rates of Large Tumor Hepatic Metastatic Colon Carcinoma by Combination Therapy with Anti-OX40, Anti-4-1BB, and IL-12

The effectiveness of therapeutic compositions comprising a combination of anti-OX40, anti-4-1BB, and IL-12 in the long-term survival of animal models having pre-established large tumor hepatic metastatic colon cancer were evaluated according to the experimental design described below.

7.1. Materials and Methods

Recombinant Adenoviral Vectors, Mouse Model with Liver Metastases, and Therapeutic Protocol

Adv.mIL-12, a replication-defective adenoviral vector carrying the murine IL-12 genes under the transcriptional control of the Rous Sarcoma virus long terminal repeat (RSV-LTR) promoter, was constructed as demonstrated in Caruso et al., 1996, PNAS USA 93:11302-11306. MCA26 is a chemically induced colon carcinoma with low immunogenicity derived from BALB/c background (Corbett et al., 1975, Cancer Res. 35:2434-2439). Metastatic colon cancer was induced by injecting 9×10⁴ MCA26 cells into the left lateral lobe of the livers of 8 to 10-week-old female BALB/c mice (NCI) as previously described by Chen et al., 2000, Mol. Ther. 2:39-46 and Martinet et al., 2000, J. Natl. Cancer Inst. 92:931-936. At day 9 after tumor implantation, mice with liver tumors measuring 8×8 to 12×12 MM² in diameter were selected and given 6×10⁹ particles of Adv.mIL-12 or control DL312 vector (E1A-deleted control adenoviral vector) intratumorally. Following tumor implantation, mice were intraperitoneally injected with 30 μg of agonistic anti-4-1BB monoclonal antibody (Bristol-Myers Squibb, Princeton, N.J.) or control rat Ig (Accurate Chemical & Scientific Co., Westbury, N.Y.) and 100 μg of agonistic anti-OX40 monoclonal antibody (hybridoma obtained from the European Cell Culture Collection) or control rat Ig, at days 10, 12 and days 11, 13, respectively.

Cytotoxic Assay

Two types of cytotoxic assays were performed. The conventional cytotoxic lymphocyte (CTL) assay required a 5-day stimulation of splenocytes with irradiated parental tumor cells in the presence of recombinant murine IL-2 (20 U/ml). For a direct CTL assay, tumor infiltrating leukocytes (TILs) were isolated from the tumor and immediately used as the effector cells without in vitro stimulation. The CTL assay was performed as previously described. Briefly, the effector cells were incubated with [⁵¹Cr]-labeled target cells for 4 hours at 37° C. at various effector-to-target (E/T) ratios. The radioactivity released in the supernatant was measured by a gamma counter and percent specific lysis was calculated as follows: (experimental release—spontaneous release)/(maximal release—spontaneous release)×100%. To verify the effector population, effector cells were pre-incubated with anti-CD3 monoclonal antibody (10 μg/ml; Pharmingen, San Diego, Calif.) for 30 minutes at 37° C. before adding target cells.

Flow Cytometry and in vivo Depletion of CD4⁺ T-Cells

Percentage of CD4 or CD8 positive cells in a TIL population was determined by FACS analysis. Approximately 1×10⁶ viable cells were stained with F1TC conjugated anti-CD4 and PE conjugated anti-CD8 monoclonal antibodies or FITC conjugated and PE conjugated isotype control antibodies (1 μg/10⁶ cells; Pharmingen) and analyzed on a FACScan flow cytometer (Becton Dickinson, Mountain View, Calif.) with CELLQuest software. For in vivo depletion of CD4⁺ T-cells, mice were injected intraperitoneally with anti-CD4 (100 μg/mouse; Pharmingen) or control Ig two days before Adv.mIL-12 injection and every two days thereafter until termination. To confirm successful depletion, splenocytes were stained with FITC conjugated anti-CD4 antibodies and analyzed on a FACScan flow cytometer. In CD4 depleted mice, the level of CD4⁺ T-cells in the spleen is less than 0.5%.

7.2. Results & Discussion

Enhanced Primary CTL Responses in Mice Treated with Anti-OX40, Anti-4-1BB. and IL-12 Combination Therapy

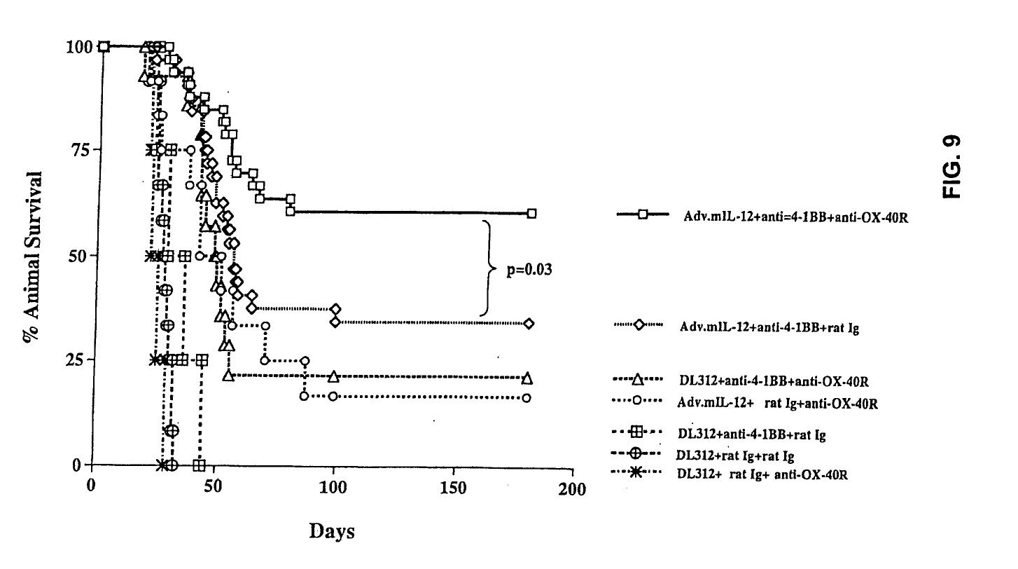

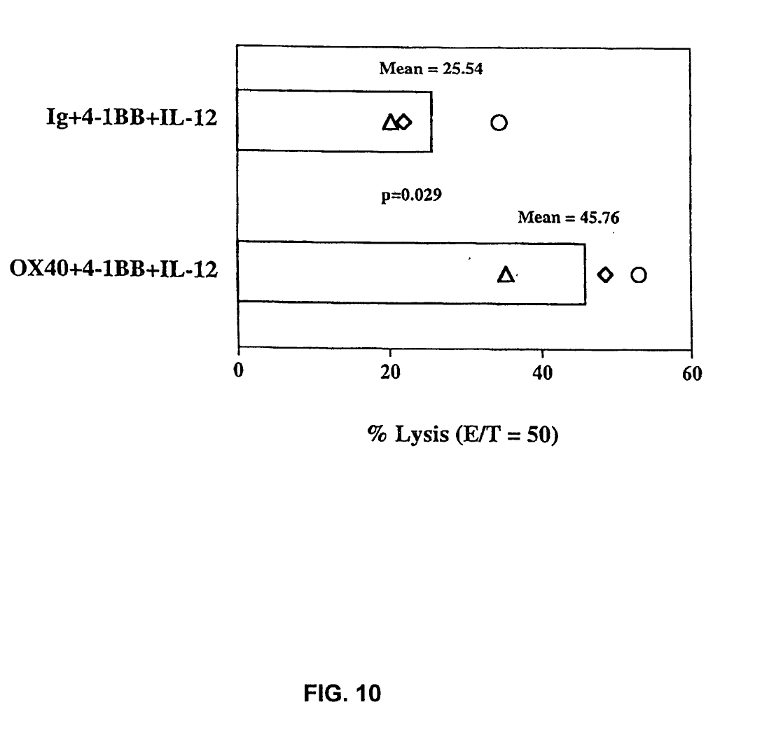

Treatment of mice bearing large tumors with sizes ranging from 8×8 to 12×12 mm² with IL-12, anti-4-1BB antibody, and anti-OX40 antibody significantly improved the survival rate of the mice relative to controls (FIG. 9). To determine whether the improved survival rate was due to an enhance anti-tumor CTL response, the ex vivo cytotoxic activity of tumor infiltrating leukocytes (TILs) was analyzed by direct CTL assay without 5 day in vitro stimulation with irradiated parental tumor cells. In the direct CTL assay, TILs were isolated from 3 mice per group at day 9 after Adv.mIL-12 injection and were directly used in the CTL assay without in vitro stimulation. TILs isolated from mice treated with IL-12, anti-4-1BB antibody, and anti-OX40 antibody exhibited higher direct CTL activity than those mice treated with IL-12 and 4-1BB (FIG. 10; 45.76% vs. 25.54% specific lysis at E/T=50, p=0.029, paired t-test). The CTL activity was completely inhibited by pre-incubation of TILs with anti-CD3 antibody, indicating that T-cells, but not NK cells, were the effector population. The results demonstrate that higher cytotoxic activity of TILs correlates with better survival rates in treated mice bearing large tumors, suggesting that OX40 ligation of CD4⁺ T-cells by agonistic antibodies can facilitate the activation and differentiation of cytotoxic T lymphocytes.

In vivo Depletion of CD4⁺ T-Cell Tests Demonstrate Increase in Tumor Infiltrating CD8⁺ T-Cells

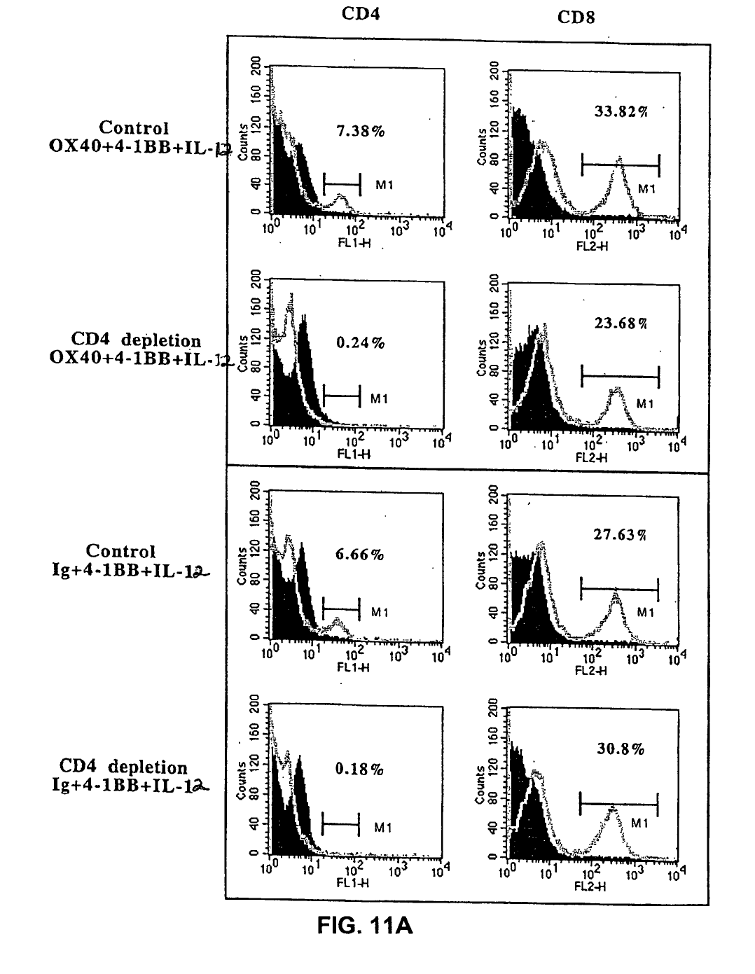

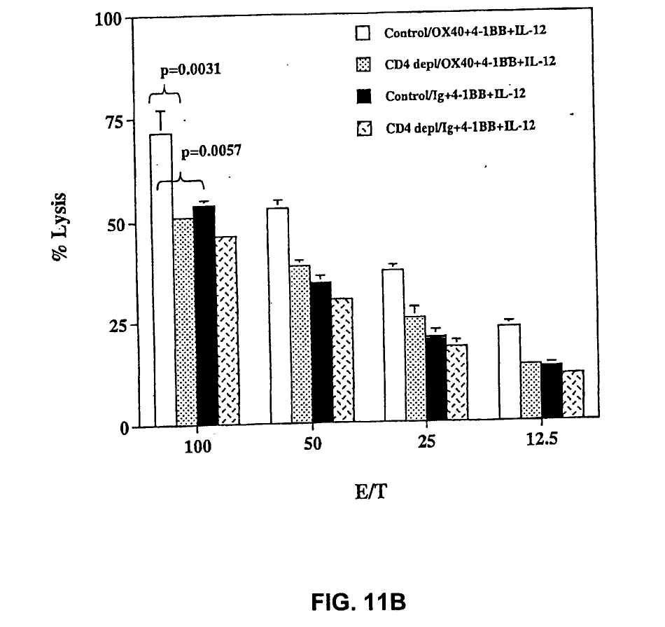

To assess the contribution of CD4⁺ T-cells to the higher TIL direct CTL activity, experiments with in vivo depletion of CD4⁺ T-cells were performed. Nine days after Adv.mIL-12 injection, TILs were isolated from 4 mice per group and analyzed for CD4, CD8 markers and ex vivo cytotoxic activity. In IL-12 and anti-4-1BB antibody treated mice, CD4⁺ and CD8⁺ T-cells constituted 6.66% and 27.63% of the TIL population respectively (FIG. 11A). In mice treated with the IL-12, anti-4-1BB antibody, and anti-OX40 antibody combination therapy, a slight increase in CD4⁺ T-cells (7.38% vs. 6.66%) and a substantial increase in CD8⁺ T-cells (33.82% vs. 27.63%) were observed. Treatment with anti-OX40 agonistic antibody did not alter CD4 and CD8 representation in the spleen. Interestingly, there was a significant decrease in the number of CD8⁺ T-cells (23.68% vs. 33.82%) in TILs isolated from in vivo CD4-depleted mice with the IL-12, anti-4-1BB antibody, and anti-OX40 antibody combination therapy while no substantial change of CD8⁺ T-cells in TILs was observed in CD4-depleted mice receiving IL-12 and anti-4-1BB antibody treatment. The CD8⁺ population in the spleen was not affected by in vivo CD4 depletion in mice receiving IL-12, anti-4-1BB antibody or IL-12, anti-4-1BB antibody, and anti-OX40 antibody therapy. The results suggest that OX40 ligation by agonistic antibody can facilitate the recruitment and/or expansion of tumor infiltrating CD8⁺ T-cells while exerting no significant effect on splenic CD4⁺ or CD8⁺ T-cells.

The ex vivo CTL activity of TILs was examined to assess the effect of CD4⁺ T-cell activation by anti-OX40 antibody on the development of the CTL response against tumor cells. The direct CTL activity of TILs isolated from mice treated with the IL-12, anti-4-1BB antibody and anti-OX40 antibody combination therapy was significantly enhanced when compared to that from IL-12 and anti-4-1BB antibody treated mice (FIG. 11B; 71.63% vs. 53.93% specific lysis at E/T=100, p=0.0057, at all E/T ratios tested, p<0.01). More importantly, in vivo depletion of CD4⁺ T-cells significantly reduced TIL direct CTL activity in mice treated with the IL-12, anti-4-1BB antibody and anti-OX40 antibody combination therapy (71.63% vs. 51.2% specific lysis at E/T=100, p=0.0031, at all E/T ratios test, p<0.01, t-test) but not in IL-12 and anti-4-1BB antibody treated mice. With in vitro CD4 depletion, the ex vivo TIL CTL activity of mice treated with the IL-12, anti-4-1BB antibody and anti-OX40 antibody combination therapy was reduced to a level similar to that of IL-12 and anti-4-1BB antibody treated mice (51.2% vs. 53.93% specific lysis), suggesting that the enhanced TIL cytotoxic activity observed in mice treated with the IL-12, anti-4-1BB antibody and anti-OX40 antibody combination therapy is a direct result of OX40 co-stimulation of CD4⁺ T-cells.

Treatment of Mice with Advanced Liver Metastases of Colon Carcinoma by the Combination of IL-12 Gene Therapy and 4-1BB Plus OX40 Co-stimulation

Liver is the major organ of metastases for most human gastrointestinal cancers. Therefore, a murine hepatic colon metastases model generated by direct intrahepatic implantation of a lowly immunogenic colon carcinoma MCA26 was used to assess the potential for coordinated activation of CD4⁺, CD8⁺ T-cells, and NK cells by OX40 and 4-1BB co-stimulation and IL-12 combination therapy for treating mice with large tumors. BALB/c mice bearing syngeneic tumors measuring between 8×8 to 12×12 mm² in diameter were chosen and directly injected with Adv.mIL-12 or the control vector DL312. Mice were given intraperitoneal injections of anti-4-1BB antibody, anti-OX40 antibody, or anti-4-1BB antibody and anti-OX40 antibody, or control Ig alone. As shown in FIG. 9, 60% of mice treated with IL-12, anti-4-1BB antibody and anti-OX40 antibody were alive and tumor-free after 180 days, while only a 34% survival rate was observed in mice treated with IL-12 and anti-4-1BB antibody (p=0.03, log-rank test). Treatment with anti-4-1BB antibody and anti-OX40 antibody, or IL-12 and anti-OX40 antibody resulted in 21% and 16% long-term survival rate, respectively. Mice treated with rat Ig and control vector or either antibody (4-1BB or OX40) and control vector all died within 50 days after tumor inoculation. The results show that combination therapy with 4-1BB, OX40 co-stimulation and IL-12 significantly improves therapeutic efficacy in a large tumor setting. Moreover, all three reagents are essential to the treatment of large tumors because a therapy lacking any of the three greatly compromised the therapeutic efficacy.

Enhanced Memory CTL Responses in Mice Cured by the Anti-OX40, Anti-4-1BB, and IL-12 Combination Therapy

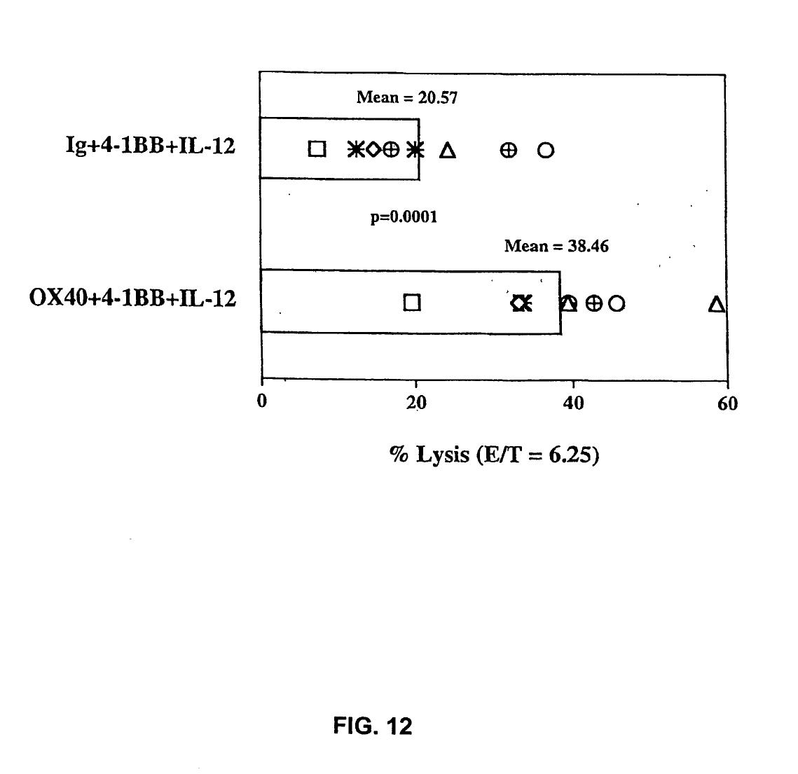

In the IL-12 and anti-4-1BB antibody combination treatment, CD8⁺ T-cells are involved in the initial anti-tumor immune response and long-term protective immunity against MCA26 tumors. Although CD4⁺ T-cells are not required for long-term protective immunity against parental tumor cells, the in vivo depletion of CD4⁺ T-cells before treatment with Adv.mIL-12 and anti-4-1BB antibody resulted in a decrease in long-term survival of treated mice, suggesting that CD4⁺ T-cells are essential for the development of persistent anti-tumor activity. To further address whether the activation of CD4⁺ T-cells can facilitate the development of memory CTL responses against MCA26 tumor cells, the cytolytic activity of splenocytes isolated from cured long-term surviving mice followed by 5 day in vitro stimulation with irradiated MCA26 cells was analyzed. As shown in FIG. 12, splenocytes isolated from mice treated with the IL-12, anti4-1BB antibody and anti-OX40 antibody combination therapy exhibited significantly higher cytolytic activity against MCA26 tumor cells when compared to those from IL-12 and anti-4-1BB antibody treated mice (38.4% vs. 20.5% at E/T=6.25, p=0.0001, paired t-test). The tumor lysis was completely inhibited by pre-incubating effector cells with anti-CD3 antibody, suggesting that the cytolytic activity was mediated by cytotoxic T-cells. The results indicate that the activation of CD4+T cells by anti-OX40 antibody in conjunction with the IL-12 and anti-4-1BB antibody therapy can induce a significantly higher memory CTL response against MCA26 tumor cells in cured, tumor-free mice.

Taken together, the experiments presented here demonstrate that coordinated activation of NK, CD8⁺, and CD4⁺ T-cells with the combination of Adv.mIL-12, anti-4-1BB antibody, and anti-OX40 antibody can significantly enhance therapeutic efficacy when treating large tumors (8×8 to 12×12 mm²). Recently, it was reported that the engagement of OX40 in vivo alone by OX40L-Ig fusion proteins or agonistic anti-OX40 monoclonal antibodies substantially enhanced tumor free survival of treated mice in some tumor models but not in others (Weinberg et al., 2000, J. Immunol. 164:2160-2169 and Kjaergaard et al., 2000, Cancer Res. 60:5514-5521). The therapeutic efficacy of anti-OX40 antibody depends on the tumor immunogenicity as well as the anatomic site of tumor growth (Kjaergaard et al., 2000, Cancer Res. 60:5514-5521). In the mouse model utilized herein, anti-OX40 antibody alone did not improve the survival rate of treated mice bearing large tumors. Only in conjunction with the Adv.mIL-12 and anti-4-1BB antibody therapy, which has been shown to activate NK and CD8⁺ T cells (see Section 6), can anti-OX40 antibody improve therapeutic efficacy. The activation of CD4⁺ T-cells through anti-OX40 antibody significantly enhances both the primary and persistent memory CTL responses in mice treated with the Adv.mIL-12, anti-4-1BB antibody and anti-OX40 antibody combination therapy. Without intending to limited to a particular mechanism, the mechanism underlying the improved therapeutic efficacy of the Adv.mIL-12 and anti-4-1BB antibody treatment by anti-OX40 antibody co-stimulation in a larget tumor setting is, at least in part, through enhancing primary clonal expansion and memory T-cell survival as well as enabling CD8⁺ T-cells to traffic to and/or remain within the tumor, thereby facilitating tumor killing (Marzo et al., 2000, J. Immunol. 165:6047-6055). Although in vivo depletion of CD8⁺ T-cells before treatment indicates that the CD8⁺ T-cell is the main effector cell for tumor eradication in the Adv.mIL-12, anti-4-1BB antibody and anti-OX40 antibody combination, the direct involvement of anti-OX40 antibody activated CD4⁺ T-cells in the effector phase of tumor rejection through cytokine activated eosinophils and macrophages cannot be excluded.

In summary, ligation of OX40 in vivo with agonistic antibodies significantly improves the therapeutic efficacy of the Adv.mIL-12 and anti-4-1BB antibody therapy for treating large tumors (8×8 to 12×12 mm²) in a murine hepatic metastatic colon carcinoma model. Furthermore, OX40⁺ T-cells have been reported in several human malignancies (Vetto et al., 1997, Am. J. Surg. 174:258-265; Ramstad et al., 2000, Am. J. Surg. 179:400-406), underscoring the significance of OX40 co-stimulation for cancer therapy. The combination of Adv.mIL-12, anti-4-1BB antibody and anti-OX40 antibody to coordinately activate NK, CD8⁺, and CD4⁺ T-cells may provide a new treatment modality for patients with metastatic colon cancer.

### 9. EXAMPLE

### Immunosuppressive Effect of Myeloid Progenitor Cells Accumulating in the Spleen and Bone Marrow of Animals Bearing Tumors

Tumor Growth Induces the Accumulation of Gr-1⁺/CD11b⁺ Cells in the Spleen and Bone Marrow

Flow cytometry was used to analyze the frequency of Gr-1⁺ (Ly-6G⁺) and Mac-1⁺ (CD11b⁺) cells in the spleen and bone marrow of mice bearing MCA-26 (colon) or JC (breast) tumors. Freshly derived spleen or bone marrow cells depleted of erythrocytes were used for staining. While only 2.8±0.3% of the spleen cells from tumor-free mice (naive) expressed both Gr-I and Mac-I antigens, 43.8±2.5% and 13.3±7.5% of the spleen cells from colon tumor-bearing mice and breast tumor-bearing, respectively, expressed both Gr-1 and Mac-1 antigens (Table 1). The percentage of bone marrow cells expressing both Gr-1 and Mac-1 antigens from colon tumor-bearing mice (79.3±1.3%) and breast tumor-bearing mice (80.2±4.5%) were double the percentage of bone marrow cells expressing both Gr-1 and Mac-1 antigens from tumor-free mice (34.5±3.8%). See Table 1. These results indicate that tumor-bearing mice have a greater number of Gr-1⁺/Mac-1⁺ cells in their spleens and bone marrow than tumor-free mice.

TABLE 1
Increase of Gr-1⁺/Mac-1⁺Cells in the Spleens and
Bone Marrow (BM) of MCA26 and JC Tumor-Bearing Mice

| | normal mice | | colon tumor mice | | breast tumor mice | |
|---|---|---|---|---|---|---|
| Marker | spleen % | BM % | spleen % | BM % | spleen % | BM % |
| Gr-1 | 13.2 ± 1.2 | 41.7 ± 6.3 | 48.3 ± 2.4 | 80.9 ± 5.3 | 52.8 ± 14.8 | 88.2 ± 3.0 |
| Mac-1 | 6.6 ± 0.8 | 48.1 ± 11.7 | 45.8 ± 2.6 | 90.1 ± 1.9 | 50.4 ± 3.0 | 88.7 ± 2.3 |
| Gr-1/Mac-1 | 2.8 ± 0.3 | 34.5 ± 3.8 | 43.8 ± 2.5 | 79.3 ± 1.3 | 13.3 ± 7.5 | 80.2 ± 4.5 |

The results shown are representative of three separate experiments.

A Fractionated Myeloid Progenitor Cell Population Inhibits CD3/CD8 Induced T-Cell Proliferation

Myeloid progenitor cell populations from bone marrow or spleen cell suspensions from tumor-free or MCA26 tumor-bearing mice were depleted of plastic adherent cells overnight and fractionated according to their density using Percoll gradient. Two fractionated populations of myeloid progenitor cells were assessed for their ability to inhibit CD3 or CD3/CD28 induced proliferation of naive splenic T-cells. Splenocytes from tumor-free mice (naive splenocytes) were co-cultured for 72 hours, in the presence of anti-CD3 monoclonal antibody or both anti-CD3 monoclonal antibody and anti-CD28 monoclonal antibody, with fraction II (Fr. II; 50-60%, 1.063-1.075 g/ml) cells or fraction III (Fr.III; 60-70%, 1.075-1.090 g/ml) cells derived from the bone marrow of tumor-free mice, the bone marrow of MCA26 tumor-bearing mice, or the spleen of MCA26 tumor-bearing mice. Proliferation of splenic T-cells was measured by ³H-thymidine incorporation.

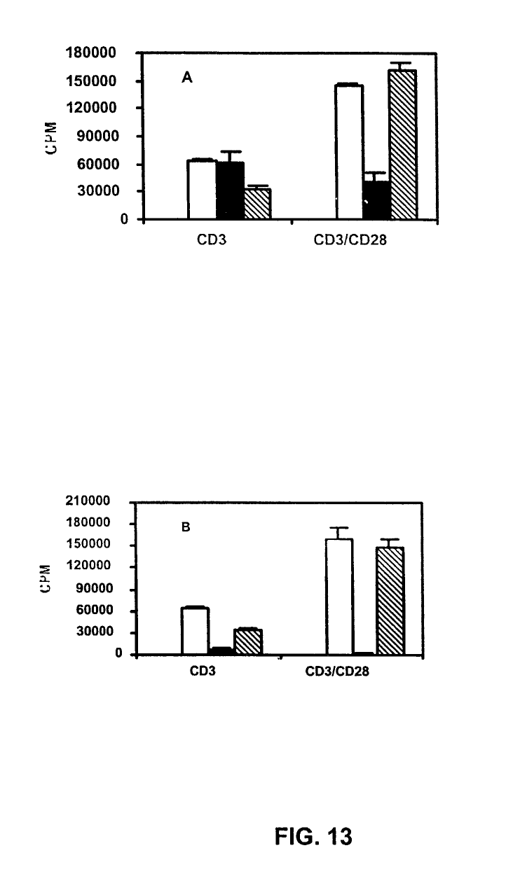

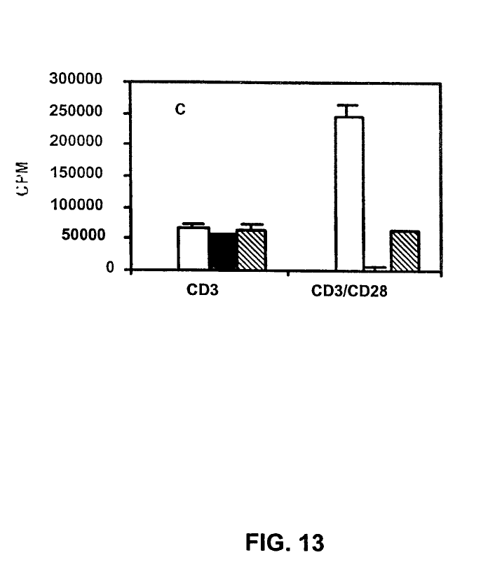

As shown in FIGS. 13A-13C, a reduction in anti-CD3 monoclonal antibody induced proliferation of splenocytes was detected when the splenocytes were co-cultured with Fr. II cells derived from the bone marrow or spleen of MCA26 tumor-bearing mice than when the splenocytes were co-cultured with Fr. II cells derived from the bone marrow of tumor-free mice. Little to no change in anti-CD3 monoclonal antibody induced proliferation of splenocytes was detected when the splenocytes were co-cultured with Fr. III cells derived from the bone marrow of tumor-free mice, or the bone marrow or spleen of MCA26 tumor-bearing mice. Thus, Fr.II cells derived from the bone marrow or spleen of MCA26 tumor-bearing mice reduce the anti-CD3 monoclonal antibody induced proliferative response of naive splenocytes.

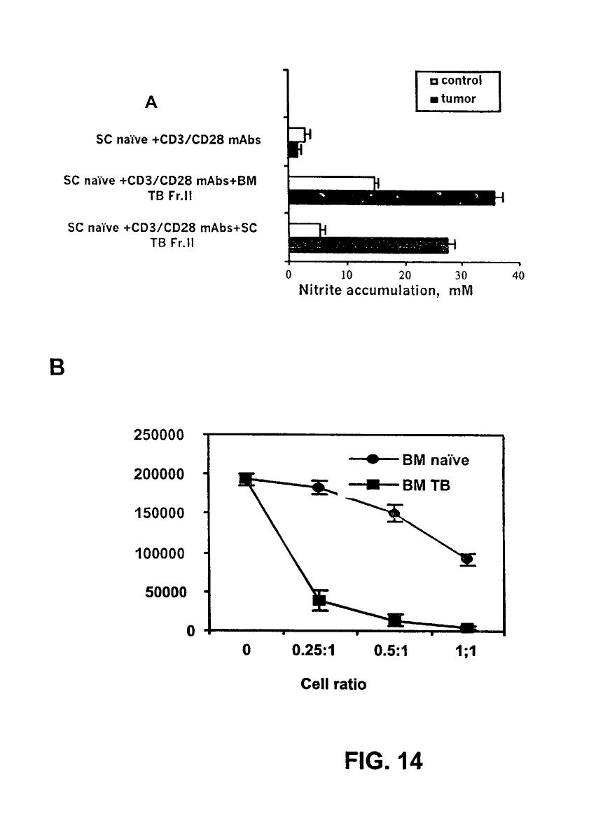

As shown in FIGS. 13A-13C, greater than 90% of the proliferative response induced anti-CD3 and anti-CD28 monoclonal antibodies was inhibited when naive splenocytes were co-cultured with Fr. II cells derived from the bone marrow or spleen of MCA26 tumor-bearing mice, as compared to when naive splenocytes were cultured alone. A significant reduction in anti-CD3 and anti-CD28 monoclonal antibody induced proliferation was detected when naive splenocytes were co-cultured with Fr. II cells derived from the bone marrow of MCA26 tumor-bearing mice, as compared to when naive splenocytes were co-cultured with Fr.II cells derived from the bone of tumor-free mice (FIGS. 13A-13B and FIG. 14). Similarly, a significant reduction in anti-CD3 and anti-CD28 monoclonal antibody induced proliferation was detected when naive splenocytes were co-cultured with Fr. II cells derived from the spleen of MCA26 tumor-bearing mice, as compared to when naive splenocytes were co-cultured with Fr.II cells derived from the spleen of tumor-free mice. Thus, Fr.II cells derived from the bone marrow or spleen of MCA26 tumor-bearing mice significantly reduce the anti-CD3 and anti-CD28 monoclonal antibody induced proliferative response of naive splenocytes.

As shown in FIGS. 13A-13B, no reduction in anti-CD3 and anti-CD28 monoclonal antibody induced proliferation was detected when naive splenocytes were co-cultured with Fr.III cells derived from the bone marrow of MCA26 tumor-bearing mice, as compared to when naive splenocytes were cultured alone or co-cultured with Fr.III cells derived from the bone marrow of tumor-free mice. Although a reduction in anti-CD3 and anti-CD28 monoclonal antibody induced proliferation was detected when naive splenocytes were co-cultured with Fr.III cells derived from the spleen of MCA26 tumor-bearing mice as to compared to when naive splenocytes were cultured alone, the effect of Fr.III cells derived from the spleen of tumor-free mice was not assessed. Thus, the effect on Fr.III cells derived from the spleen of MCA26 tumor-bearing mice on anti-CD3 and anti-CD28 monoclonal antibody induced proliferation of naive splenocytes is unclear.

In addition to the CD3/CD28 induced proliferation assay, a MLR (Mixed lymphocyte reaction) and tumor specific cytolytic T-cell assay were performed in the presence of the immune suppressive myeloid cells derived from bone marrow of MCA-26 tumor-bearing mice. The proliferative and cytolytic responses were inhibited in the presence of Fr. II cells derived from the bone marrow of tumor-bearing mice. The proliferative and cytolytic responses were inhibited less in the presence of other fractions of cells (e.g., Fr. III) than the inhibition detected in the presence of Fr.II cells derived from the bone marrow of tumor-bearing mice. These results suggest that Fr.II cells derived from the bone marrow or spleen of tumor-bearing mice have an immunosuppressive effect.

Gr-1⁺ Cells in Fr.II Contribute to the Immunosuppressive Effect of Fr.II

To assess the contribution of Gr-1⁺/CD11b⁺ cells to immune suppression, Gr-1⁺ cells in Fr. II were depleted using a panning technique. The depleted non-adherent (post-panning) cell population was tested for its ability to inhibit CD3/CD28 stimulated T-cell proliferation. Splenocytes from tumor-free mice were co-cultured for 72 hours, in the presence of anti-CD3 and anti-CD28 monoclonal antibodies, with Gr-1⁺-depleted fraction II cells derived from the bone marrow of tumor-free mice or the bone marrow of MCA26 tumor-bearing mice. Proliferation of splenic T-cells was measured by ³H-thymidine incorporation. No significant difference in anti-CD3 and anti-CD28 monoclonal antibody induced proliferation was detected when naive splenocytes were co-cultured with Gr-1⁺-depleted fraction II cells derived from the bone marrow of MCA26 tumor-bearing mice than when naive splenocytes were co-cultured with Gr-1⁺-depleted fraction II cells derived from the bone marrow of tumor-bearing mice. The results demonstrate that the T-cell response can be restored by depletion of Gr-1⁺ cells. Thus, it appears that Gr-1⁺ cells are responsible for inhibition of CD3/CD28-stimulated T cell proliferation in the presence of the inhibitory myeloid progenitors in fraction II derived from BM or spleen of MCA-26 tumor bearers.

Immune Suppression Mediated by Gr-1⁺ Cells is Reversed by Using a Combination of Peroxynitrite and Nitric Oxide Inhibitors

Nitric oxide (NO) is known as the major mediator of natural suppressor activity.

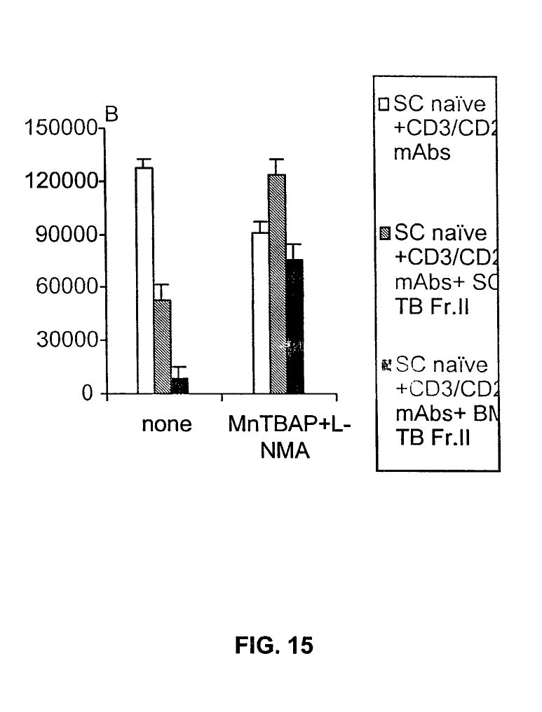

NO production may result in the induction of apoptosis and suppression of cell growth. The level of nitrites in co-cultures of fractionated tumor-bearer-derived bone marrow or spleen cells and naive spleen cells stimulated with anti-CD3 and anti-CD28 monoclonal antibodies was measured. In a representative experiment (FIG. 15A), high nitrite levels were detected in the culture supernatants of stimulated spleen cells co-cultured with Fr.II cells, i.e., cultures exhibiting a high level of suppression. Fr.II cells cultured alone did not generate nitric oxide.

To determine the involvement of nitrites in the immune suppression caused by Fr.II cells, L-NMMA and MnTBAP, competitive inhibitors of inducible NO synthase (iNOS) and superoxide dismutase (SOD), respectively, were added to the cultures containing anti-CD3 and anti-CD28 monoclonal antibodies, naive splenocytes, and Fr.II cells derived from the spleen or bone marrow of tumor-bearing mice or tumor-free mice. Proliferation of the splenocytes was assessed by ³H-thymidine incorporation. As shown in FIG. 15B, the addition of L-NMMA and MnTBAP to the cultures almost completely reversed the immune suppression mediated by Gr-1⁺ cells.

Fr.II Myeloid Progenitor Cells Inhibit the Proliferative Response of HA-TCR Transgenic T-cells Induced by HA Peptide

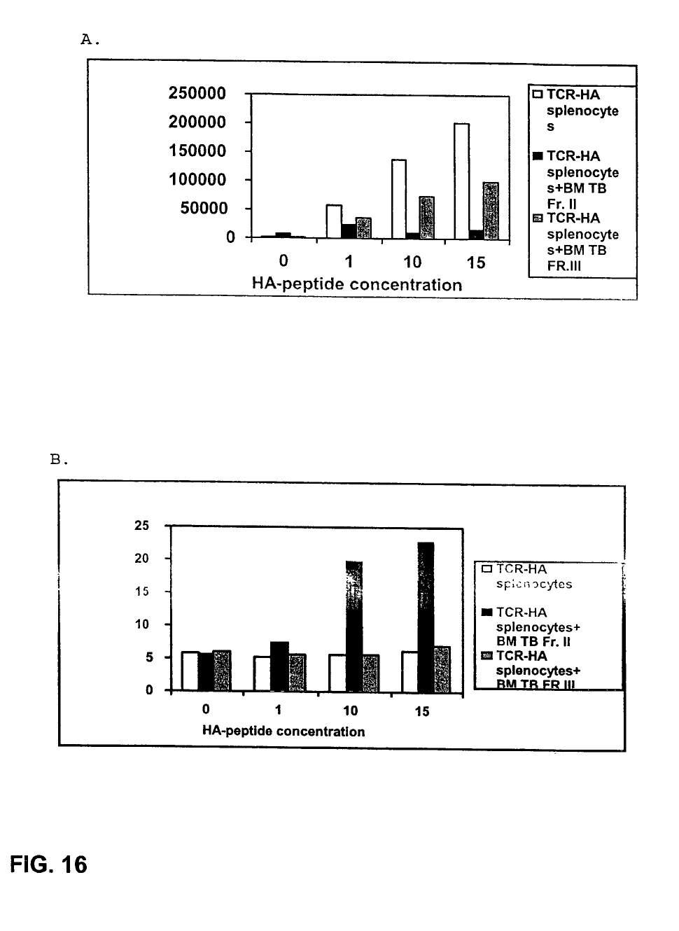

The effect of Fr.II myeloid progenitor cells or tumor infiltrating leukocytes (TILs) on HA specific T-cell proliferation was assessed by ³H-thymidine incorporation. Splenocytes from tumor-free HA TCR transgenic mice were co-cultured for 72 hours, in the presence of CD4 HA peptide, with Fr.II or Fr.III cells derived from the bone marrow of MCA26 tumor-bearing mice. Proliferation of splenic T-cells was measured by ³H-thymidine incorporation. As shown in FIG. 16A, the myeloid progenitor cells from bone marrow Fr.II and TILs can significantly inhibit CD4 HA peptide mediated T cell proliferation, but this effect is less pronounced for cells isolated from Fr.III. Interestingly, the level of NO production from the Fr.II myeloid progenitor cells is significantly higher than observed with regular HA peptide mediated splenocyte proliferation or with HA splenocytes co-cultured with cells from Fr.III. Nitrite accumulation is correlated with T cell activation using higher concentrations of HA peptide (FIG. 16B).

The Immunophenotype of Bone Marrow Fr.II Myeloid Progenitor Cells Isolated from Tumor Bearing vs. Naive Animals

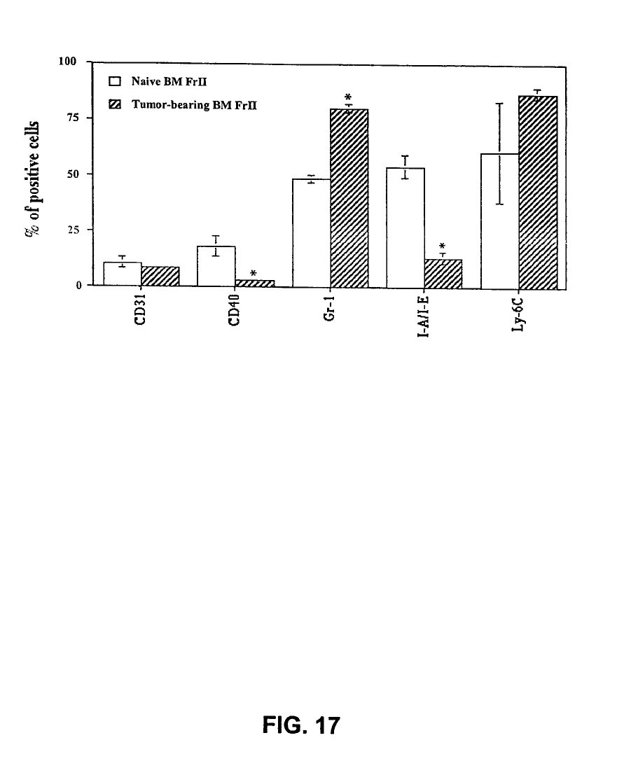

The immunophenotype of Fr.II cells derived from the bone marrow of tumor-free mice (naive mice) and the bone marrow of MCA26 large tumor-bearing mice was assessed by flow cytometric analysis (FIG. 17). Preliminary results indicate that significant higher numbers of Gr-1⁺ cells (80.34%) and Ly-6c⁺ cells (83.52%) were found in the Fr.II cells derived from the bone marrow of tumor-bearing mice than in the Fr.II cells derived from the bone marrow of naive mice (48.7% for Gr-1⁺ and 60.8% for Ly-6c⁺). Interestingly, the MHC Class II and CD40 expression are significant lower (13.4% and 3.3%) in the Fr.II cells derived from the bone marrow of tumor-bearing mice as compared to the Fr.II cells derived from the bone marrow of naive mice (54.3% and 18.2%). These results suggest that the Fr.II myeloid progenitor cell population in tumor-bearing mice is a poorly differentiated myeloid precursor, which is accumulated and induced by a large tumor burden.

Depletion of F4/80 Positive Bone Marrow Fr.II Cells can Significantly Block the Inhibitorv Effect of Myeloid Progenitor Cells

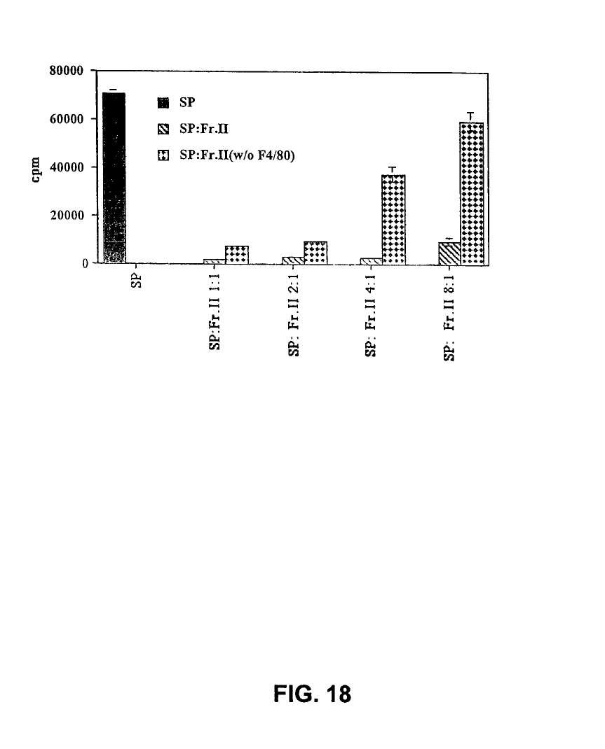

In order to further assess which cell type was involved in the myeloid progenitor cell mediated inhibition, myeloid progenitor Fr.II cells derived from the bone marrow of JC tumor-bearing mice were tested for their effect on HA peptide mediated CD8 TCR T-cell proliferation. Splenocytes from tumor-free HA-TCR transgenic mice were co-cultured for 72 hours, in the presence of CD8 HA peptide, with Fr.II or Fr.II cells-depleted of F4/80 cells derived from the bone marrow of JC (breast) tumor-bearing mice. Proliferation of splenic T-cells was measured by ³H-thymidine incorporation. Fr.II significantly inhibited CD8 HA peptide mediated T cell proliferation (FIG. 18). Interestingly, depletion of the F4/80⁺ cells from the Fr.II cells significantly blocked the inhibition of proliferation at the 4:1 and 8:1 ratio. These results indicate that the F4/80⁺ myeloid progenitor cells are involved in this inhibitory effect of Fr.II cells.

### 10. EXAMPLE

### Differentiation of Myeloid Progenitor Cells

Differentiation of Bone Marrow Fr.II Myeloid Progenitor Cells into Dendritic Cells in vitro

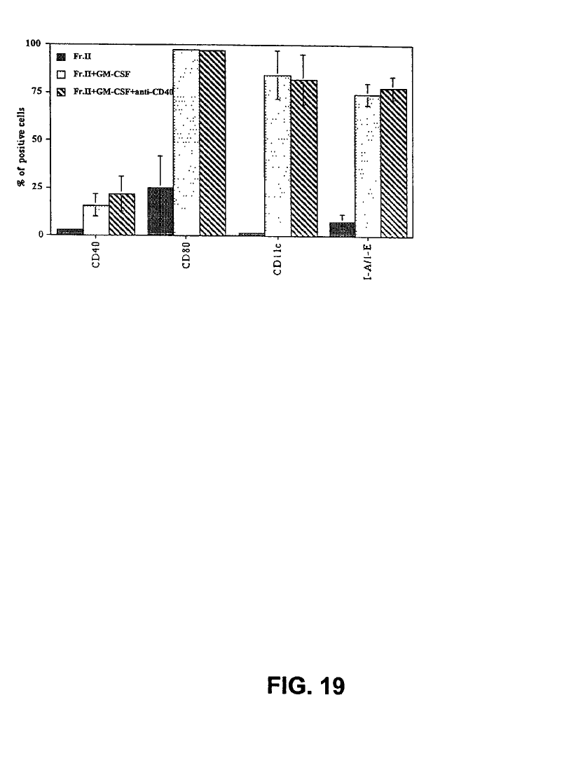

The ability of Fr.II cells derived from the bone marrow of MCA26 tumor-bearing mice to differentiate into dendritic cells when cultured under various conditions was assessed. Fr.II cells from derived the bone marrow of tumor-free or MCA26 tumor-bearing mice were cultured for 10 days with GM-CSF. Then non-adherent cells were harvested and cultured for additional 24 hours in the presence of GM-CSF or GM-CSF and anti-CD40 monoclonal antibody. The non-adherent cells were stained and analyzed for the expression of various cell surface molecules by flow cytometry. The expression of CD40, CD80, and MHC class II was relatively low in the tumor-bearing mice. However, after culturing the Fr. II cells with GM-CSF or GM-CSF and anti-CD40 monoclonal antibody, the expression of CD40, CD 80, CD11c and MHC class II by the Fr. II cells was significantly increased (FIG. 19). The expression levels of CD40, CD 80, CD11c and MHC class II were even higher than those of the cultured Fr. II cells derived from the bone marrow of naive mice.

In vitro GM-CSF Stimulation Promotes the Differentiation of Myeloid Progenitor Cells in the TIL into Dendritic Cells and Macrophages

The ability of in vitro stimulation with GM-CSF to promote the differentiation of tumor infiltrating lymphocytes (TILs) into dendritic cells or macrophages by was assessed. The TILs were isolated from large tumor-bearing mice, separated on a lymphocyte separation gradient, cultured with or without GM-CSF (100 ng/ml) for 5 days, and then stained with various cell surface markers. A reproducibly high percentage of Gr-1⁺ cells was resent in the TIL population (65.5%), while the percentage of dendritic cells in the TIL population was very low (0.31%); some macrophages (14.9%) were also present in the TIL population. In the presence of GM-CSF, the myeloid progenitor cells in the TIL population proliferated and differentiated into dendritic cells (13.9%) and macrophages (24.3%). The number of Gr-1⁺ cells detected in the TIL population following GM-CSF stimulation was significantly reduced (42.7%). These results suggest that the Gr-1⁺ progenitor cells in the TILs will proliferate and differentiate into dendritic cells and macrophages in the presence of the proper cytokine stimulation.

Differentiation of Myeloid Progenitor Cells into Dendritic Cells and Recruitment to the Tumor Site in the Presence of GM-CSF Stimulation in vivo

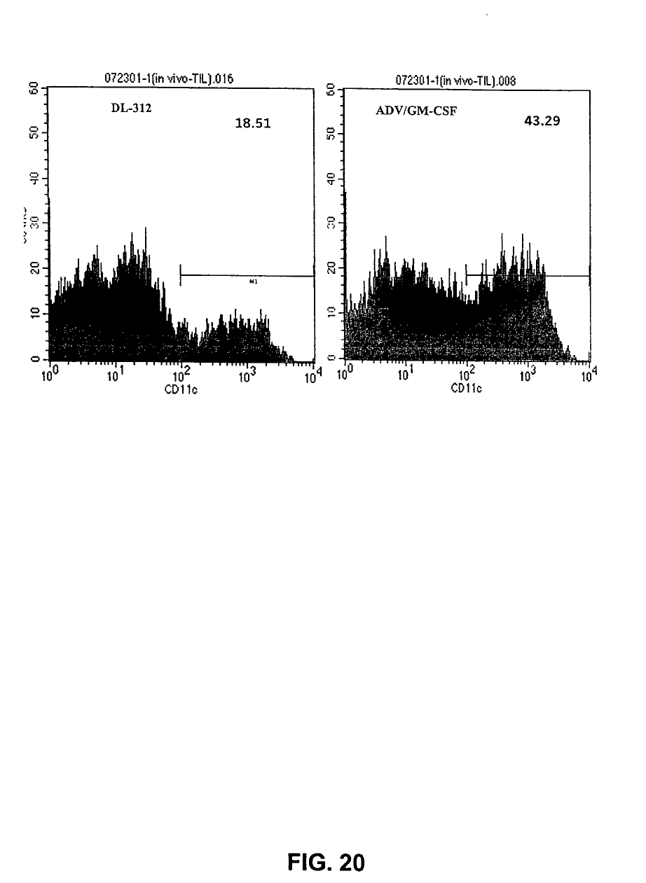

The effect of the administration of recombinant adenovirus expressing GM-CSF (ADV/mGM-CSF) to MCA26 tumor-bearing mice on the myeloid progenitor cell population in tumor infiltrating lymphocytes (TILs) was assessed. MCA26 tumor-bearing mice were intratumorally injected with ADV/mGM-CSF (4.4×10⁹ pfu/mouse) or control vector (DL-312) and seven days later the TILs were isolated and stained for Gr-1, Ly-6c and CD11c. The stained cells were analyzed by flow cytometry. As shown in FIG. 20A, the intratumoral administration of ADV/mGM-CSF to the mice significantly increased the number dendritic cells in the TIL population. The percentage of CD11c⁺ cells in ADV/mGM-CSF treated mice (n=6) increased significantly when compared to the mice treated with control vector (n=6)(40.7±4.0 vs. 20.7±3.0, p<0.05). See FIG. 20B.

Adoptive-Transferred CFSE Labeled Fr.II Cells from ADV/mGM-CSF Treated Mice Promote the Differentiation of Myeloid Progenitor Cells into Mature Dendritic Cells

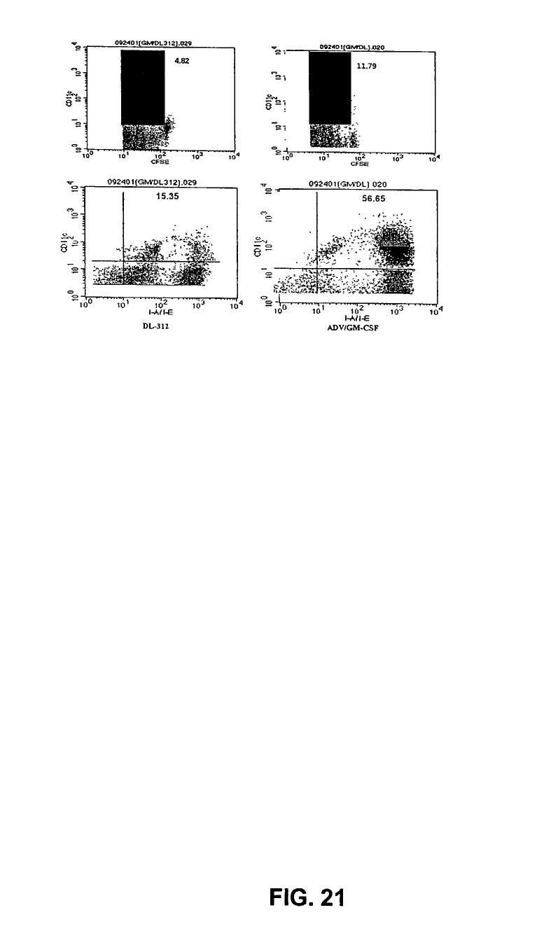

The ability of CFSE labeled Fr.II cells from intratumorally injected ADV/mGM-CSF treated MCA26 tumor-bearing mice infused into the tail of tumor-bearing mice to promote the differentiation of myeloid progenitor cells into more mature dendritic cells. MCA-26 tumor bearing mouse received 4.4×10⁹pfu/mouse of ADV/mGM-CSF (FIGS. 21B and 21D) or DL312, control vector (FIGS. 21A and 21C) by intratumor injection. Twenty-four hours later, bone marrow and spleen Fr.II cells were labeled with CFSE (10 μM) and adoptive transferred to recipients. Each recipient mouse received 2×10⁷ CFSE-labeled Fr.II cells by tail vein infusion. Splenocytes were isolated 5 days after adoptive transfer and stained with PE-CD11c and biotinylated-MHC II (I-A/I-E) or isotype controls. The expression of CD11c and MHC II by splenocytes was analyzed by flow cytometry. The results indicate that the myeloid progenitor cells underwent multiple cycles of proliferation. As shown in FIGS. 21A-21D, a significant increase in CD1 Ic and MHC Class II double positive cells was observed in GM-CSF treated mice (44.9±1.0%) and a less significant increase in CD11c and MHC Class II double positive cells was observed in control vector treated mice (19.9±2.7%, p<0.01).

### 11. EXAMPLE

### The Eradication of Tumors in Mice Treated by ADV/GM-CSF in Conjunction with IL-12 and Anti-4-1BB Immune Activation

The therapeutic effect of the administration of ADV/GM-CSF, anti-4-1BB, and IL-12 on the long-term survival of animal models having pre-established tumor hepatic metastatic colon cancer was evaluated. Metastatic colon cancer was induced by implanting 7×10⁴ MCA26 cells into the left lobe of the liver of 8-10 week old female BALB/c mice (Taconic). At day 7, mice bearing 5×6 mm² size tumors were injected with ADV/mGM-CSF (n=10) or control DL-312 virus (n=10) or buffer alone (n=10). Tumors were allowed to grow for 8 days. Eight days after the GM-CSF injection, the animals with tumor sizes larger than 10 mm² were subjected to treatment with the ADV/IL-12 virus and anti-4-1BB monoclonal antibody. Adv.mIL-12 or control DL312 vector were injected intratumorally in a 50 μl volume of 10 mM Tris-HCl (pH 7.4)/1 mM MgCl₂/10% (vol/vol) glycerol/Polybrene (20 μg/ml) and 50pg anti-4-1BB monoclonal antibody or control rat Ig was administered intraperitoneally (i.p.). Long term survival was then recorded.

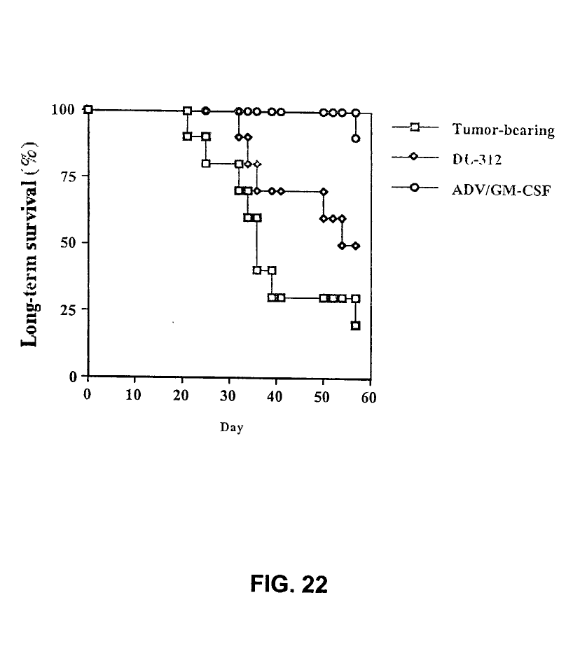

All of the animals pre-injected with ADV/mGM-CSF virus and subsequently treated with IL-12 virus and anti4-1BB monoclonal antibody had significantly prolonged survival and seven of the ten animals exhibited not only eradication of tumors, but remained tumor free in the long-term (FIG. 22). The results indicate that the maturation of myeloid progenitor cells is important for T-cell activation by IL-12 and 4-1BB.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All patent and non-patent publications cited herein are incorporated by reference in their entirety.

What is claimed is:

1. A method for treating cancer, an infectious disease or an inflammatory disorder comprising administering to a subject in need thereof an effective amount of one or more cytokine-receptor activating agents and an effective amount of one or more co-stimulatory molecule-activating agents.

2. A method for treating cancer, said method comprising administering to a subject in need thereof an effective amount of a first compound that activates the IL-12 receptor, an effective amount of a second compound that activates 4-1BB, and an effective amount of a third compound that activates OX40.

3. A method for treating cancer, said method comprising administering to a subject in need thereof an effective amount of a first compound that activates the GM-CSF receptor and an effective amount of a first co-stimulatory molecule activating agent.

4. A method for treating cancer, said method comprising administering to a subject in need thereof an effective amount of a first compound that activates Flt3 and an effective amount of a first co-stimulatory molecule activating agent.

5. A method for treating cancer, said method comprising administering to a subject in need thereof an effective amount of a first compound that activates the GM-CSF receptor and an effective amount of a second compound that activates CD40.

6. The method of claim 2, wherein the first compound is IL-12 or a fragment, derivative or analog thereof, or an anti-IL-12 receptor antibody.

7. The method of claim 2, wherein the first compound is a nucleic acid molecule comprising the nucleotide sequence encoding IL-12 or a fragment, derivative or analog thereof, or an anti-IL-12 receptor antibody.

8. The method of claim 3, 4 or 5 which further comprises administering to said subject an effective amount of a second compound that activates the IL-12 receptor.

9. The method of claim 8, wherein the second compound is IL-12 or a fragment, derivative or analog thereof, or an anti-IL-12 receptor antibody.

10. The method of claim 8, wherein the second compound is a nucleic acid molecule comprising the nucleotide sequence encoding IL-12 or a fragment, derivative or analog thereof, or an anti-IL-12 receptor antibody.

11. The method of claim 3 or 5, wherein the first compound is GM-CSF or a fragment, derivative or analog thereof, or an anti-GM-CSF receptor antibody.

12. The method of claim 3 or 5, wherein the first compound is a nucleic acid molecule comprising the nucleotide sequence encoding GM-CSF or a fragment, derivative or analog thereof, or an anti-GM-CSF receptor antibody.

13. The method of claim 3 or 4, wherein the first co-stimulatory activating agent is a compound that activates 4-1BB, OX40, SLAM, ICOS, B7RP-1 or CD27.

14. The method of claim 3 or 4, wherein the first co-stimulatory activating agent is a compound that activates 4-1BB.

15. The method of claim 5 which further comprises administering to said subject a first co-stimulatory activating agent.

16. The method of claim 15, wherein the first co-stimulatory activating agent is a compound that activates 4-1BB, OX40, SLAM, ICOS, B7RP-1 or CD27.

17. The method of claim 2, wherein the second compound is 4-1BB ligand or a fragment, derivative or analog thereof, or an anti-4-1BB antibody.

18. The method of claim 2, wherein the second compound is a nucleic acid molecule comprising a nucleotide sequence encoding 4-1BB ligand or a fragment, derivative or analog thereof, or an anti-4-1BB antibody.

19. The method of claim 2, wherein the third compound is OX40 ligand or a fragment, derivative or analog thereof, or an anti-OX40 antibody.

20. The method of claim 2, wherein the third compound is a nucleic acid molecule comprising a nucleotide sequence encoding OX40 ligand or a fragment, derivative or analog thereof, or an anti-OX40 antibody.

21. The method of claim 7, wherein the expression of the nucleotide sequence encoding IL-12 or a fragment, derivative, or analog thereof, or an anti-IL-12 receptor antibody is regulated by a promoter.

22. The method of claim 10, wherein the expression of the nucleotide sequence encoding IL-12 or a fragment, derivative, or analog thereof, or an anti-IL-12 receptor antibody is regulated by a promoter.

23. The method of claim 12, wherein the expression of the nucleotide sequence encoding GM-CSF or a fragment, derivative, or analog thereof, or an anti-GM-CSF receptor antibody is regulated by a promoter.

24. The method of claim 18, wherein the expression of the nucleotide sequence encoding 4-1BB ligand or a fragment, derivative, or analog thereof, or an anti-4-1BB antibody is regulated by a promoter.

25. The method of claim 20, wherein the expression of the nucleotide sequence encoding OX40 ligand or a fragment, derivative, or analog thereof, or an anti-OX40 antibody is regulated by a promoter.

26. The method of claim 7, 12, 18 or 20, wherein the nucleic acid molecule is contained in an expression vector.

27. The method of claim 10, wherein the nucleic acid molecule is contained in an expression vector.

28. The method of claim 12, wherein the nucleic acid molecule is contained in an expression vector.

29. The method of claim 7, 12, 18 or 20, wherein the nucleic acid molecule is contained in a viral vector.

30. The method of claim 10, wherein the nucleic acid molecule is contained in a viral vector.

31. The method of claim 12, wherein the nucleic acid molecule is contained in a viral vector.

32. The method of claim 29, wherein the viral vector is an adenovirus vector, retroviral vector or an adeno-associated viral vector.

33. The method of claim 30, wherein the viral vector is an adenovirus vector, retroviral vector or an adeno-associated viral vector.

34. The method of claim 31, wherein the viral vector is an adenovirus vector, retroviral vector or an adeno-associated viral vector.

35. The method of claim 2, 3, 4 or 5, wherein the subject is a non-human mammal.

36. The method of claim 8, wherein the subject is a non-human mammal.

37. The method of claim 2, 3, 4 or 5, wherein the subject is a human.

38. The method of claim 8, wherein the subject is a human.

39. The method of claim 2, 3, 4 or 5, wherein the cancer is pancreatic cancer, breast cancer, ovarian cancer, prostate cancer, lung cancer or hepatic cancer.

40. The method of claim 8, wherein the cancer is pancreatic cancer, breast cancer, ovarian cancer, prostate cancer, lung cancer or hepatic cancer.
